(19) United States
(12) Patent Application Publication
(10) Pub. No.: US 2022/0117938 A1
(43) Pub. Date: Apr. 21, 2022

(54) COMBINATON THERAPY WITH A DON PRODRUG AND AN IMMUNE CHECKPOINT INHIBITOR

(71) Applicant: DRACEN PHARMACEUTICALS, INC., New York, NY (US)

(72) Inventors: ROBERT CHRISTIAN WILD, MURRIETA, CA (US); THOMAS ESTOK, LAKEWOOD RANCH, FL (US)

(21) Appl. No.: 17/423,819

(22) PCT Filed: Jan. 17, 2020
(86) PCT No.: PCT/US20/14149

§ 371 Date: Jul. 16, 2021

Related U.S. Application Data
(60) Provisional application No. 62/794,231, filed on Jan. 18, 2019.

(51) Int. Cl. A61K 31/4045; A61P 35/00; A61K 45/06; A61K 31/196; A61K 31/198; A61K 31/223; C07K 16/28

(52) U.S. Cl. CPC A61K 31/4045; A61P 35/00; A61K 45/06; C07K 16/2818; A61K 31/198; A61K 31/223; C07K 16/2827; A61K 31/196

(57) ABSTRACT

The present disclosure provides therapeutic methods of treating a cancer in a subject with isopropyl (S)-2-((S)-2-acetamido-3-(1H-indol-3-yl)propanamido)-6-diazo-5-oxohexanoate or isopropyl (S)-2-((S)-6-acetamido-2-((3S,5S,7S)-adamantane-1-carboxamido)hexanamido)-6-diazo-5-oxohexanoate, or (S)-2-((S)-2-acetamido-3-(1H-indol-3-yl)propanamido)-6-diazo-5-oxohexanoic acid, or DON and an immune checkpoint inhibitor. The present disclosure also provides intermittent dosing schedules for isopropyl (S)-2-((S)-2-acetamido-3-(1H-indol-3-yl)propanamido)-6-diazo-5-oxohexanoate or isopropyl (S)-2-((S)-6-acetamido-2-((3S,5S,7S)-adamantane-1-carboxamido)hexanamido)-6-diazo-5-oxohexanoate or (S)-2-((S)-2-acetamido-3-(1H-indol-3-yl)propanamido)-6-diazo-5-oxohexanoic acid for the treatment of cancer.

### BACKGROUND OF THE INVENTION

### Field of the Invention

The present disclosure provides therapeutic methods of treating a cancer in a subject with isopropyl (S)-2-((S)-2-acetamido-3-(1H-indol-3-yl)propanamido)-6-diazo-5-oxohexanoate, isopropyl (S)-2-((S)-6-acetamido-2-((3S,5S,7S)-adamantane-1-carboxamido)hexanamido)-6-diazo-5-oxohexanoate, (S)-2-((S)-2-acetamido-3-(1H-indol-3-yl)propanamido)-6-diazo-5-oxohexanoic acid, or 6-diazo-5-oxo-L-norleucine, and an immune checkpoint inhibitor. The present disclosure also provide intermittent dosing schedules for isopropyl (S)-2-((S)-2-acetamido-3-(1H-indol-3-yl)propanamido)-6-diazo-5-oxohexanoate, isopropyl (S)-2-((S)-6-acetamido-2-((3S,5S,7S)-adamantane-1-carboxamido)hexanamido)-6-diazo-5-oxohexanoate, or (S)-2-((S)-2-acetamido-3-(1H-indol-3-yl)propanamido)-6-diazo-5-oxohexanoic acid.

### Background

Dion et al., J. Am. Chem. Soc. 78:3075-3077 (1956) discloses 6-diazo-5-oxo-L-norleucine (DON) as a tumor-inhibitory substance. WO 2017/023774 discloses isopropyl (S)-2-((S)-2-acetamido-3-(1H-indol-3-yl)propanamido)-6-diazo-5-oxohexanoate and other prodrugs of DON. DON and DON prodrugs can be used to treat a variety of diseases, disorders, and conditions including, but not limited to, cancer, cognitive deficits, and metabolic reprogramming disorders. See WO 2017/023793, WO 2017/023791, WO 2017/023787, and PCT/US2018/54581.

### BRIEF SUMMARY OF THE INVENTION

In one aspect, the present disclosure provides therapeutic methods of treating a subject having cancer, the methods comprising administering to the subject a therapeutically effective amount of isopropyl (S)-2-((S)-2-acetamido-3-(1H-indol-3-yl)propanamido)-6-diazo-5-oxohexanoate, or a pharmaceutically acceptable salt thereof, (collectively referred to herein as “Compound 1”) or a therapeutically effective amount of isopropyl (S)-2-((S)-6-acetamido-2-((3S,5S,7S)-adamantane-1-carboxamido) hexanamido)-6-diazo-5-oxohexanoate, or a pharmaceutically acceptable salt thereof, (collectively referred to herein as “Compound 2”), or a therapeutically effective amount of (S)-2-((S)-2-acetamido-3-(1H-indol-3-yl)propanamido)-6-diazo-5-oxohexanoic acid, or a pharmaceutically acceptable salt thereof, (collectively referred to herein as “Compound 3”), or 6-diazo-5-oxo-L-norleucine, or a pharmaceutically acceptable salt thereof, (collectively referred to herein as “DON”), and a therapeutically effective amount of an immune checkpoint inhibitor, e.g., a PD-1 inhibitor, a PD-L1 inhibitor, a CTLA-4 inhibitor, a LAG3 inhibitor, a TIM3 inhibitor, or a cd47 inhibitor.

In another aspect, the present disclosure provides therapeutic methods of treating a subject having cancer, the methods comprising administering to the subject a therapeutically effective amount of Compound 1, Compound 2, or Compound 3 according to an intermittent dosing schedule.

In another aspect, the present disclosure provides therapeutic methods of treating a subject having cancer, the methods comprising administering to the subject a therapeutically effective amount DON for 5 consecutive days followed by 2 consecutive days when DON is not administered.

In one aspect, the present disclosure provides therapeutic methods of treating a subject having cancer, the methods comprising administering to the subject therapeutically effective amounts of Compound 1, Compound 2, Compound 3, or DON, and an immune checkpoint inhibitor according to an intermittent dosing schedule.

In another aspect, the present disclosure provides kits comprising Compound 1, Compound 2, Compound 3, or DON, and an immune checkpoint inhibitor.

### BRIEF DESCRIPTION OF DRAWINGS

FIG. 1 is a line graph showing the body weight change in female C57BL/6 mice bearing MC-38 tumors following administration of Compound 1. Data points represent group mean body weight. Error bars represent standard error of the mean (SEM).

FIG. 2 is a line graph showing the anti-tumor efficacy of Compound 1 in female C57BL/6 mice bearing MC-38 tumors.

FIG. 3 is a line graph showing the Kaplan-Meier survival curve of Compound 1 in female C57BL/6 mice bearing MC-38 tumors. **P<0.01, ***P<0.001. All groups compared to Group 1. Endpoint is defined as tumor volume reaching 2000 mm³.

FIG. 4 is a line graph showing the body weight change following administration of Compound 1 alone and in combination with anti-mPD-1 in female C57BL/6 mice bearing 4T1 tumors. Data points represent group mean body weight. Error bars represent standard error of the mean (SEM).

FIG. 5 is a line graph showing the anti-tumor efficacy of Compound 1 alone and in combination with anti-mPD-1 in female C57BL/6 mice bearing 4T1 tumors. *** P<0.001, All groups compared to Group 1 on Day 25; ^{###} P<0.001, All groups compared to Group 2 on Day 25; Two-Way RM ANOVA with Bonferroni posttests.

FIG. 6 is a line graph showing the Kaplan-Meier survival curve of Compound 1 alone and in combination with anti-mPD-1 in female C57BL/6 mice bearing 4T1 tumors. **P<0.01, ***P<0.001, All groups compared to Group 1; ^{#} P<0.01, ^{###} P<0.001, All groups compared to Group 2; Log-Rank test. Endpoint is defined as tumor volume reaching 2000 mm³.

FIG. 7 is a line graph showing the body weight change following administration of Compound 1 alone and in combination with anti-mPD-1 in female C57BL/6 mice bearing CT26.WT tumors. Data points represent group mean body weight. Error bars represent standard error of the mean (SEM).

FIG. 8 is a line graph showing the anti-tumor efficacy of Compound 1 alone and in combination with anti-mPD-1 in female C57BL/6 mice bearing CT26.WT tumors. *** P<0.001, All groups compared to Group 1; ^{###} P<0.001, All groups compared to Group 2.

FIG. 9 is a line graph showing the Kaplan-Meier survival curve of Compound 1 alone and in combination with anti-mPD-1 in female C57BL/6 mice bearing CT26.WT tumors. ***P<0.001, All groups compared to Group 1; ^{#} P<0.05, All groups compared to Group 2. Endpoint is defined as tumor volume reaching 2000 mm³.

FIG. 10 is a line graph showing the body weight change following administration of Compound 1 alone and in combination with anti-mPD-1 in female C57BL/6 mice bearing EL4 tumors. Data points represent group mean body weight. Error bars represent standard error of the mean (SEM).

FIG. 11 is a line graph showing the anti-tumor efficacy of Compound 1 alone and in combination with anti-mPD-1 in female C57BL/6 mice bearing EL4 tumors. ***P<0.001, All groups compared to Group 1.

FIG. 12 is a line graph showing the Kaplan-Meier survival curve of Compound 1 alone and in combination with anti-mPD-1 in female C57BL/6 mice bearing EL4 tumors. *P<0.05, ***P<0.001, All groups compared to Group 1. Endpoint is defined as tumor volume reaching 2000 mm³.

FIG. 13 is a line graph showing the body weight change following administration of Compound 1 alone and in combination with anti-mPD-1 in female C57BL/6 mice bearing MC-38 tumors. Data points represent group mean body weight. Error bars represent standard error of the mean (SEM).

FIG. 14 is a line graph showing the anti-tumor efficacy of Compound 1 alone and in combination with anti-mPD-1 in female C57BL/6 mice bearing MC-38 tumors. *** P<0.001, All groups compared to Group 1; ^{###} P<0.001, Group 4 and Group 6 compared to Group 2.

FIG. 15 is a line graph showing the Kaplan-Meier survival curve of Compound 1 alone and in combination with anti-mPD-1 in female C57BL/6 mice bearing MC-38 tumors. *** P<0.001, All groups compared to Group 1; ^{###} P<0.001, Group 4 and Group 6 compared to Group 2. Endpoint is defined as tumor volume reaching 2000 mm³.

FIG. 16 is a line graph showing the body weight change following administration of Compound 1 in female C57BL/6 mice bearing MC-38 tumors at different dosing schedules. Data points represent group mean body weight. Error bars represent standard error of the mean (SEM).

FIG. 17 is a line graph showing the anti-tumor efficacy of Compound 1 in female C57BL/6 mice bearing MC-38 tumors at different dosing schedules. *** P<0.001, All groups compared to Group 1.

FIG. 18 is a line graph showing the Kaplan-Meier survival curve of Compound 1 in female C57BL/6 mice bearing MC-38 tumors at different dosing schedules. *** P<0.001, All groups compared to Group 1. Endpoint is defined as tumor volume reaching 2000 mm³.

FIG. 19 is a line graph showing the body weight change following administration of Compound 1 alone and in combination with anti-mPD-1 in female C57BL/6 mice bearing MC-38 tumors. Data points represent group mean body weight. Error bars represent standard error of the mean (SEM).

FIG. 20 is a line graph showing the anti-tumor efficacy of Compound 1 alone and in combination with anti-mPD-1 in female C57BL/6 mice bearing MC-38 tumors. *** P<0.001, All groups compared to Group 1; ^{###} P<0.001, All groups compared to Group 2.

FIG. 21 is a line graph showing the Kaplan-Meier survival curve of Compound 1 alone and in combination with anti-mPD-1 in female C57BL/6 mice bearing MC-38 tumors. * P<0.05, *** P<0.001, All groups compared to Group 1; ^{###} P<0.001, All groups compared to Group 2. Endpoint is defined as tumor volume reaching 2000 mm³.

FIG. 22 is a line graph showing the anti-tumor efficacy of Compound 1 alone and in combination with anti-mPD-1 in female C57BL/6 mice bearing CT26.WT tumors.

FIG. 23 is a line graph showing the anti-tumor efficacy of Compound 1 alone and in combination with PD-L1 antibody in female C57BL/6 mice bearing H22 tumors.

FIG. 24 is a line graph showing the anti-tumor efficacy of Compound 1 in CT26.WT-bearing mice dosed subcutaneously at 1.4 mg/kg.

FIG. 25 is a line graph showing the Kaplan-Meier survival curve of Compound 1 in CT26.WT-bearing mice dosed subcutaneously at 1.4 mg/kg.

FIG. 26 is a line graph showing the anti-tumor efficacy of Compound 1 in CT26.WT-bearing mice dosed subcutaneously at equivalent total dose per cycle.

FIG. 27 is a line graph showing the Kaplan-Meier survival curve of Compound 1 in CT26.WT-bearing mice dosed subcutaneously at equivalent total dose per cycle.

FIG. 28 is a line graph showing the anti-tumor efficacy of Compound 1 in CT26.WT-bearing mice dosed subcutaneously at equivalent total dose per cycle comparing suboptimal doses.

FIG. 29 is a line graph showing the Kaplan-Meier survival curve of Compound 1 in CT26.WT-bearing mice dosed subcutaneously at equivalent total dose per cycle comparing suboptimal doses.

FIG. 30 is a line graph showing the anti-tumor efficacy of Compound 1 in CT26.WT tumor bearing mice dosed subcutaneously or intravenously at different dosing schedules. The definitions of Groups 1-6 are provided in EXAMPLE 8.

FIG. 31 is a line graph showing the anti-tumor efficacy of Compound 1 and Compound 3 in MC38 tumor bearing mice dosed subcutaneously or intravenously at different dosing schedules. The definitions of Groups 1-10 are provided in EXAMPLE 9.

FIG. 32 is a line graph showing the anti-tumor efficacy of Compound 1 and Compound 1 in combination with anti-PD-1 in CT26.WT tumor bearing mice dosed subcutaneously or intravenously at different dosing schedules. The definitions of Groups 1-10 are provided in EXAMPLE 10.

### DETAILED DESCRIPTION OF THE INVENTION

Applicant has unexpectedly discovered that intermittent dose administration of Compound 1, Compound 2, Compound 3, or DON maintains or improves the anti-cancer efficacy achieved with continuous dosing, but with less side-effects, e.g., less body weight loss, in preclinical animal models. In addition, intermittent dose administration of Compound 1, Compound 2, Compound 3, or DON induces an immune response in treated animals that unexpectedly inhibits tumor regrowth after tumor cell re-implantation. Applicant has also unexpectedly discovered that intermittent dose administration of Compound 1, Compound 2, Compound 3, or DON synergizes with immune checkpoint inhibitors (anti-PD-1 or anti-PD-L1) to produce durable tumor growth inhibition and significant increases in median survival time.

In one embodiment, the present disclosure provides therapeutic methods of treating a subject having cancer, the method comprising administering to the subject a therapeutically effective amount of Compound 1, Compound 2, Compound 3, or DON, and an immune checkpoint inhibitor, e.g., a PD-1 inhibitor, a PD-L1 inhibitor, a CTLA-4 inhibitor, a LAG3 inhibitor, a TIM3 inhibitor, or a cd47 inhibitor.

In one embodiment, the present disclosure provides therapeutic methods of treating a subject having cancer, the method comprising administering to the subject a therapeutically effective amount of Compound 1, Compound 2, Compound 3, or DON, and an immune checkpoint inhibitor, wherein Compound 1, Compound 2, Compound 3, or DON are administered to a subject according to an intermittent dosing schedule.

In another embodiment, Compound 1, Compound 2, Compound 3, or DON is administered to the subject before the immune checkpoint inhibitor.

In another embodiment, Compound 1, Compound 2, Compound 3, or DON is administered to the subject after the immune checkpoint inhibitor.

In another embodiment, Compound 1, Compound 2, Compound 3, or DON is administered to the subject at the same time as an immune checkpoint inhibitor.

In one embodiment, the present disclosure provides therapeutic methods of treating a subject having cancer, the method comprising administering to the subject a therapeutically effective amount of Compound 1, Compound 2, Compound 3, or DON (as single anti-cancer agent), wherein Compound 1, Compound 2, Compound 3, or DON is administered to a subject according to an intermittent dosing schedule.

In another embodiment, the present disclosure provides kits comprising Compound 1, Compound 2, Compound 3, or DON, and an immune checkpoint inhibitor, and instructions for administering Compound 1, Compound 2, Compound 3, or DON, and the immune checkpoint inhibitor to a subject having cancer.

In another embodiment, the kit is packaged in a manner that facilitates its use to practice methods of the present disclosure.

In another embodiment, the kit includes Compound 1, Compound 2, Compound 3, or DON (or a composition comprising Compound 1, Compound 2, Compound 3, or DON) packaged in a container, such as a sealed bottle or vessel, with a label affixed to the container or included in the kit that describes use of Compound 1, Compound 2, Compound 3, or DON or composition to practice the method of the disclosure. In one embodiment, Compound 1, Compound 2, Compound 3, or DON is packaged in a unit dosage form. The kit further can include a device suitable for administering the composition according to the intended route of administration.

The disclosure provides various therapeutic methods, kits, and compositions relating to the treatment of cancer. In one embodiment, the cancer is a solid tumor. In another embodiment, the cancer is a hematological cancer. In another embodiment, the cancer is any one or more of the cancers of Table 1.

TABLE 1

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic fibroma |
| lymphoma | rhabdomyosarcoma | sarcoma | |
| anaplastic large | anaplastic thyroid | angioimmunoblastic | angiomyolipoma |
| cell lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial tumor | |
| embryonal | endocrine gland | endodermal sinus | enteropathy- |
| carcinoma | neoplasm | tumor | associated T-cell |
| | | | lymphoma |
| esophageal cancer | fetus in fetu | fibroma | fibrosarcoma |
| follicular | follicular thyroid | ganglioneuroma | gastrointestinal |
| lymphoma | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck | hemangiopericytoma | hematological cancer | hepatoblastoma |
| cancer | | | |
| hepatosplenic T- | Hodgkin's lymphoma | non-Hodgkin's | invasive lobular |
| cell lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung cancer | non-small cell lung | MALT lymphoma |
| | | cancer | |
| malignant fibrous | malignant peripheral | malignant triton | mantle cell |
| histiocytoma | nerve sheath tumor | tumor | lymphoma |
| marginal zone B- | mast cell leukemia | mediastinal germ cell | medullary carcinoma |
| cell lymphoma | | tumor | of the breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid | myxoma | myxosarcoma | nasopharyngeal |
| liposarcoma | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell | renal medullary | retinoblastoma | rhabdomyoma |
| carcinoma | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal | squamous cell | synovial sarcoma | Sezary's disease |
| zone lymphoma | carcinoma | | |
| small intestine | squamous carcinoma | stomach cancer | T-cell lymphoma |
| cancer | | | |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

Exemplary hematological cancers include, but are not limited to, the cancers listed in Table 2. In another embodiment, the hematological cancer is acute lymphocytic leukemia, chronic lymphocytic leukemia (including B-cell chronic lymphocytic leukemia), or acute myeloid leukemia.

TABLE 2

| | |
|---|---|
| acute lymphocytic leukemia | acute eosinophilic leukemia |
| (ALL) | |
| acute myeloid leukemia (AML) | acute erythroid leukemia |
| chronic lymphocytic leukemia | acute lymphoblastic leukemia |
| (CLL) | |
| small lymphocytic lymphoma | acute megakaryoblastic leukemia |
| (SLL) | |
| multiple myeloma (MM) | acute monocytic leukemia |
| Hodgkins lymphoma (HL) | acute promyelocytic leukemia |
| non-Hodgkin's lymphoma | acute myelogeous leukemia |
| (NHL) | |
| mantle cell lymphoma (MCL) | B-cell prolymphocytic leukemia |
| marginal zone B-cell lymphoma | B-cell lymphoma |
| splenic marginal zone | MALT lymphoma |
| lymphoma | |
| follicular lymphoma (FL) | precursor T-lymphoblastic lymphoma |
| Waldenstrom's | T-cell lymphoma |
| macroglobulinemia (WM) | |
| diffuse large B-cell lymphoma | mast cell leukemia |
| (DLBCL) | |
| marginal zone lymphoma | adult T cell leukemia/lymphoma |
| (MZL) | |
| hairy cell leukemia (HCL) | aggressive NK-cell leukemia |
| Burkitt's lymphoma (BL) | angioimmunoblastic T-cell lymphoma |
| Richter's transformation | |

In another embodiment, the cancer is selected from the group consisting of squamous cell carcinoma of the head and neck, adenocarcinoma squamous cell carcinoma of the esophagus, adenocarcinoma of the stomach, adenocarcinoma of the colon, hepatocellular carcinoma, cholangiocarcinoma of the biliary system, adenocarcinoma of gall bladder, adenocarcinoma of the pancreas, ductal carcinoma in situ of the breast, adenocarcinoma of the breast, adenocarcinoma of the lungs, squamous cell carcinoma of the lungs, transitional cell carcinoma of the bladder, squamous cell carcinoma of the bladder, squamous cell carcinoma of the cervix, adenocarcinoma of the cervix, endometrial carcinoma, penile squamous cell carcinoma, and squamous cell carcinoma of the skin.

In another embodiment, a precancerous tumor is selected from the group consisting of leukoplakia of the head and neck, Barrett's esophagus, metaplasia of the stomach, adenoma of the colon, chronic hepatitis, bile duct hyperplasia, pancreatic intraepithelial neoplasia, atypical adenomatous hyperplasia of the lungs, dysplasia of the bladder, cervical initraepithelial neoplasia, penile intraepithelial neoplasia, and actinic keratosis of the skin.

In another embodiment, the cancer is selected from the group consisting of hepatocellular carcinoma, glioblastoma, lung cancer, breast cancer, head and neck cancer, prostate cancer, melanoma, and colorectal cancer.

In another embodiment, the cancer is selected from the group consisting of colorectal cancer, breast cancer, lymphoma, melanoma, kidney cancer, and lung cancer.

In another embodiment, the cancer has become resistant to conventional cancer treatments. The term “conventional cancer treatments” as used herein refers to any cancer drugs, biologics, or radiotherapy, or combination of cancer drugs and/or biologics and/or radiotherapy that have been tested and/or approved for therapeutic use in humans by the U.S. Food and Drug Administration, European Medicines Agency, or similar regulatory agency.

In another embodiment, the subject has been treated previously with an immune checkpoint inhibitor without Compound 1, Compound 2, Compound 3, or DON. For example, the previous immune checkpoint therapy may be an anti-PD-1 or anti-PD-L1 therapy.

In another embodiment, the present disclosure provides therapeutic methods of treating a subject having cancer, comprising administering to the subject therapeutically effective amounts of Compound 1, Compound 2, Compound 3, or DON, and an immune checkpoint inhibitor, wherein Compound 1, Compound 2, Compound 3, or DON is administered to the subject according to an intermittent dosing schedule.

In another embodiment, the present disclosure provides therapeutic methods of treating a subject having cancer, comprising administering to the subject a therapeutically effective amount of Compound 1, Compound 2, Compound 3, or DON, wherein Compound 1, Compound 2, Compound 3, or DON is administered to the subject according to an intermittent dosing schedule.

In another embodiment, the present disclosure provides therapeutic methods of treating a subject having cancer, comprising administering to the subject therapeutically effective amounts of Compound 1, Compound 2, Compound 3, or DON, an immune checkpoint inhibitor, and a third therapeutic agent.

In another embodiment, the present disclosure provides Compound 3, or a pharmaceutically acceptable salt thereof.

In another embodiment, the present disclosure provides a pharmaceutical composition comprising a Compound 3, or a pharmaceutically acceptable salt thereof, and a pharmaceutically acceptable excipient.

### I. DON and DON Prodrugs

6-Diazo-5-oxo-L-norleucine has the following structure:

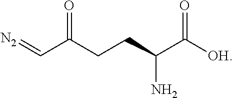

6-Diazo-5-oxo-L-norleucine, and the pharmaceutically acceptable salts thereof, are collectively referred to herein as “DON”. DON is disclosed in Dion et al., J. Am. Chem. Soc. 78:3075-3077 (1956).

Isopropyl (S)-2-((S)-2-acetamido-3-(1H-indol-3-yl)propanamido)-6-diazo-5-oxohexanoate has the following structure:

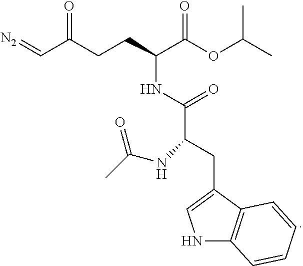

Isopropyl (S)-2-((S)-2-acetamido-3-(1H-indol-3-yl)propanamido)-6-diazo-5-oxohexanoate, and the pharmaceutically acceptable salts thereof, are collectively referred to herein as “Compound 1.” Compound 1 is disclosed in WO 2017/023774.

(S)-2-((S)-6-Acetamido-2-((3S,5S,7S)-adamantane-1-carboxamido)hexanamido)-6-diazo-5-oxohexanoate has the following structure:

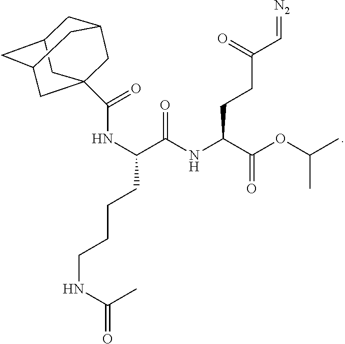

(S)-2-((S)-6-Acetamido-2-((3S,5S,7S)-adamantane-1-carboxamido)hexanamido)-6-diazo-5-oxohexanoate, and the pharmaceutically acceptable salt thereof, are collectively referred to herein as “Compound 2.” Compound 2 is disclosed in PCT/US2018/54581.

(S)-2-((S)-2-acetamido-3-(1H-indol-3-yl)propanamido)-6-diazo-5-oxohexanoic acid has the following structure:

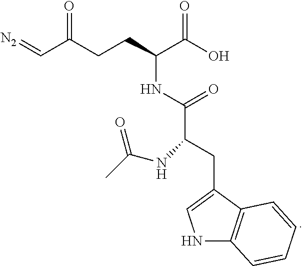

(S)-2-((S)-2-acetamido-3-(1H-indol-3-yl)propanamido)-6-diazo-5-oxohexanoic acid, and the pharmaceutically acceptable salts thereof, are collectively referred to herein as “Compound 3.”

Compound 1, Compound 2, Compound 3, or DON of the present disclosure may exist as pharmaceutically acceptable salts. Nonlimiting examples of salts of Compound 1, Compound 2, Compound 3, or DON include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerophosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts.

### II. Immune Checkpoint Inhibitors

Immune checkpoint inhibitors are therapies that blockade immune system inhibitor checkpoints. Immune checkpoints can be stimulatory or inhibitory. Blockade of inhibitory immune checkpoint activates immune system function and can be used for cancer immunotherapy. Pardoll, Nature Reviews. Cancer 12:252-64 (2012). Tumor cells turn off activated T cells when they attach to specific T-cell receptors. Immune checkpoint inhibitors prevent tumor cells from attaching to T cells, which results in T cells remaining activated. In effect, the coordinated action by cellular and soluble components combats pathogens and injuries by cancers. The modulation of immune system pathways may involve changing the expression or the functional activity of at least one component of the pathway to then modulate the response by the immune system. U.S. 2015/0250853. Examples of immune checkpoint inhibitors include PD-1 inhibitors, PD-L1 inhibitors, CTLA-4 inhibitors, LAG3 inhibitors, TIM3 inhibitors, cd47 inhibitors, and B7-H1 inhibitors. Thus, in one embodiment, the immune checkpoint inhibitor is selected from the group consisting of a PD-1 inhibitor, a PD-L1 inhibitor, a CTLA-4 inhibitor, a LAG3 inhibitor, a TIM3 inhibitor, and a cd47 inhibitor.

In another embodiment, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to nivolumab, pembrolizumab, STI-A1014, and pidilzumab. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In another embodiment, the immune checkpoint inhibitor is a PD-L1 (also known as B7-H1 or CD274) inhibitor. Examples of PD-L1 inhibitors include antibodies that specifically bind to PD-L1. Particular anti-PD-L1 antibodies include, but are not limited to, avelumab, atezolizumab, durvalumab, and BMS-936559. For a general discussion of the availability, methods of production, mechanism of action, and clinical studies, see U.S. Pat. No. 8,217,149, U.S. 2014/0341917, U.S. 2013/0071403, WO 2015036499, and Naido et al., British Journal of Cancer 111:2214-19 (2014).

In another embodiment, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In another embodiment, the immune checkpoint inhibitor is a LAG3 inhibitor. LAG3, Lymphocyte Activation Gene 3, is a negative co-simulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In another embodiment, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{C}1 T-cell responses. The TIM3 pathway is considered a target for anticancer immunotherapy due to its expression on dysfunctional CD8+ T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. Pat. No. 8,522,156, Ngiow et al., Cancer Res 71: 6567-71 (2011), Ngiow, et al., Cancer Res 71:3540-51 (2011), and Anderson, Cancer Immunology Res 2:393-98 (2014).

In another embodiment, the immune checkpoint inhibitor is a cd47 inhibitor. See Unanue, E. R., PNAS 110:10886-87 (2013).

The term “antibody” is meant to include intact monoclonal antibodies, polyclonal antibodies, multispecific antibodies formed from at least two intact antibodies, and antibody fragments, so long as they exhibit the desired biological activity. In another embodiment, “antibody” is meant to include soluble receptors that do not possess the Fc portion of the antibody. In one embodiment, the antibodies are humanized monoclonal antibodies and fragments thereof made by means of recombinant genetic engineering.

Another class of immune checkpoint inhibitors include polypeptides that bind to and block PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845.

Another class of immune checkpoint inhibitors include compounds with peptide moieties that inhibit PD-1 signaling. Examples of such compounds are disclosed in U.S. Pat. No. 8,907,053 and have the structure:

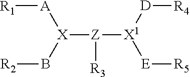

or a pharmaceutically acceptable salt thereof, wherein the compound comprises at least 5 amino acids useful as therapeutic agents capable of inhibiting the PD-1 signaling pathway.

Another class of immune checkpoint inhibitors include inhibitors of certain metabolic enzymes, such as indoleamine 2,3 dioxygenase (IDO), which is expressed by infiltrating myeloid cells and tumor cells. The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Löb, Cancer Immunol Immunother 58:153-57 (2009). Particular IDO blocking agents include, but are not limited to levo-1-methyl tryptophan (L-1MT) and 1-methyl-tryptophan (1MT). Qian et al., Cancer Res 69:5498-504 (2009); and Löb et al., Cancer Immunol Immunother 58:153-7 (2009).

In one embodiment, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736.

### III. Optional Therapeutic Agents

In certain therapeutic methods of the disclosure, a third therapeutic agent is administered to a subject having cancer in combination with Compound 1, Compound 2, Compound 3, or DON, and the immune checkpoint inhibitor. The third therapeutic agent used in the therapeutic methods of the present disclosure are referred to as “optional therapeutic agents.” Such optional therapeutic agents useful in the treatment of cancer patients are known in the art.

Optional therapeutic agents are administered in an amount to provide their desired therapeutic effect. The effective dosage range for each optional therapeutic agent is known in the art, and the optional therapeutic agent is administered to an individual in need thereof within such established ranges.

Compound 1, Compound 2, Compound 3, or DON, an immune checkpoint inhibitor, and/or the optional therapeutic agent can be administered together as a single-unit dose or separately as multi-unit doses, and in any order, e.g., wherein Compound 1, Compound 2, Compound 3, or DON is administered before the immune checkpoint inhibitor and/or the optional therapeutic agent, or vice versa. One or more doses of Compound 1, Compound 2, Compound 3, or DON, the immune checkpoint inhibitor, and/or the optional therapeutic agent can be administered to the subject.

In one embodiment, the optional therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat.

In another embodiment, the optional therapeutic agent is a chemotherapeutic agent or other anti-proliferative agent that can be administered in combination with Compound 1, Compound 2, Compound 3, or DON to treat cancer. Examples of conventional therapies and anticancer agents that can be used in combination with Compound 1, Compound 2, Compound 3, or DON include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved biologic therapy or chemotherapy, e.g., a treatment regimen that uses drugs to stop the growth of cancer cells, either by killing the cells or by stopping them from dividing. Chemotherapy may be given by mouth, injection, or infusion, or on the skin, depending on the type and stage of the cancer being treated.

Nonlimiting exemplary antiproliferative compounds include an aromatase inhibitor; an anti-estrogen; an anti-androgen; a gonadorelin agonist; a topoisomerase I inhibitor; a topoisomerase II inhibitor; a microtubule active agent; an alkylating agent, e.g., temozolomide; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Nonlimiting anti-estrogens include tamoxifen, fulvestrant, raloxifene, and raloxifene hydrochloride. Anti-androgens include, but are not limited to, bicalutamide. Gonadorelin agonists include, but are not limited to, abarelix, goserelin, and goserelin acetate.

Nonlimiting exemplary topoisomerase I inhibitors include topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; discodermolides; cochicine and epothilones and derivatives thereof.

Nonlimiting exemplary alkylating agents include cyclophosphamide, ifosfamide, melphalan, and nitrosoureas, such as carmustine and lomustine.

Nonlimiting exemplary matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Nonlimiting exemplary mTOR inhibitors include compounds that inhibit the mammalian target of rapamycin (mTOR) and possess antiproliferative activity such as sirolimus, everolimus, CCI-779, and ABT578.

Nonlimiting exemplary antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Nonlimiting exemplary platin compounds include carboplatin, cis-platin, cisplatinum, and oxaliplatin.

Nonlimiting exemplary methionine aminopeptidase inhibitors include bengamide or a derivative thereof and PPI-2458.

Nonlimiting exemplary bisphosphonates include etridonic acid, clodronic acid, tiludronic acid, pamidronic acid, alendronic acid, ibandronic acid, risedronic acid, and zoledronic acid.

Nonlimiting exemplary heparanase inhibitors include compounds that target, decrease, or inhibit heparin sulfate degradation, such as PI-88 and OGT2115.

Nonlimiting exemplary compounds which target, decrease, or inhibit the oncogenic activity of Ras include farnesyl transferase inhibitors, such as L-744832, DK8G557, tipifarnib, and lonafarnib.

Nonlimiting exemplary telomerase inhibitors include compounds that target, decrease, or inhibit the activity of telomerase, such as compounds that inhibit the telomerase receptor, such as telomestatin.

Nonlimiting exemplary proteasome inhibitors include compounds that target, decrease, or inhibit the activity of the proteasome including, but not limited to, bortezomib. In some embodiments, the proteasome inhibitor is carfilzomib.

Nonlimiting exemplary FMS-like tyrosine kinase inhibitors, which are compounds targeting, decreasing or inhibiting the activity of FMS-like tyrosine kinase receptors (Flt-3R) include interferon, I-β-D-arabinofuransylcytosine (ara-c), and bisulfan; and ALK inhibitors, which are compounds which target, decrease, or inhibit anaplastic lymphoma kinase.

Nonlimiting exemplary Flt-3 inhibitors include PKC412, midostaurin, a staurosporine derivative, SU11248, and MLN518.

Nonlimiting exemplary HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino,17-demethoxygeldanamycin (17AAG), a geldanamycin derivative; other geldanamycin related compounds; radicicol and HDAC inhibitors.

Nonlimiting exemplary protein tyrosine kinase and/or serine and/or threonine kinase inhibitors or lipid kinase inhibitors, include a) a compound targeting, decreasing, or inhibiting the activity of the platelet-derived growth factor-receptors (PDGFR), such as a compound that targets, decreases, or inhibits the activity of PDGFR, such as an N-phenyl-2-pyrimidine-amine derivatives, such as imatinib, SUlOl, SU6668, and GFB-111; b) a compound targeting, decreasing, or inhibiting the activity of the fibroblast growth factor-receptors (FGFR); c) a compound targeting, decreasing, or inhibiting the activity of the insulin-like growth factor receptor I (IGF-IR), such as a compound that targets, decreases, or inhibits the activity of IGF-IR; d) a compound targeting, decreasing, or inhibiting the activity of the Trk receptor tyrosine kinase family, or ephrin B4 inhibitors; e) a compound targeting, decreasing, or inhibiting the activity of the Axl receptor tyrosine kinase family; f) a compound targeting, decreasing, or inhibiting the activity of the Ret receptor tyrosine kinase; g) a compound targeting, decreasing, or inhibiting the activity of the Kit/SCFR receptor tyrosine kinase, such as imatinib; h) a compound targeting, decreasing, or inhibiting the activity of the c-Kit receptor tyrosine kinases, such as imatinib; i) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; j) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; k) a compound targeting, decreasing or inhibiting the activity of a protein-tyrosine kinase, such as imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) a compound targeting, decreasing, or inhibiting the activity of the epidermal growth factor family of receptor tyrosine kinases (EGFR, ErbB2, ErbB3, ErbB4 as homo- or heterodimers) and their mutants, such as CP 358774, ZD 1839, ZM 105180; trastuzumab, cetuximab, gefitinib, erlotinib, osimertinib, OSI-774, C1-1033, EKB-569, GW-2016, antibodies E1.1, E2.4, E2.5, E6.2, E6.4, E2.11, E6.3 and E7.6.3, and 7H-pyrrolo-[2,3-d]pyrimidine derivatives; and m) a compound targeting, decreasing, or inhibiting the activity of the c-Met receptor.

Nonlimiting exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, exemplary chemotherapeutic compounds, one or more of which may be used in combination with Compound 1, Compound 2, Compound 3, or DON include: avastin, daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, 1-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, bevacizumab, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

A number of suitable optional therapeutic, e.g., anticancer, agents are contemplated for use in the therapeutic methods provided herein. Indeed, the methods provided herein can include, but are not limited to, administration of numerous optional therapeutic agents such as: agents that induce apoptosis; polynucleotides (e.g., anti-sense, ribozymes, siRNA); polypeptides (e.g., enzymes and antibodies); biological mimetics (e.g., gossypol or BH3 mimetics); agents that bind (e.g., oligomerize or complex) with a Bcl-2 family protein such as Bax; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of optional therapeutic agents such as chemotherapeutic compounds and anticancer therapies suitable for co-administration with the disclosed compounds are known to those skilled in the art.

In certain embodiments, anticancer agents comprise agents that induce or stimulate apoptosis. Agents that induce or stimulate apoptosis include, for example, agents that interact with or modify DNA, such as by intercalating, cross-linking, alkylating, or otherwise damaging or chemically modifying DNA. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor. Additional anticancer agents include: vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the therapeutic methods provided herein include administering to a subject having cancer (a cancer patient) therapeutically effective amounts of Compound 1, Compound 2, Compound 3, or DON, and an immune checkpoint inhibitor and at least one additional anti-hyperproliferative or antineoplastic agent selected from alkylating agents, antimetabolites, and natural products (e.g., herbs and other plant and/or animal derived compounds).

Alkylating agents suitable for use in the present methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the methods of the present disclosure include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the therapeutic methods of the present disclosure. For example, the U.S. Food and Drug Administration (FDA) maintains a formulary of oncolytic agents approved for use in the United States. International counterpart agencies to the FDA maintain similar formularies. Those skilled in the art will appreciate that the “product labels” required on all U.S. approved chemotherapeutics describe approved indications, dosing information, toxicity data, and the like, for the exemplary agents.

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, vaproic acid, vinflunine, VNP40101M, volociximab, vorinostat, VX-680, ZD1839, ZD6474, zileuton, and zosuquidar trihydrochloride.

In one embodiment, the “optional therapeutic agent” comprises one of the anti-cancer drugs or anti-cancer drug combinations listed in Table A.

TABLE A

| | | Abraxane (Paclitaxel | |
|---|---|---|---|
| | | Albumin-stabilized | |
| | Abiraterone | Nanoparticle | |
| Abemaciclib | Acetate | Formulation) | ABVD |
| ABVE | ABVE-PC | AC | Acalabrutinib |
| AC-T | Actemra | Adcetris (Brentuximab | ADE |
| | (Tocilizumab) | Vedotin) | |
| Ado-Trastuzumab | Adriamycin | Afatinib Dimaleate | Afinitor |
| Emtansine | (Doxorubicin | | (Everolimus) |
| | Hydrochloride) | | |
| Akynzeo | Aldara | Aldesleukin | Alecensa |
| (Netupitant and | (Imiquimod) | | (Alectinib) |
| Palonosetron | | | |
| Hydrochloride) | | | |
| Alectinib | Alemtuzumab | Alimta (Pemetrexed | Aliqopa |
| | | Disodium) | (Copanlisib |
| | | | Hydrochloride) |
| Alkeran for | Alkeran Tablets | Aloxi (Palonosetron | Alunbrig |
| Injection | (Melphalan) | Hydrochloride) | (Brigatinib) |
| (Melphalan | | | |
| Hydrochloride) | | | |
| Ameluz | Amifostine | Aminolevulinic Acid | Anastrozole |
| (Aminolevulinic | | | |
| Acid) | | | |
| Apalutamide | Aprepitant | Aranesp (Darbepoetin | Aredia |
| | | Alfa) | (Pamidronate |
| | | | Disodium) |
| Arimidex | Aromasin | Arranon (Nelarabine) | Arsenic Trioxide |
| (Anastrozole) | (Exemestane) | | |
| Arzerra | Asparaginase | Atezolizumab | Avastin |
| (Ofatumumab) | Erwinia | | (Bevacizumab) |
| | chrysanthemi | | |
| Avelumab | Axicabtagene | Axitinib | Azacitidine |
| | Ciloleucel | | |
| Azedra | Bavencio | BEACOPP | Beleodaq |
| (Iobenguane 1131) | (Avelumab) | | (Belinostat) |
| Belinostat | Bendamustine | Bendeka (Bendamustine | BEP |
| | Hydrochloride | Hydrochloride) | |
| Besponsa | Bevacizumab | Bexarotene | Bicalutamide |
| (Inotuzumab | | | |
| Ozogamicin) | | | |
| BiCNU | Binimetinib | Bleomycin | Blinatumomab |
| (Carmustine) | | | |
| Blincyto | Bortezomib | Bosulif (Bosutinib) | Bosutinib |
| (Blinatumomab) | | | |
| Braftovi | Brentuximab | Brigatinib | BuMel |
| (Encorafenib) | Vedotin | | |
| Busulfan | Busulfex | Cabazitaxel | Cabometyx |
| | (Busulfan) | | (Cabozantinib-S- |
| | | | Malate) |
| Cabozantinib-S- | CAF | Calquence | Campath |
| Malate | | (Acalabrutinib) | (Alemtuzumab) |
| Camptosar | Capecitabine | CAPOX | Carac |
| (Irinotecan | | | (Fluorouracil- |
| Hydrochloride) | | | Topical) |
| Carboplatin | CARBOPLATIN- | Carfilzomib | Carmustine |
| | TAXOL | | |
| Carmustine | Casodex | CEM | Cemiplimab-rwlc |
| Implant | (Bicalutamide) | | |
| Ceritinib | Cerubidine | Cervarix (Recombinant | Cetuximab |
| | (Daunorubicin | HPV Bivalent Vaccine) | |
| | Hydrochloride) | | |
| CEV | Chlorambucil | CHLORAMBUCIL- | CHOP |
| | | PREDNISONE | |
| Cisplatin | Cladribine | Clofarabine | Clolar |
| | | | (Clofarabine) |
| CMF | Cobimetinib | Cometriq (Cabozantinib- | Copanlisib |
| | | S-Malate) | Hydrochloride |
| COPDAC | Copiktra | COPP | COPP-ABV |
| | (Duvelisib) | | |
| Cosmegen | Cotellic | Crizotinib | CVP |
| (Dactinomycin) | (Cobimetinib) | | |
| Cyclophosphamide | Cyramza | Cytarabine | Cytarabine |
| | (Ramucirumab) | | Liposome |
| Cytosar-U | Dabrafenib | Dacarbazine | Dacogen |
| (Cytarabine) | | | (Decitabine) |
| Dacomitinib | Dactinomycin | Daratumumab | Darbepoetin Alfa |
| Darzalex | Dasatinib | Daunorubicin | Daunorubicin |
| (Daratumumab) | | Hydrochloride | Hydrochloride |
| | | | and Cytarabine |
| | | | Liposome |
| Decitabine | Defibrotide | Defitelio (Defibrotide | Degarelix |
| | Sodium | Sodium) | |
| Denileukin | Denosumab | DepoCyt (Cytarabine | Dexamethasone |
| Diftitox | | Liposome) | |
| Dexrazoxane | Dinutuximab | Docetaxel | Doxil |
| Hydrochloride | | | (Doxorubicin |
| | | | Hydrochloride |
| | | | Liposome) |
| Doxorubicin | Doxorubicin | Dox-SL (Doxorubicin | Durvalumab |
| Hydrochloride | Hydrochloride | Hydrochloride | |
| | Liposome | Liposome) | |
| Duvelisib | Efudex | Eligard (Leuprolide | Elitek |
| | (Fluorouracil-- | Acetate) | (Rasburicase) |
| | Topical) | | |
| Ellence | Elotuzumab | Eloxatin (Oxaliplatin) | Eltrombopag |
| (Epirubicin | | | Olamine |
| Hydrochloride) | | | |
| Emend | Empliciti | Enasidenib Mesylate | Encorafenib |
| (Aprepitant) | (Elotuzumab) | | |
| Enzalutamide | Epirubicin | EPOCH | Epoetin Alfa |
| | Hydrochloride | | |
| Epogen (Epoetin | Erbitux | Eribulin Mesylate | Erivedge |
| Alfa) | (Cetuximab) | | (Vismodegib) |
| Erleada | Erlotinib | Erwinaze (Asparaginase | Ethyol |
| (Apalutamide) | Hydrochloride | Erwinia chrysanthemi) | (Amifostine) |
| Etopophos | Etoposide | Etoposide Phosphate | Evacet |
| (Etoposide | | | (Doxorubicin |
| Phosphate) | | | Hydrochloride |
| | | | Liposome) |
| Everolimus | Evista (Raloxifene | Evomela (Melphalan | Exemestane |
| | Hydrochloride) | Hydrochloride) | |
| 5-FU (Fluorouracil | 5-FU | Fareston (Toremifene) | Farydak |
| Injection) | (Fluorouracil-- | | (Panobinostat) |
| | Topical) | | |
| Faslodex | FEC | Femara (Letrozole) | Filgrastim |
| (Fulvestrant) | | | |
| Firmagon | Fludarabine | Fluoroplex (Fluorouracil-- | Fluorouracil |
| (Degarelix) | Phosphate | Topical) | Injection |
| Fluorouracil-- | Flutamide | FOLFIRI | FOLFIRI- |
| Topical | | | BEVACIZUMAB |
| FOLFIRI- | FOLFIRINOX | FOLFOX | Folotyn |
| CETUXIMAB | | | (Pralatrexate) |
| Fostamatinib | FU-LV | Fulvestrant | Fusilev |
| Disodium | | | (Leucovorin |
| | | | Calcium) |
| Gardasil | Gardasil 9 | Gazyva (Obinutuzumab) | Gefitinib |
| (Recombinant | (Recombinant | | |
| HPV Quadrivalent | HPV Nonavalent | | |
| Vaccine) | Vaccine) | | |
| Gemcitabine | GEMCITABINE- | GEMCITABINE- | Gemtuzumab |
| Hydrochloride | CISPLATIN | OXALIPLATIN | Ozogamicin |
| Gemzar | Gilotrif (Afatinib | Gleevec (Imatinib | Gliadel Wafer |
| (Gemcitabine | Dimaleate) | Mesylate) | (Carmustine |
| Hydrochloride) | | | Implant) |
| Glucarpidase | Goserelin Acetate | Granisetron | Granisetron |
| | | | Hydrochloride |
| Granix | Halaven (Eribulin | Hemangeol (Propranolol | Herceptin |
| (Filgrastim) | Mesylate) | Hydrochloride) | (Trastuzumab) |
| HPV Bivalent | HPV Nonavalent | HPV Quadrivalent | Hycamtin |
| Vaccine, | Vaccine, | Vaccine, Recombinant | (Topotecan |
| Recombinant | Recombinant | | Hydrochloride) |
| Hydrea | Hydroxyurea | Hyper-CVAD | Ibrance |
| (Hydroxyurea) | | | (Palbociclib) |
| Ibritumomab | Ibrutinib | ICE | Iclusig (Ponatinib |
| Tiuxetan | | | Hydrochloride) |
| Idarubicin | Idelalisib | Idhifa (Enasidenib | Ifex (Ifosfamide) |
| Hydrochloride | | Mesylate) | |
| Ifosfamide | IL-2 | Imatinib Mesylate | Imbruvica |
| | (Aldesleukin) | | (Ibrutinib) |
| Imfinzi | Imiquimod | Imlygic (Talimogene | Inlyta (Axitinib) |
| (Durvalumab) | | Laherparepvec) | |
| Inotuzumab | Interferon Alfa- | Interleukin-2 | Intron A |
| Ozogamicin | 2b, Recombinant | (Aldesleukin) | (Recombinant |
| | | | Interferon Alfa- |
| | | | 2b) |
| Iobenguane 1131 | Ipilimumab | Iressa (Gefitinib) | Irinotecan |
| | | | Hydrochloride |
| Irinotecan | Istodax | Ivosidenib | Ixabepilone |
| Hydrochloride | (Romidepsin) | | |
| Liposome | | | |
| Ixazomib Citrate | Ixempra | Jakafi (Ruxolitinib | JEB |
| | (Ixabepilone) | Phosphate) | |
| Jevtana | Kadcyla (Ado- | Kepivance (Palifermin) | Keytruda |
| (Cabazitaxel) | Trastuzumab | | (Pembrolizumab) |
| | Emtansine) | | |
| Kisqali | Kymriah | Kyprolis (Carfilzomib) | Lanreotide |
| (Ribociclib) | (Tisagenlecleucel) | | Acetate |
| Lapatinib | Larotrectinib | Lartruvo (Olaratumab) | Lenalidomide |
| Ditosylate | Sulfate | | |
| Lenvatinib | Lenvima | Letrozole | Leucovorin |
| Mesylate | (Lenvatinib | | Calcium |
| | Mesylate) | | |
| Leukeran | Leuprolide | Levulan | Libtayo |
| (Chlorambucil) | Acetate | Kerastik (Aminolevulinic | (Cemiplimab- |
| | | Acid) | rwlc) |
| LipoDox | Lomustine | Lonsurf (Trifluridine and | Lorbrena |
| (Doxorubicin | | Tipiracil Hydrochloride) | (Lorlatinib) |
| Hydrochloride | | | |
| Liposome) | | | |
| Lorlatinib | Lumoxiti | Lupron (Leuprolide | Lupron Depot |
| | (Moxetumomab | Acetate) | (Leuprolide |
| | Pasudotox-tdfk) | | Acetate) |
| Lutathera | Lutetium (Lu 177- | Lynparza (Olaparib) | Marqibo |
| (Lutetium Lu 177- | Dotatate) | | (Vincristine |
| Dotatate) | | | Sulfate |
| | | | Liposome) |
| Matulane | Mechlorethamine | Megestrol Acetate | Mekinist |
| (Procarbazine | Hydrochloride | | (Trametinib) |
| Hydrochloride) | | | |
| Mektovi | Melphalan | Melphalan | Mercaptopurine |
| (Binimetinib) | | Hydrochloride | |
| Mesna | Mesnex (Mesna) | Methotrexate | Methylnaltrexone |
| | | | Bromide |
| Midostaurin | Mitomycin C | Mitoxantrone | Mogamulizumab- |
| | | Hydrochloride | kpke |
| Moxetumomab | Mozobil | Mustargen | MVAC |
| Pasudotox-tdfk | (Plerixafor) | (Mechlorethamine | |
| | | Hydrochloride) | |
| Myleran | Mylotarg | Nanoparticle Paclitaxel | Navelbine |
| (Busulfan) | (Gemtuzumab | (Paclitaxel Albumin- | (Vinorelbine |
| | Ozogamicin) | stabilized Nanoparticle | Tartrate) |
| | | Formulation) | |
| Necitumumab | Nelarabine | Neratinib Maleate | Nerlynx |
| | | | (Neratinib |
| | | | Maleate) |
| Netupitant and | Neulasta | Neupogen (Filgrastim) | Nexavar |
| Palonosetron | (Pegfilgrastim) | | (Sorafenib |
| Hydrochloride | | | Tosylate) |
| Nilandron | Nilotinib | Nilutamide | Ninlaro |
| (Nilutamide) | | | (Ixazomib |
| | | | Citrate) |
| Niraparib Tosylate | Nivolumab | Nplate (Romiplostim) | Obinutuzumab |
| Monohydrate | | | |
| Odomzo | OEPA | Ofatumumab | OFF |
| (Sonidegib) | | | |
| Olaparib | Olaratumab | Omacetaxine | Oncaspar |
| | | Mepesuccinate | (Pegaspargase) |
| Ondansetron | Onivyde | Ontak (Denileukin | Opdivo |
| Hydrochloride | (Irinotecan | Diftitox) | (Nivolumab) |
| | Hydrochloride | | |
| | Liposome) | | |
| OPPA | Osimertinib | Oxaliplatin | Paclitaxel |
| Paclitaxel | PAD | Palbociclib | Palifermin |
| Albumin-stabilized | | | |
| Nanoparticle | | | |
| Formulation | | | |
| Palonosetron | Palonosetron | Pamidronate Disodium | Panitumumab |
| Hydrochloride | Hydrochloride | | |
| | and Netupitant | | |
| Panobinostat | Pazopanib | PCV | PEB |
| | Hydrochloride | | |
| Pegaspargase | Pegfilgrastim | Peginterferon Alfa-2b | PEG-Intron |
| | | | (Peginterferon |
| | | | Alfa-2b) |
| Pembrolizumab | Pemetrexed | Perjeta (Pertuzumab) | Pertuzumab |
| | Disodium | | |
| Plerixafor | Pomalidomide | Pomalyst | Ponatinib |
| | | (Pomalidomide) | Hydrochloride |
| Portrazza | Poteligeo | Pralatrexate | Prednisone |
| (Necitumumab) | (Mogamulizumab- | | |
| | kpkc) | | |
| Procarbazine | Procrit (Epoetin | Proleukin (Aldesleukin) | Prolia |
| Hydrochloride | Alfa) | | (Denosumab) |
| Promacta | Propranolol | Provenge (Sipuleucel-T) | Purinethol |
| (Eltrombopag | Hydrochloride | | (Mercaptopurine) |
| Olamine) | | | |
| Purixan | Radium 223 | Raloxifene | Ramucirumab |
| (Mercaptopurine) | Dichloride | Hydrochloride | |
| Rasburicase | R-CHOP | R-CVP | Recombinant |
| | | | Human |
| | | | Papillomavirus |
| | | | (HPV) Bivalent |
| | | | Vaccine |
| Recombinant | Recombinant | Recombinant Interferon | Regorafenib |
| Human | Human | Alfa-2b | |
| Papillomavirus | Papillomavirus | | |
| (HPV) Nonavalent | (HPV) | | |
| Vaccine | Quadrivalent | | |
| | Vaccine | | |
| Relistor | R-EPOCH | Retacrit (Epoetin Alfa) | Revlimid |
| (Methylnaltrexone | | | (Lenalidomide) |
| Bromide) | | | |
| Rheumatrex | Ribociclib | R-ICE | Rituxan |
| (Methotrexate) | | | (Rituximab) |
| Rituxan Hycela | Rituximab | Rituximab and | Rolapitant |
| (Rituximab and | | Hyaluronidase Human | Hydrochloride |
| Hyaluronidase | | | |
| Human) | | | |
| Romidepsin | Romiplostim | Rubidomycin | Rubraca |
| | | (Daunorubicin | (Rucaparib |
| | | Hydrochloride) | Camsylate) |
| Rucaparib | Ruxolitinib | Rydapt (Midostaurin) | Sancuso |
| Camsylate | Phosphate | | (Granisetron) |
| Sclerosol | Siltuximab | Sipuleucel-T | Somatuline Depot |
| Intrapleural | | | (Lanreotide |
| Aerosol (Talc) | | | Acetate) |
| Sonidegib | Sorafenib | Sprycel (Dasatinib) | STANFORD V |
| | Tosylate | | |
| Sterile Talc | Steritalc (Talc) | Stivarga (Regorafenib) | Sunitinib Malate |
| Powder (Talc) | | | |
| Sustol | Sutent (Sunitinib | Sylatron (Peginterferon | Sylvant |
| (Granisetron) | Malate) | Alfa-2b) | (Siltuximab) |
| Synribo | Tabloid | TAC | Tafinlar |
| (Omacetaxine | (Thioguanine) | | (Dabrafenib) |
| Mepesuccinate) | | | |
| Tagrisso | Talc | Talimogene | Tamoxifen |
| (Osimertinib) | | Laherparepvec | Citrate |
| Tarabine PFS | Tarceva (Erlotinib | Targretin (Bexarotene) | Tasigna |
| (Cytarabine) | Hydrochloride) | | (Nilotinib) |
| Tavalisse | Taxol (Paclitaxel) | Taxotere (Docetaxel) | Tecentriq |
| (Fostamatinib | | | (Atezolizumab) |
| Disodium) | | | |
| Temodar | Temozolomide | Temsirolimus | Thalidomide |
| (Temozolomide) | | | |
| Thalomid | Thioguanine | Thiotepa | Tibsovo |
| (Thalidomide) | | | (Ivosidenib) |
| Tisagenlecleucel | Tocilizumab | Tolak (Fluorouracil-- | Topotecan |
| | | Topical) | Hydrochloride |
| Toremifene | Torisel | Totect (Dexrazoxane | TPF |
| | (Temsirolimus) | Hydrochloride) | |
| Trabectedin | Trametinib | Trastuzumab | Treanda |
| | | | (Bendamustine |
| | | | Hydrochloride) |
| Trexall | Trifluridine and | Trisenox (Arsenic | Tykerb (Lapatinib |
| (Methotrexate) | Tipiracil | Trioxide) | Ditosylate) |
| | Hydrochloride | | |
| Unituxin | Uridine Triacetate | VAC | Valrubicin |
| (Dinutuximab) | | | |
| Valstar | Vandetanib | VAMP | Varubi |
| (Valrubicin) | | | (Rolapitant |
| | | | Hydrochloride) |
| Vectibix | VeIP | Velcade (Bortezomib) | Vemurafenib |
| (Panitumumab) | | | |
| Venclexta | Venetoclax | Verzenio (Abemaciclib) | Vidaza |
| (Venetoclax) | | | (Azacitidine) |
| Vinblastine Sulfate | Vincristine | Vincristine Sulfate | Vinorelbine |
| | Sulfate | Liposome | Tartrate |
| VIP | Vismodegib | Vistogard (Uridine | Vitrakvi |
| | | Triacetate) | (Larotrectinib |
| | | | Sulfate) |
| Vizimpro | Voraxaze | Vorinostat | Votrient |
| (Dacomitinib) | (Glucarpidase) | | (Pazopanib |
| | | | Hydrochloride) |
| Vyxeos | Xalkori | Xeloda (Capecitabine) | XELIRI |
| (Daunorubicin | (Crizotinib) | | |
| Hydrochloride and | | | |
| Cytarabine | | | |
| Liposome) | | | |
| XELOX | Xgeva | Xofigo (Radium 223 | Xtandi |
| | (Denosumab) | Dichloride) | (Enzalutamide) |
| Yervoy | Yescarta | Yondelis (Trabectedin) | Zaltrap (Ziv- |
| (Ipilimumab) | (Axicabtagene | | Aflibercept) |
| | Ciloleucel) | | |
| Zarxio (Filgrastim) | Zejula (Niraparib | Zelboraf (Vemurafenib) | Zevalin |
| | Tosylate | | (Ibritumomab |
| | Monohydrate) | | Tiuxetan) |
| Zinecard | Ziv-Aflibercept | Zofiran (Ondansetron | Zoladex |
| (Dexrazoxane | | Hydrochloride) | (Goserelin |
| Hydrochloride) | | | Acetate) |
| Zoledronic Acid | Zolinza | Zometa (Zoledronic | Zydelig |
| | (Vorinostat) | Acid) | (Idelalisib) |
| Zykadia | Zytiga | | |
| (Ceritinib) | (Abiraterone | | |
| | Acetate) | | |

For a more detailed description of anticancer agents and other optional therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

In some embodiments, methods provided herein comprise administering Compound 1, Compound 2, Compound 3, or DON, and an immune checkpoint inhibitor to a cancer patient in combination with radiation therapy. The methods provided herein are not limited by the types, amounts, or delivery and administration systems used to deliver the therapeutic dose of radiation to a patient. For example, the patient may receive photon radiotherapy, particle beam radiation therapy, other types of radiotherapies, and combinations thereof. In some embodiments, the radiation is delivered to the patient using a linear accelerator. In still other embodiments, the radiation is delivered using a gamma knife.

The source of radiation can be external or internal to the patient. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by patients. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The patient may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1. IL-6, and the like). Radiosensitizers enhance the killing of tumor cells. Radioprotectors protect healthy tissue from the harmful effects of radiation.

Any type of radiation can be administered to a patient, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to a patient is about 0.01 Gray (Gy) to about 100 Gy. In another embodiment, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the methods provided herein.

### IV. Therapeutic Methods

In the therapeutic methods provided herein, Compound 1, Compound 2, Compound 3, or DON can by administered to a subject having cancer as a single chemotherapeutic agent. Compound 1, Compound 2, Compound 3, or DON can also be administered to a subject having cancer in combination with an immune checkpoint inhibitor. Compound 1, Compound 2, Compound 3, or DON, and the immune checkpoint inhibitor can be administered in combination under one or more of the following conditions: at different periodicities, at different durations, at different concentrations, by different administration routes, etc. An optional therapeutic, e.g., anticancer, agent may also be administered to the cancer patient. In some embodiments, Compound 1, Compound 2, Compound 3, or DON are administered to the patient according to an intermittent dosing schedule. In some embodiments, Compound 1, Compound 2, Compound 3, or DON are subcutaneously administered to the patient according to an intermittent dosing schedule. In some embodiments, Compound 1, Compound 2, Compound 3, or DON are intravenously administered to the patient according to an intermittent dosing schedule

In some embodiments, Compound 1, Compound 2, Compound 3, or DON is administered prior to the immune checkpoint inhibitor, e.g., 0.5, 1, 2, 3, 4, 5, 10, 12, or 18 hours, 1, 2, 3, 4, 5, or 6 days, or 1, 2, 3, or 4 weeks prior to the administration of the immune checkpoint inhibitor.

In some embodiments, Compound 1, Compound 2, Compound 3, or DON is administered after the immune checkpoint inhibitor, e.g., 0.5, 1, 2, 3, 4, 5, 10, 12, or 18 hours, 1, 2, 3, 4, 5, or 6 days, or 1, 2, 3, or 4 weeks after the administration of the immune checkpoint inhibitor.

In some embodiments, Compound 1, Compound 2, Compound 3, or DON, and the immune checkpoint inhibitor are administered concurrently but on different schedules, e.g., Compound 1, Compound 2, Compound 3, or DON is administered daily while the immune checkpoint inhibitor is administered once a week, once every two weeks, once every three weeks, or once every four weeks. In other embodiments, Compound 1, Compound 2, Compound 3, or DON is administered once a day while the immune checkpoint inhibitor is administered once a week, once every two weeks, once every three weeks, or once every four weeks.

The therapeutic methods provided herein comprise administering Compound 1, Compound 2, Compound 3, or DON to a cancer patient in an amount which is effective to achieve its intended purpose. While individual needs vary, determination of optimal ranges of effective amounts of each component is within the skill of the art. Typically, Compound 1, Compound 2, Compound 3, or DON is administered in an amount from about 0.05 mg/kg to about 500 mg/kg, about 0.05 mg/kg to about 100 mg/kg, about 0.05 mg/kg to about 50 mg/kg, or about 0.05 mg/kg to about 10 mg/kg. The dosage of a composition can be at any dosage including, but not limited to, about 0.05 mg/week to about 25 mg/week. Particular doses include 0.05, 1, 2, 5, 10, 20, 500, and 100 mg/kg once weekly. In one embodiment, Compound 1, Compound 2, Compound 3, or DON is administered once a week. These dosages are exemplary, but there can be individual instances in which higher or lower dosages are merited, and such are within the scope of this disclosure. In practice, the physician determines the actual dosing regimen that is most suitable for an individual patient, which can vary with the age, weight, and response of the particular patient. In one embodiment, about 0.1 mg/kg to about 2 mg/kg of DON is administered to the subject.

The unit oral dose of Compound 1, Compound 2, Compound 3, or DON may comprise from about 0.01 to about 1000 mg, e.g., about 0.01 to about 100 mg of Compound 1, Compound 2, Compound 3, or DON. In one embodiment, the unit oral dose of Compound 1, Compound 2, Compound 3, or DON is 0.05 mg, 1 mg, 3 mg, 5 mg, 7 mg, 9 mg, 10 mg 12 mg, 14 mg, 15 mg, 17 mg, 20 mg, 22 mg, 25 mg, 27 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg. The unit dose may be administered one or more times daily, e.g., as one or more tablets or capsules. The unit does may also be administered by IV or subcutaneously to the subject. In practice, the physician determines the actual dosing regimen that is most suitable for an individual patient, which can vary with the age, weight, and response of the particular patient.

In addition to administering Compound 1, Compound 2, Compound 3, or DON as a raw chemical, it may be administered as part of a pharmaceutical preparation or composition. In some embodiments, the pharmaceutical preparation or composition can include one or more pharmaceutically acceptable carriers, excipients, and/or auxiliaries. In some embodiments, the one or more carriers, excipients, and auxiliaries facilitate processing of Compound 1, Compound 2, Compound 3, or DON into a preparation or composition which can be used pharmaceutically. The preparations, particularly those preparations which can be administered orally, subcutaneously, intravenously, or topically, and which can be used for one type of administration, such as tablets, dragees, slow release lozenges and capsules, mouth rinses and mouth washes, gels, liquid suspensions, hair rinses, hair gels, and shampoos, and also preparations which can be administered rectally, such as suppositories, as well as suitable solutions for administration by intravenous infusion, subcutaneous injection, topically or orally, contain from about 0.01 to 99 percent, in one embodiment from about 0.25 to 75 percent of active compound(s), together with the one or more carriers, excipients, and/or auxiliaries.

The pharmaceutical compositions of provided herein may be administered to any subject which may experience the beneficial effects of Compound 1, Compound 2, Compound 3, or DON. Foremost among such subjects are mammals, e.g., humans, although the methods and compositions provided herein are not intended to be so limited. Other subjects include veterinary animals (cows, sheep, pigs, horses, dogs, cats and the like). In one embodiment, the subject is a human cancer patient.

The pharmaceutical preparations provided herein are manufactured by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries can be suitable flow-regulating agents and lubricants. Suitable auxiliaries include, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension including, for example, sodium carboxymethyl cellulose, sorbitol, and/or dextran. Optionally, the suspension may also contain stabilizers.

The present disclosure encompasses the use of solvates of Compound 1, Compound 2, Compound 3, or DON. Solvates typically do not significantly alter the physiological activity or toxicity of a compound, and as such may function as pharmacological equivalents. The term “solvate” as used herein is a combination, physical association and/or solvation of Compound 1, Compound 2, Compound 3, or DON with a solvent molecule such as, e.g., a disolvate, monosolvate or hemisolvate, where the ratio of solvent molecule to Compound 1, Compound 2, Compound 3, or DON is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compound 1, Compound 2, Compound 3, or DON can be present as solvated forms with a pharmaceutically acceptable solvent, such as water, methanol, ethanol, and the like, and it is intended that the disclosure includes both solvated and unsolvated forms of Compound 1, Compound 2, Compound 3, or DON. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1):Article 12 (2004), and A. L. Bingham et al., Chem. Commun. 603-604 (2001). A typical, non-limiting, process of preparing a solvate involves dissolving Compound 1, Compound 2, Compound 3, or DON in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

Therapeutically effective amounts of Compound 1, Compound 2, Compound 3, or DON, and the immune checkpoint inhibitor formulated in accordance with standard pharmaceutical practices, are administered to a human patient in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

Compound 1, Compound 2, Compound 3, or DON, and the immune checkpoint inhibitor can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique. In one embodiment, Compound 1, Compound 2, Compound 3, or DON is administered subcutaneously to the subject. In one embodiment, Compound 1, Compound 2, Compound 3, or DON is administered intravenously to the subject.

Pharmaceutical compositions include those wherein Compound 1, Compound 2, Compound 3, or DON, and the immune checkpoint inhibitor are administered in an effective amount to achieve its intended purpose. The exact formulation, route of administration, and dosage is determined by an individual physician in view of the diagnosed condition or disease. Dosage amount and interval can be adjusted individually to provide levels of Compound 1, Compound 2, Compound 3, or DON, and the immune checkpoint inhibitor that is sufficient to maintain therapeutic effects.

Toxicity and therapeutic efficacy of Compound 1, Compound 2, Compound 3, or DON, and the immune checkpoint inhibitor can be determined by standard pharmaceutical procedures in cell cultures or experimental animals, e.g., for determining the maximum tolerated dose (MTD) of a compound, which defines as the highest dose that causes no toxicity in a patient. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of Compound 1, Compound 2, Compound 3, or DON, and the immune checkpoint inhibitor required for use in therapy varies with the nature of the condition being treated, the length of time that activity is desired, and the age and the condition of the subject, and ultimately is determined by the attendant physician. For example, dosage amounts and intervals can be adjusted individually to provide plasma levels of Compound 1, Compound 2, Compound 3, or DON, and immune checkpoint inhibitor that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, Compound 1, Compound 2, Compound 3, or DON, and immune checkpoint inhibitor can be administered at a frequency of: one dose per day; four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

The immune checkpoint inhibitor is administered in therapeutically effective amounts. When the immune checkpoint inhibitor is a monoclonal antibody, 1-20 mg/kg is administered as an intravenous infusion every 2-4 weeks. For example, 50 mg, 60 mg, 70 mg, 80 mg, 90 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg and 2000 mg of the antibody may be administered.

For example, when the immune checkpoint inhibitor is the anti-PD-1 antibody nivolumab, 3 mg/kg may be administered by intravenous infusion over 60 minutes every two weeks. When the immune checkpoint inhibitor is the anti-PD-1 antibody pembrolizumab, mg/kg may be administered by intravenous infusion over 30 minutes every two or three weeks. When the immune checkpoint inhibitor is the anti-PD-L1 antibody avelumab, 10 mg/kg may be administered by intravenous infusion as frequently as every 2 weeks. Disis et al., J. Clin Oncol. 33 (2015) (suppl; abstr 5509). When the immune checkpoint inhibitor is the anti-PD-L1 antibody MPDL3280A, 20 mg/kg may be administered by intravenous infusion every 3 weeks. Herbst et al., Nature 515:563-80 (2014). When the immune checkpoint inhibitor is the anti-CTLA-4 antibody ipilumumab, 3 mg/kg may be administered by intravenous infusion over 90 minutes every 3 weeks. When the immune checkpoint inhibitor is the anti-CTLA-4 antibody tremelimumab, 15 mg/kg may be administered by intravenous infusion every 12 weeks. Naido et al., British Journal of Cancer 111:2214-19 (2014); Drugs R D, 10:123-32 (2010). When the immune checkpoint inhibitor is the anti-LAG3 antibody GSK2831781, 1.5 to 5 mg/kg may be administered by intravenous infusion over 120 minutes every 2-4 weeks. When the immune checkpoint inhibitor is an anti-TIM3 antibody, 1-5 mg/kg may be administered by intravenous infusion over 30-90 minutes every 2-4 weeks. When an inhibitor of indoleamine 2,3-dioxygenase (IDO) pathway is inhibitor indoximod in combination with temozolomide, 18.5 mg/kg/dose BID with an escalation to 27.7 mg/kg/dose BID of indoximod with 200 mg/m² every 5 days of temozolomide.

In one embodiment, the immune checkpoint inhibitor is an antibody and 1-20 mg/kg is administered by intravenous infusion every 2-4 weeks. In another embodiment, 50-2000 mg of the antibody is administered by intravenous infusion every 2-4 weeks. In another embodiment, Compound 1, Compound 2, Compound 3, or DON is administered prior to administration of the antibody. In another embodiment, Compound 1, Compound 2, Compound 3, or DON is administered 3-7 days prior to the day of administration of the antibody. In another embodiment, Compound 1, Compound 2, Compound 3, or DON is also administered the day the antibody is administered and on consecutive days thereafter until disease progression or until Compound 1, Compound 2, Compound 3, or DON administration is no longer beneficial.

In one embodiment, the cancer patient receives 2 mg/kg pembrolizumab administered by intravenous infusion every three weeks and about 0.1 to 100 mg of Compound 1, Compound 2, Compound 3, or DON administered for 1-7 days prior to pembrolizumab administration, optionally, on the day of pembrolizumab administration, and, optionally, thereafter until disease progression or until there is no therapeutic benefit.

In another embodiment, the cancer patient receives 3 mg/kg nivolumab administered by intravenous infusion every 2 weeks and about 0.1 to 100 mg of Compound 1, Compound 2, Compound 3, or DON administered for 1-7 days prior to nivolumab administration, optionally, on the day of nivolumab administration, and, optionally, thereafter until disease progression or until there is no therapeutic benefit.

In another embodiment, the cancer patient receives 3 mg/kg nivolumab administered by intravenous infusion every 2 weeks and about 0.1 to 100 mg of Compound 1, Compound 2, Compound 3, or DON administered for 1-7 days prior to nivolumab administration, optionally, on the day of nivolumab administration, and, optionally, thereafter until disease progression or until there is no therapeutic benefit.

In another embodiment, the treatment of the cancer patient with Compound 1, Compound 2, Compound 3, or DON, and an immune checkpoint inhibitor induces anti-proliferative response faster than when the immune checkpoint inhibitor is administered alone.

### V. Definitions

The terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language, e.g., “such as,” provided herein, is intended to better illustrate the disclosure and is not a limitation on the scope of the disclosure unless otherwise claimed. No language in the specification should be construed as indicating any non-claimed element as essential to the practice of the disclosure.

The term “about,” as used herein, includes the recited number±10%. Thus, “about 10” means 9 to 11.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. However, in one embodiment, administration of Compound 1, Compound 2, Compound 3, or DON, and an immune checkpoint inhibitor leads to remission of the cancer.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, in one embodiment, a therapeutically effective amount will refer to the amount of a therapeutic agent that causes a therapeutic response, e.g., normalization of blood counts, decrease in the rate of tumor growth, decrease in tumor mass, decrease in the number of metastases, increase in time to tumor progression, and/or increase subject survival time by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more.

The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, Pa., 19th ed. 1995.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

In some embodiments, when administered in combination, two or more agents can have a synergistic effect. The terms “synergy,” “synergistic,” “synergistically” and derivations thereof, such as in a “synergistic effect” or a “synergistic combination” or a “synergistic composition” as used herein refer to circumstances under which the biological activity of a combination of an agent and at least one additional therapeutic agent is greater than the sum of the biological activities of the respective agents when administered individually. For example, the term “synergistically effective” as used herein refers to the interaction between Compound 1, Compound 2, Compound 3, or DON, and the immune checkpoint inhibitor that causes the total effect of the drugs to be greater than the sum of the individual effects of each drug. See, e.g, Berenbaum, Pharmacological Reviews 41:93-141 (1989).

Synergy can be expressed in terms of a “Synergy Index (SI),” which generally can be determined by the method described by F. C. Kull et al. Applied Microbiology 9, 538 (1961), from the ratio determined by:

$${Q}_{a} {Q}_{A} + {Q}_{b} {Q}_{B} = Synergy Index ( SI )$$

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

The terms “intermittent dose administration,” “intermittent dosing schedule,” and similar terms as used herein refer to, i.e., not continuous, administration, of Compound 1, Compound 2, Compound 3, or DON to a subject. Applicant has unexpectedly found that intermittent dose administration of Compound 1, Compound 2, Compound 3, or DON maintains or improves the anti-cancer efficacy achieved with continuous dosing, but with less side-effects, e.g., less body weight loss, in preclinical animal models. Intermittent dose administration regimens useful in the present disclosure encompass any discontinuous administration regimen that provides a therapeutically effective amount of Compound 1, Compound 2, Compound 3, or DON to a subject in need thereof. Intermittent dosing regimens can use equivalent, lower, or higher doses of Compound 1, Compound 2, Compound 3, or DON than would be used in continuous dosing regimens. Advantages of intermittent dose administration of Compound 1, Compound 2, Compound 3, or DON include, but are not limited to, improved safety, decreased toxicity, e.g., decreased weight loss, increased exposure, increased efficacy, and/or increased subject compliance. These advantages may be realized when Compound 1, Compound 2, Compound 3, or DON are administered as a single agent or when administered in combination with an immune checkpoint inhibitor and, optionally, one or more additional therapeutic agents. On the day Compound 1, Compound 2, Compound 3, or DON is scheduled to be administered to the subject, administration can occur in a single or in divided doses, e.g., once-a-day, twice-a-day, three times a day, four times a day or more. Dosing can also occur via any suitable route, e.g., orally or subcutaneously. In one embodiment, Compound 1, Compound 2, Compound 3, or DON is administered to the subject subcutaneously. In one embodiment, Compound 1, Compound 2, Compound 3, or DON is administered to the subject intravenously. In another embodiment, Compound 1, Compound 2, Compound 3, or DON is administered to the subject once (QD) or twice (BID) on the day the compound is scheduled to be administered.

In one embodiment, Compound 1, Compound 2, Compound 3, or DON is administered to the subject according to an intermittent dosing schedule to treat cancer. In another embodiment, the intermittent dosing schedule increases the therapeutic index of Compound 1, Compound 2, Compound 3, or DON. The therapeutic index is a comparison of the amount of Compound 1, Compound 2, Compound 3, or DON that causes the therapeutic effect, e.g., decrease in tumor mass, increase in time to tumor progression, and/or increase in subject survival time, to the amount that causes toxicity, e.g. body weight loss.

In one embodiment, Compound 1, Compound 2, Compound 3, or DON is administered to the subject every other day.

In another embodiment, Compound 1, Compound 2, Compound 3, or DON is administered to the subject once a week.

In another embodiment, Compound 1, Compound 2, Compound 3, or DON is administered to the subject twice a week on consecutive days, e.g., on Monday and Tuesday.

In another embodiment, Compound 1, Compound 2, Compound 3, or DON is administered to the subject twice a week on non-consecutive days, e.g., on Monday and Wednesday.

In another embodiment, Compound 1, Compound 2, Compound 3, or DON is administered to the subject three times a week on consecutive days, e.g., on Monday, Tuesday, and Wednesday.

In another embodiment, Compound 1, Compound 2, Compound 3, or DON is administered to the subject three times a week on non-consecutive days, e.g., on Monday, Wednesday, and Friday.

In one embodiment, Compound 1, Compound 2, Compound 3, or DON is administered to the subject for about consecutive 4 weeks in a row followed by 1 day or 2, 3, 4, 5, 6, or 7 consecutive days in a row wherein the compound is not administered to the subject.

In one embodiment, Compound 1, Compound 2, Compound 3, or DON is administered to the subject for about 3 consecutive weeks in a row followed by 1 day or 2, 3, 4, 5, 6, or 7 consecutive days in a row wherein the compound is not administered to the subject.

In one embodiment, Compound 1, Compound 2, Compound 3, or DON is administered to the subject for about 2 consecutive weeks in a row followed by 1 day or 2, 3, 4, 5, 6, or 7 consecutive days in a row wherein the compound is not administered to the subject.

In one embodiment, Compound 1, Compound 2, Compound 3, or DON is administered to the subject for 3 consecutive weeks in a row followed by 1 day or 2, 3, 4, or 5 consecutive days in a row wherein the compound is not administered to the subject

In one embodiment, Compound 1, Compound 2, Compound 3, or DON is administered to the subject for 2, 3, 4, 5, 6, 7, 8, 9, 10, 11, 12, 13, or 14 consecutive days in a row followed by 1 day or 2, 3, 4, or 5 consecutive days in a row wherein the compound is not administered to the subject.

In one embodiment, Compound 1, Compound 2, Compound 3, or DON is administered to the subject for 2, 3, 4, 5, 6, 7, 8, 9, 10, 11, 12, 13, or 14 consecutive days in a row followed by 1 day or 2, 3, or 4 consecutive days in a row wherein the compound is not administered to the subject.

In one embodiment, Compound 1, Compound 2, Compound 3, or DON is administered to the subject for 2, 3, 4, 5, 6, 7, 8, 9, or 10 consecutive days in a row followed by 1 day or 2, 3, or 4 consecutive days in a row wherein the compound is not administered to the subject.

In another embodiment, Compound 1, Compound 2, Compound 3, or DON is administered to the subject for 2 consecutive days in a row followed by 3 or 4 consecutive days in a row wherein the compound is not administered.

In another embodiment, Compound 1, Compound 2, Compound 3, or DON is administered to the subject for 3 consecutive days in a row followed by 3 or 4 days in a row wherein the compound is not administered.

In another embodiment, Compound 1, Compound 2, Compound 3, or DON is administered to the subject for 4 consecutive days in a row followed by 3 or 4 consecutive days in a row wherein the compound is not administered.

In another embodiment, Compound 1, Compound 2, Compound 3, or DON is administered to the subject for 5 consecutive days in a row followed by 3 or 4 consecutive days in a row wherein the compound is not administered.

In another embodiment, Compound 1, Compound 2, Compound 3, or DON is administered to the subject for 6 consecutive days in a row followed by 3 or 4 consecutive days in a row wherein the compound is not administered.

In another embodiment, Compound 1, Compound 2, Compound 3, or DON is administered to the subject for 7 consecutive days in a row followed by 3 or 4 consecutive days in a row wherein the compound is not administered.

In another embodiment, Compound 1, Compound 2, Compound 3, or DON is administered to the subject for 8 consecutive days in a row followed by 3 or 4 consecutive days in a row wherein the compound is not administered.

In another embodiment, Compound 1, Compound 2, Compound 3, or DON is administered to the subject for 9 consecutive days in a row followed by 3 or 4 consecutive days in a row wherein the compound is not administered.

In another embodiment, Compound 1, Compound 2, Compound 3, or DON is administered to the subject for 10 consecutive days in a row followed by 3 or 4 consecutive days in a row wherein the compound is not administered.

In another embodiment, Compound 1, Compound 2, Compound 3, or DON is administered to the subject for 11 consecutive days in a row followed by 3 or 4 consecutive days in a row wherein the compound is not administered.

In another embodiment, Compound 1, Compound 2, Compound 3, or DON is administered to the subject for 12 consecutive days in a row followed by 3 or 4 consecutive days in a row wherein the compound is not administered.

In another embodiment, Compound 1, Compound 2, Compound 3, or DON is administered to the subject for 13 consecutive days in a row followed by 3 or 4 consecutive days in a row wherein the compound is not administered.

In another embodiment, Compound 1, Compound 2, Compound 3, or DON is administered to the subject for 14 consecutive days in a row followed by 3 or 4 consecutive days in a row wherein the compound is not administered.

In another embodiment, Compound 1, Compound 2, Compound 3, or DON is administered to the subject for 15 consecutive days in a row followed by 3 or 4 consecutive days in a row wherein the compound is not administered.

In another embodiment, Compound 1, Compound 2, Compound 3, or DON is administered to the subject for 16 consecutive days in a row followed by 3 or 4 consecutive days in a row wherein the compound is not administered.

In another embodiment, Compound 1, Compound 2, Compound 3, or DON is administered to the subject for 17 consecutive days in a row followed by 3 or 4 consecutive days in a row wherein the compound is not administered.

In another embodiment, Compound 1, Compound 2, Compound 3, or DON is administered to the subject for 18 consecutive days in a row followed by 3 or 4 consecutive days in a row wherein the compound is not administered.

In another embodiment, Compound 1, Compound 2, Compound 3, or DON is administered to the subject for 19 consecutive days in a row followed by 3 or 4 consecutive days in a row wherein the compound is not administered.

In another embodiment, Compound 1, Compound 2, Compound 3, or DON is administered to the subject for 20 consecutive days in a row followed by 3 or 4 consecutive days in a row wherein the compound is not administered.

In another embodiment, Compound 1, Compound 2, Compound 3, or DON is administered to the subject for 21 consecutive days in a row followed by days 3 or 4 consecutive in a row wherein the compound is not administered.

In another embodiment, Compound 1, Compound 2, Compound 3, or DON is administered to the subject for 2 consecutive days in a row followed by 2 or 3 consecutive days in a row wherein the compound is not administered.

In another embodiment, Compound 1, Compound 2, Compound 3, or DON is administered to the subject for 3 consecutive days in a row followed by 2 or 3 consecutive days in a row wherein the compound is not administered.

In another embodiment, Compound 1, Compound 2, Compound 3, or DON is administered to the subject for 4 consecutive days in a row followed by 2 or 3 consecutive days in a row wherein the compound is not administered.

In another embodiment, Compound 1, Compound 2, Compound 3, or DON is administered to the subject for 5 consecutive days in a row followed by 2 or 3 consecutive days in a row wherein the compound is not administered.

In another embodiment, Compound 1, Compound 2, Compound 3, or DON is administered to the subject for 6 consecutive days in a row followed by 2 or 3 consecutive days in a row wherein the compound is not administered.

In another embodiment, Compound 1, Compound 2, Compound 3, or DON is administered to the subject for 7 consecutive days in a row followed by 2 or 3 consecutive days in a row wherein the compound is not administered.

In another embodiment, Compound 1, Compound 2, Compound 3, or DON is administered to the subject for 8 consecutive days in a row followed by 2 or 3 consecutive days in a row wherein the compound is not administered.

In another embodiment, Compound 1, Compound 2, Compound 3, or DON is administered to the subject for 9 consecutive days in a row followed by 2 or 3 consecutive days in a row wherein the compound is not administered.

In another embodiment, Compound 1, Compound 2, Compound 3, or DON is administered to the subject for 10 consecutive days in a row followed by 2 or 3 consecutive days in a row wherein the compound is not administered.

In another embodiment, Compound 1, Compound 2, Compound 3, or DON is administered to the subject for 11 consecutive days in a row followed by 2 or 3 consecutive days in a row wherein the compound is not administered.

In another embodiment, Compound 1, Compound 2, Compound 3, or DON is administered to the subject for 12 consecutive days in a row followed by 2 or 3 consecutive days in a row wherein the compound is not administered.

In another embodiment, Compound 1, Compound 2, Compound 3, or DON is administered to the subject for 13 consecutive days in a row followed by 2 or 3 consecutive days in a row wherein the compound is not administered.

In another embodiment, Compound 1, Compound 2, Compound 3, or DON is administered to the subject for 14 consecutive days in a row followed by 2 or 3 consecutive days in a row wherein the compound is not administered.

In another embodiment, Compound 1, Compound 2, Compound 3, or DON is administered to the subject for 15 consecutive days in a row followed by 2 or 3 consecutive days in a row wherein the compound is not administered.

In another embodiment, Compound 1, Compound 2, Compound 3, or DON is administered to the subject for 16 consecutive days in a row followed by 2 or 3 consecutive days in a row wherein the compound is not administered.

In another embodiment, Compound 1, Compound 2, Compound 3, or DON is administered to the subject for 17 consecutive days in a row followed by 2 or 3 consecutive days in a row wherein the compound is not administered.

In another embodiment, Compound 1, Compound 2, Compound 3, or DON is administered to the subject for 18 consecutive days in a row followed by 2 or 3 consecutive days in a row wherein the compound is not administered.

In another embodiment, Compound 1, Compound 2, Compound 3, or DON is administered to the subject for 19 consecutive days in a row followed by 2 or 3 consecutive days in a row wherein the compound is not administered.

In another embodiment, Compound 1, Compound 2, Compound 3, or DON is administered to the subject for 20 consecutive days in a row followed by 2 or 3 consecutive days in a row wherein the compound is not administered.

In another embodiment, Compound 1, Compound 2, Compound 3, or DON is administered to the subject for 21 consecutive days in a row followed by 2 or 3 consecutive days in a row wherein the compound is not administered.

In another embodiment, Compound 1, Compound 2, Compound 3, or DON is administered to the subject for 2 consecutive days in a row followed by 1 day or 2 consecutive days in a row wherein the compound is not administered.

In another embodiment, Compound 1, Compound 2, Compound 3, or DON is administered to the subject for 3 consecutive days in a row followed by 1 day or 2 consecutive days in a row wherein the compound is not administered.

In another embodiment, Compound 1, Compound 2, Compound 3, or DON is administered to the subject for 4 consecutive days in a row followed by 1 day or 2 consecutive days in a row wherein the compound is not administered.

In another embodiment, Compound 1, Compound 2, Compound 3, or DON is administered to the subject for 5 consecutive days in a row followed by 1 day or 2 consecutive days in a row wherein the compound is not administered.

In another embodiment, Compound 1, Compound 2, Compound 3, or DON is administered to the subject for 5 consecutive days in a row followed by 2 consecutive days in a row wherein the compound is not administered.

In another embodiment, Compound 1, Compound 2, Compound 3, or DON is administered to the subject for 6 consecutive days in a row followed by 1 day or 2 consecutive days in a row wherein the compound is not administered.

In another embodiment, Compound 1, Compound 2, Compound 3, or DON is administered to the subject for 7 consecutive days in a row followed by 1 day or 2 consecutive days in a row wherein the compound is not administered.

In another embodiment, Compound 1, Compound 2, Compound 3, or DON is administered to the subject for 8 consecutive days in a row followed by 1 day or 2 consecutive days in a row wherein the compound is not administered.

In another embodiment, Compound 1, Compound 2, Compound 3, or DON is administered to the subject for 9 consecutive days in a row followed by 1 day or 2 consecutive days in a row wherein the compound is not administered.

In another embodiment, Compound 1, Compound 2, Compound 3, or DON is administered to the subject for 10 consecutive days in a row followed by 1 day or 2 consecutive days in a row wherein the compound is not administered.

In another embodiment, Compound 1, Compound 2, Compound 3, or DON is administered to the subject for 11 consecutive days in a row followed by 1 day or 2 consecutive days in a row wherein the compound is not administered.

In another embodiment, Compound 1, Compound 2, Compound 3, or DON is administered to the subject for 12 consecutive days in a row followed by 1 day or 2 consecutive days in a row wherein the compound is not administered.

In another embodiment, Compound 1, Compound 2, Compound 3, or DON is administered to the subject for 13 consecutive days in a row followed by 1 day or 2 consecutive days in a row wherein the compound is not administered.

In another embodiment, Compound 1, Compound 2, Compound 3, or DON is administered to the subject for 14 consecutive days in a row followed by 1 day or 2 consecutive days in a row wherein the compound is not administered.

In another embodiment, Compound 1, Compound 2, Compound 3, or DON is administered to the subject for 15 consecutive days in a row followed by 1 day or 2 consecutive days in a row wherein the compound is not administered.

In another embodiment, Compound 1, Compound 2, Compound 3, or DON is administered to the subject for 16 consecutive days in a row followed by 1 day or 2 consecutive days in a row wherein the compound is not administered.

In another embodiment, Compound 1, Compound 2, Compound 3, or DON is administered to the subject for 17 consecutive days in a row followed by 1 day or 2 consecutive days in a row wherein the compound is not administered.

In another embodiment, Compound 1, Compound 2, Compound 3, or DON is administered to the subject for 18 consecutive days in a row followed by 1 day or 2 consecutive days in a row wherein the compound is not administered.

In another embodiment, Compound 1, Compound 2, Compound 3, or DON is administered to the subject for 19 consecutive days in a row followed by 1 day or 2 consecutive days in a row wherein the compound is not administered.

In another embodiment, Compound 1, Compound 2, Compound 3, or DON is administered to the subject for 20 consecutive days in a row followed by 1 day or 2 consecutive days in a row wherein the compound is not administered.

In another embodiment, Compound 1, Compound 2, Compound 3, or DON is administered to the subject for 21 consecutive days in a row followed by 1 day or 2 consecutive days in a row wherein the compound is not administered.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to an individual in a sequence and sufficiently close in time so as to provide the desired therapeutic effect and can act in concert. For example, Compound 1, Compound 2, Compound 3, or DON can be administered at the same time or sequentially in any order at different points in time as the immune checkpoint inhibitor. Compound 1, Compound 2, Compound 3, or DON, and the immune checkpoint inhibitor can be administered separately, in any appropriate form and by any suitable route, e.g., by SC and by IV injection, respectively. When Compound 1, Compound 2, Compound 3, or DON, and the immune checkpoint inhibitor are not administered concurrently, it is understood that they can be administered in any order to a subject in need thereof. For example, Compound 1, Compound 2, Compound 3, or DON can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, or more before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, or more after) the immune checkpoint inhibitor. In various embodiments, Compound 1, Compound 2, Compound 3, or DON, and the immune checkpoint inhibitor are administered 1 minute apart, 10 minutes apart, 30 minutes apart, less than 1 hour apart, about 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart, no more than 48 hours apart, no more than 3 days apart, or no more than 1 week apart. In one embodiment, Compound 1, Compound 2, Compound 3, or DON is administered 1-14 days prior to the day the immune checkpoint inhibitor is administered. In one embodiment, Compound 1, Compound 2, Compound 3, or DON is administered 1-7 days prior to the day the immune checkpoint inhibitor is administered. In another embodiment, Compound 1, Compound 2, Compound 3, or DON is also administered on the day the immune checkpoint inhibitor is administered.

### VI. Particular Embodiments

The disclosure provides the following particular embodiments.

Embodiment 1. A method of treating a subject having cancer, the method comprising administering to the subject in need thereof a therapeutically effective amount of:

(a) Compound 1; or

(b) Compound 2; or

(c) Compound 3; or

(c) DON; and

(d) an immune checkpoint inhibitor,

wherein Compound 1 or Compound 2 or Compound 3 or DON is administered to the subject according to an intermittent dosing schedule.

Embodiment 2. The method of Embodiment 1, wherein immune checkpoint inhibitor is selected from the group consisting of a PD-1 inhibitor, a PD-L1 inhibitor, a CTLA-4 inhibitor, a LAG3 inhibitor, and a TIM3 inhibitor.

Embodiment 3. The method of Embodiment 2, wherein the immune checkpoint inhibitor is a PD-1 inhibitor.

Embodiment 4. The method of Embodiment 3, wherein the PD-1 inhibitor is an anti-PD-1 antibody.

Embodiment 5. The method of Embodiment 4, wherein the anti-PD-1 antibody is selected from the group consisting of nivolumab, pembrolizumab, pidilizumab, STI-A1110, PDR001, MEDI-0680, AGEN2034, BGB-A317, AB122, TSR-042, PF-06801591, cemiplimab, SYM021, JNJ-63723283, HLX10, LZM009, and MGA012.

Embodiment 6. The method of Embodiment 2, wherein the immune checkpoint inhibitor is a PD-L1 inhibitor.

Embodiment 7. The method of Embodiment 6, wherein the PD-L1 inhibitor is an anti-PD-L1 antibody.

Embodiment 8. The method of Embodiment 7, wherein the anti-PD-L1 antibody is selected from the group consisting of avelumab, atezolizumab, durvalumab, and STI-A1014.

Embodiment 9. The method of Embodiment 2, wherein the immune checkpoint inhibitor is an anti-CTLA-4 inhibitor.

Embodiment 10. The method of Embodiment 9, wherein the anti-CTLA-4 inhibitor is an anti-CTLA-4 antibody.

Embodiment 11. The method of Embodiment 10, wherein the anti-CTLA-4 antibody is selected from the group consisting of ipilimumab and tremelimumab.

Embodiment 12. The method of Embodiment 2, wherein the immune checkpoint inhibitor is a LAG3 inhibitor.

Embodiment 13. The method of Embodiment 12, wherein the LAG3 inhibitor is an anti-LAG3 antibody.

Embodiment 14. The method of Embodiment 13, wherein the anti-LAG3 antibody is GSK2831781.

Embodiment 15. The method of Embodiment 2, wherein the immune checkpoint inhibitor is a TIM3 inhibitor.

Embodiment 16. The method of Embodiment 15, wherein the TIM3 inhibitor is an anti-TIM3 antibody.

Embodiment 17. The method of any one of Embodiments 1-16, wherein the cancer has become resistant to treatment with at least one immune checkpoint inhibitor.

Embodiment 18. The method of any one of Embodiments 1-17, wherein Compound 1 or Compound 2 or DON is administered to the subject before the immune checkpoint inhibitor.

Embodiment 19. The method of any one of Embodiments 1-17, wherein Compound 1 or Compound 2 or DON is administered to the subject after the immune checkpoint inhibitor.

Embodiment 20. The method of any one of Embodiments 1-17, wherein Compound 1 or Compound 2 or DON is administered to the subject at the same time as the immune checkpoint inhibitor.

Embodiment 21. The method of any one of Embodiments 1-20, wherein the administration of Compound 1 or Compound 2 or DON, and the immune checkpoint inhibitor to the subject is synergistically effective to treat cancer in the subject.

Embodiment 22. The method of any one of Embodiments 1-21, wherein the cancer is a solid tumor.

Embodiment 23. The method of any one of Embodiments 1-21, wherein the cancer is a hematological cancer.

Embodiment 24. The method of any one of Embodiments 1-21, wherein the cancer selected from the group of cancers listed in Table 1.

Embodiment 25. The method of Embodiment 24, wherein the cancer is selected from the group consisting of hepatocellular carcinoma, glioblastoma, lung cancer, breast cancer, head and neck cancer, prostate cancer, melanoma, and colorectal cancer.

Embodiment 26. The method of Embodiment 26, wherein the cancer is colorectal cancer, breast cancer, lymphoma, melanoma, kidney cancer, and lung cancer.

Embodiment 27. The method of any one of Embodiments 1-26, wherein Compound 1 or Compound 2 or Compound 3 or DON is administered to the subject for 3, 4, 5, 6, 7, 8, 9, or consecutive 10 days in a row followed by 2 consecutive days in a row wherein Compound 1 or Compound 2 or Compound 3 or DON is not administered to the subject.

Embodiment 28. The method of Embodiment 27, wherein Compound 1 or Compound 2 or Compound 3 or DON is administered to the subject for 5 consecutive days in a row followed by 2 consecutive days in a row wherein Compound 1 or Compound 2 or DON is not administered to the subject.

Embodiment 29. The method of any one of Embodiments 1-28, wherein Compound 1 or Compound 2 or Compound 3 or DON is subcutaneously administered to the subject.

Embodiment 30. The method of any one of Embodiments 1-29, wherein DON is administered to the subject.

Embodiment 31. A method of treating a subject having cancer, the method comprising administering to the subject in need thereof a therapeutically effective amount of Compound 1 or Compound 2 according to an intermittent dosing schedule.

Embodiment 32. The method of Embodiment 31, wherein the cancer is a solid tumor.

Embodiment 33. The method of Embodiment 31, wherein the cancer is a hematological cancer.

Embodiment 34. The method of Embodiment 31, wherein the cancer selected from the group of cancers listed in Table 1.

Embodiment 35. The method of Embodiment 34, wherein the cancer is selected from the group consisting of hepatocellular carcinoma, glioblastoma, lung cancer, breast cancer, head and neck cancer, prostate cancer, melanoma, and colorectal cancer.

Embodiment 36. The method of Embodiment 35, wherein the cancer is colorectal cancer, breast cancer, lymphoma, melanoma, kidney cancer, and lung cancer.

Embodiment 37. The method of any one of Embodiments 31-36, wherein Compound 1 or Compound 2 or Compound 3 is administered to the subject for 3, 4, 5, 6, 7, 8, 9, or 10 consecutive days in a row followed by 2 consecutive days in a row wherein Compound 1 or Compound 2 or Compound 3 is not administered to the subject.

Embodiment 38. The method of Embodiment 37, wherein Compound 1 or Compound 2 or Compound 3 is administered to the subject for 5 consecutive days in a row followed by 2 consecutive days in a row wherein Compound 1 or Compound 2 or Compound 3 is not administered to the subject.

Embodiment 39. The method of any one of Embodiments 31-38, wherein Compound 1 or Compound 2 or Compound 3 is subcutaneously administered to the subject.

Embodiment 40. The method of any one of Embodiments 1-39, wherein Compound 1 is administered to the subject.

Embodiment 41. The method of any one of Embodiments 1-39, wherein Compound 2 is administered to the subject.

Embodiment 42. The method of any one of Embodiments 1-41, wherein the subject is a human.

Embodiment 43. Compound 1 or Compound 2 or Compound 3 or DON, or a pharmaceutical composition comprising Compound 1 or Compound 2 or Compound 3 or DON, and a pharmaceutically acceptable excipient for use in treating cancer in a subject, wherein the compound or composition is administered to the subject according to an intermittent dosing schedule in combination with an immune checkpoint inhibitor.

Embodiment 44. The compound or composition for use of Embodiment 43, wherein the immune checkpoint inhibitor is selected from the group consisting of a PD-1 inhibitor, a PD-L1 inhibitor, a CTLA-4 inhibitor, a LAG3 inhibitor, and a TIM3 inhibitor.

Embodiment 45. The compound or composition for use of Embodiment 44, wherein the immune checkpoint inhibitor is a PD-1 inhibitor.

Embodiment 46. The compound or composition for use of Embodiment 45, wherein the PD-1 inhibitor is an anti-PD-1 antibody.

Embodiment 47. The compound or composition for use of Embodiment 46, wherein the anti-PD-1 antibody is selected from the group consisting of nivolumab, pembrolizumab, pidilizumab, STI-A1110, PDR001, MEDI-0680, AGEN2034, BGB-A317, AB122, TSR-042, PF-06801591, cemiplimab, SYM021, JNJ-63723283, HLX10, LZM009, and MGA012.

Embodiment 48. The compound or composition for use of Embodiment 44, wherein the immune checkpoint inhibitor is a PD-L1 inhibitor.

Embodiment 49. The compound or composition for use of Embodiment 48, wherein the PD-L1 inhibitor is an anti-PD-L1 antibody.

Embodiment 50. The compound or composition for use of Embodiment 49, wherein the anti-PD-L1 antibody is selected from the group consisting of avelumab, atezolizumab, durvalumab, and STI-A1014.

Embodiment 51. The compound or composition for use of Embodiment 44, wherein the immune checkpoint inhibitor is an anti-CTLA-4 inhibitor.

Embodiment 52. The compound or composition for use of Embodiment 51, wherein the anti-CTLA-4 inhibitor is an anti-CTLA-4 antibody.

Embodiment 53. The compound or composition for use of Embodiment 52, wherein the anti-CTLA-4 antibody is selected from the group consisting of ipilimumab and tremelimumab.

Embodiment 54. The compound or composition for use of Embodiment 44, wherein the immune checkpoint inhibitor is a LAG3 inhibitor.

Embodiment 55. The compound or composition for use of Embodiment 54, wherein the LAG3 inhibitor is an anti-LAG3 antibody.

Embodiment 56. The compound or composition for use of Embodiment 55, wherein the anti-LAG3 antibody is GSK2831781.

Embodiment 57. The compound or composition for use of Embodiment 44, wherein the immune checkpoint inhibitor is a TIM3 inhibitor.

Embodiment 58. The compound or composition for use of Embodiment 57, wherein the TIM3 inhibitor is an anti-TIM3 antibody.

Embodiment 59. The compound or composition for use of any one of Embodiments 43-58, wherein the cancer is or has become resistant to treatment with at least one immune checkpoint inhibitor.

Embodiment 60. The compound or composition for use of any one of Embodiments 43-59, wherein the compound or composition is administered to the subject before the immune checkpoint inhibitor.

Embodiment 61. The compound or composition for use of any one of Embodiments 43-59, wherein the compound or composition is administered to the subject after the immune checkpoint inhibitor.

Embodiment 62. The Compound 1 or Compound 2 or DON for use of any one of Embodiments 43-59, wherein the compound or composition is administered to the subject at the same time as the immune checkpoint inhibitor.

Embodiment 63. The compound or composition for use of any one of Embodiments 43-62, wherein the administration of the compound or composition and the immune checkpoint inhibitor to the subject is synergistically effective to treat cancer in the subject.

Embodiment 64. Compound 1 or Compound 2 or Compound 3 or DON, or a pharmaceutical composition comprising Compound 1 or Compound 2 or Compound 3 or DON, and a pharmaceutically acceptable excipient for use in treating cancer in a subject, wherein the compound or composition is administered to the subject according to an intermittent dosing schedule.

Embodiment 65. The compound or composition for use of any one of Embodiments 43-64, wherein the cancer is a solid tumor.

Embodiment 66. The compound or composition for use of any one of Embodiments 43-64, wherein the cancer is a hematological cancer.

Embodiment 67. The compound or composition for use of any one of Embodiments 43-64, wherein the cancer selected from the group of cancers listed in Table 1.

Embodiment 68. The compound or composition for use of Embodiment 67, wherein the cancer is selected from the group consisting of hepatocellular carcinoma, glioblastoma, lung cancer, breast cancer, head and neck cancer, prostate cancer, melanoma, and colorectal cancer.

Embodiment 69. The compound or composition for use of Embodiment 68, wherein the cancer is selected from the group consisting of colorectal cancer, breast cancer, lymphoma, melanoma, kidney cancer, and lung cancer

Embodiment 70. The compound or composition for use of any one of Embodiments 43-60, wherein the compound or composition is administered to the subject for 3, 4, 5, 6, 7, 8, 9, or 10 consecutive days in a row followed by 2 consecutive days in a row wherein the compound or composition is not administered to the subject.

Embodiment 71. The compound or composition for use of Embodiment 70, wherein the compound or composition is administered to the subject for 5 consecutive days in a row followed by 2 consecutive days in a row wherein the compound or composition is not administered to the subject.

Embodiment 72. The compound or composition for use of any one of Embodiments 43-71, wherein the compound or composition is subcutaneously administered to the subject.

Embodiment 73. The compound or composition for use of any one of Embodiments 43-72, wherein Compound 1, or a pharmaceutical composition comprising Compound 1, and a pharmaceutically acceptable excipient is administered to the subject.

Embodiment 74. The compound or composition for use of any one of Embodiments 43-72, wherein Compound 2, or a pharmaceutical composition comprising Compound 2, and a pharmaceutically acceptable excipient is administered to the subject.

Embodiment 75. Use of Compound 1 or Compound 2 or Compound 3 or DON in the manufacture of a medicament for treating cancer in a subject, wherein the compound is administered to the subject according to an intermittent dosing schedule in combination with an immune checkpoint inhibitor.

Embodiment 76. The use of Embodiment 75, wherein the immune checkpoint inhibitor is selected from the group consisting of a PD-1 inhibitor, a PD-L1 inhibitor, a CTLA-4 inhibitor, a LAG3 inhibitor, and a TIM3 inhibitor.

Embodiment 77. The use of Embodiment 76, wherein the immune checkpoint inhibitor is a PD-1 inhibitor.

Embodiment 78. The use of Embodiment 77, wherein the PD-1 inhibitor is an anti-PD-1 antibody.

Embodiment 79. The use of Embodiment 78, wherein the anti-PD-1 antibody is selected from the group consisting of nivolumab, pembrolizumab, pidilizumab, STI-A1110, PDR001, MEDI-0680, AGEN2034, BGB-A317, AB122, TSR-042, PF-06801591, cemiplimab, SYM021, JNJ-63723283, HLX10, LZM009, and MGA012.

Embodiment 80. The use of Embodiment 76, wherein the immune checkpoint inhibitor is a PD-L1 inhibitor.

Embodiment 81. The use of Embodiment 80, wherein the PD-L1 inhibitor is an anti-PD-L1 antibody.

Embodiment 82. The use of Embodiment 81, wherein the anti-PD-L1 antibody is selected from the group consisting of avelumab, atezolizumab, durvalumab, and STI-AI014.

Embodiment 83. The use of Embodiment 76, wherein the immune checkpoint inhibitor is an anti-CTLA-4 inhibitor.

Embodiment 84. The use of Embodiment 83, wherein the anti-CTLA-4 inhibitor is an anti-CTLA-4 antibody.

Embodiment 85. The use of Embodiment 84, wherein the anti-CTLA-4 antibody is selected from the group consisting of ipilimumab and tremelimumab.

Embodiment 86. The use of Embodiment 76, wherein the immune checkpoint inhibitor is a LAG3 inhibitor.

Embodiment 87. The use of Embodiment 86, wherein the LAG3 inhibitor is an anti-LAG3 antibody.

Embodiment 88. The use of Embodiment 87, wherein the anti-LAG3 antibody is GSK2831781.

Embodiment 89. The use of Embodiment 76, wherein the immune checkpoint inhibitor is a TIM3 inhibitor.

Embodiment 90. The use of Embodiment 89, wherein the TIM3 inhibitor is an anti-TIM3 antibody.

Embodiment 91. The use of any one of Embodiments 75-90, wherein the cancer has become resistant to treatment with at least one immune checkpoint inhibitor.

Embodiment 92. The use of any one of Embodiments 75-91, wherein the compound is administered to the subject before the immune checkpoint inhibitor.

Embodiment 93. The use of any one of Embodiments 75-91, wherein the compound is administered to the subject after the immune checkpoint inhibitor.

Embodiment 94. The use of any one of Embodiments 75-91, wherein the compound is administered to the subject at the same time as the immune checkpoint inhibitor.

Embodiment 95. The use of any one of Embodiments 75-94, wherein the administration of the compound and the immune checkpoint inhibitor to the subject is synergistically effective to treat cancer in the subject.

Embodiment 96. Use of Compound 1 or Compound 2 or Compound 3 or DON in the manufacture of a medicament for treating cancer in a subject, wherein the compound is administered to the subject according to an intermittent dosing schedule.

Embodiment 97. The use of any one of Embodiments 75-96, wherein the cancer is a solid tumor.

Embodiment 98. The use of any one of Embodiments 75-96, wherein the cancer is a hematological cancer.

Embodiment 99. The use of any one of Embodiments 75-96, wherein the cancer selected from the group of cancers listed in Table 1.

Embodiment 100. The use of Embodiment 99, wherein the cancer is selected from the group consisting of hepatocellular carcinoma, glioblastoma, lung cancer, breast cancer, head and neck cancer, prostate cancer, melanoma, and colorectal cancer.

Embodiment 101. The use of Embodiment 100, wherein the cancer is selected from the group consisting of colorectal cancer, breast cancer, lymphoma, melanoma, kidney cancer, and lung cancer.

Embodiment 102. The use of any one of Embodiments 74-101, wherein the compound is administered to the subject for 3, 4, 5, 6, 7, 8, 9, or 10 consecutive days in a row followed by 2 consecutive days in a row wherein the compound is not administered to the subject.

Embodiment 103. The use of Embodiment 102, wherein the compound is administered to the subject for 5 consecutive days in a row followed by 2 consecutive days in a row wherein the compound is not administered to the subject.

Embodiment 104. The use of any one of Embodiments 75-103, wherein the compound is subcutaneously administered to the subject.

Embodiment 105. The use of any one of Embodiments 75-104, wherein Compound 1 is administered to the subject.

Embodiment 106. The use of any one of Embodiments 75-104, wherein Compound 2 is administered to the subject.

Embodiment 107. A method of treating a subject having cancer, the method comprising administering to the subject in need thereof a therapeutically effective amount of DON for 5 consecutive days followed by 2 consecutive days wherein DON is not administered.

Embodiment 108. The method of Embodiment 107, wherein the cancer is a solid tumor.

Embodiment 109. The method of Embodiment 107, wherein the cancer is a hematological cancer.

Embodiment 110. The method of Embodiment 107, wherein the cancer selected from the group of cancers listed in Table 1.

Embodiment 111. The method of Embodiment 110, wherein the cancer is selected from the group consisting of hepatocellular carcinoma, glioblastoma, lung cancer, breast cancer, head and neck cancer, prostate cancer, melanoma, and colorectal cancer.

Embodiment 112. The method of Embodiment 111, wherein the cancer is colorectal cancer, breast cancer, lymphoma, melanoma, kidney cancer, and lung cancer.

Embodiment 113. The method of any one of Embodiments 107-112, wherein DON is subcutaneously administered to the subject.

Embodiment 114. The method of any one of Embodiments 107-113, wherein the subject is a human.

Embodiment 115. The method of any one of Embodiments 107-114, wherein about 0.1 mg/kg to about 2 mg/kg of DON is administered to the subject.

Embodiment 116: The method of any one of Embodiments 1-28, wherein Compound 1 or Compound 2 or Compound 3 or DON is intravenously administered to the subject.

Embodiment 117: The method of any one of Embodiments 31-38, wherein Compound 1 or Compound 2 or Compound 3 is intravenously administered to the subject.

Embodiment 118. The method of any one of Embodiments 1-39, wherein Compound 3 is administered to the subject.

Embodiment 119. (S)-2-((S)-2-acetamido-3-(1H-indol-3-yl)propanamido)-6-diazo-5-oxohexanoic acid (Compound 3), or a pharmaceutically acceptable salt thereof.

Embodiment 120. A pharmaceutical composition comprising the compound of Embodiment 119, or a pharmaceutically acceptable salt thereof, and a pharmaceutically acceptable excipient.

### EXAMPLES

The following abbreviations may be used in the EXAMPLES:

### AAALAC Association for Assessment and Accreditation of Laboratory Animal Care

i.p. intraperitoneal

### IACUC Institutional Animal Care and Use Committee

### GLP Good Laboratory Practice

RT Room temperature

### QD Once per day

s.c. subcutaneous injection
BW Body weight
BWL Body weight loss
RCBW Relative change of body weight
TV Tumor volume
RTV Relative tumor volume
TGI Tumor growth inhibition
PBS Phosphate buffered saline
SEM Standard error of the mean
N Number of animals

### D Day

### EDTA Ethylene Diamine Tetraacetic Acid

DMEM Dulbecco's modified eagle medium

### G Group

No. number
g gram
mm³ cubic millimeter
mpk mg/kg

In the EXAMPLES, Compound 1 was administered as the free base and may be referred to in the tables and figures as “Cpd. 1.” In the combination studies comprising anti-PD-1, anti mPD-1 from BioXcell (catalog number BE0146) was used.

### Example 1

### In Vivo Test of Anti-Tumor Efficacy of Compound 1 in the Subcutaneous MC-38 Murine Colorectal Cancer Model in Female: Dose Response

### I. Abstract

Female C57BL/6 mice were inoculated subcutaneously at right flank with MC-38 cells for tumor development. Four days after tumor inoculation, 64 mice with tumor size ranging from 50-92 mm³ (average tumor size 63 mm³) were selected and assigned into 8 groups using stratified randomization with 8 mice per group based upon their tumor volumes. The treatments were started from the day of randomization (defined as D0), Group 1 was treated with Vehicle (Tween 80:Ethanol:Saline—5:5:90 v/v/v), S.C. QD, Group 2 was treated with Compound 1 (0.1 mg/kg), S.C. QD, Group 3 was treated with Compound 1 (0.3 mg/kg), S.C. QD, Group 4 was treated with Compound 1 (0.5 mg/kg), S.C. QD, Group 5 was treated with Compound 1 (1 mg/kg), S.C. QD, Group 6 was treated with Compound 1 (3 mg/kg), S.C. QD*5 days followed by Compound 1 (1 mg/kg), S.C. QD*9 days (2 cycles), Group 7 was treated with Compound 1 (1 mg/kg), S.C. QD*5 days followed by Compound 1 (0.3 mg/kg), S.C. QD*9 days (2 cycles) and Group 8 was treated with Compound 1 (0.15 mg/kg), S.C. BID. The tumor sizes were measured three times per week during the treatment. The entire study was terminated on D74 after start of the treatment.

### II. Experimental Methods and Procedures

Animal Species: Mus musculus; Strain: C57BL/6; Age: 6-8 weeks; Sex: female; Body weight (at treatment start): 17-21 g.

The MC-38 tumor cells were maintained in vitro as a monolayer culture in DMEM medium supplemented with 10% heat inactivated fetal bovine serum and 100 μg/mL penicillin streptomycin at 37° C. in an atmosphere of 5% CO₂ in air. The tumor cells were routinely subcultured twice weekly by trypsin-EDTA treatment. The cells growing to a confluency around 70%-80% were harvested and counted for tumor inoculation. The cultured MC-38 were harvested, re-suspended in base medium at a density of 1×10⁷ cells/mL with viability>90%. Each mouse was inoculated subcutaneously at the right flank with 1×10⁶ in 0.1 ml base medium for tumor development.

The treatments were started on day 4 after tumor inoculation when the tumor size reached 50-92 mm³ (average tumor size 63 mm³). Each group consisted of 8 tumor bearing mice. The testing article was administrated at a dosing volume of 10 mg/kg to the mice according to the predetermined regimen shown in Table 1-1. Testing article formulations were prepared according to standard procedures. Details regarding tumor measurements and endpoints, and statistical analysis are provided in EXAMPLE 3.

TABLE 1-1
Groups and Treatments for Efficacy Study

| | | Dose | |
|---|---|---|---|
| Group | Treatment | (mg/kg) | Dosing Route & Schedule |
| 1 | Vehicle control | — | QDx18, S.C. |
| 2 | Compound 1 | 0.1 | QDx28, S.C. |
| 3 | Compound 1 | 0.3 | QDx28, S.C. |
| 4 | Compound 1 | 0.5 | QDx28, S.C. |
| 5 | Compound 1 | 1.0 | QDx28, S.C. |
| 6 | Compound 1 | 3.0 | QDx5, S.C. followed by 1 |
| | | | mg/kg, QDx9, S.C. (2 cycles) |
| 7 | Compound 1 | 1.0 | QDx5, S.C. followed by 0.3 |
| | | | mg/kg, QDx9, S.C. (2 cycles) |
| 8 | Compound 1 | 0.15 | BID x28, S.C. |

### III. Results

### Body Weight Change

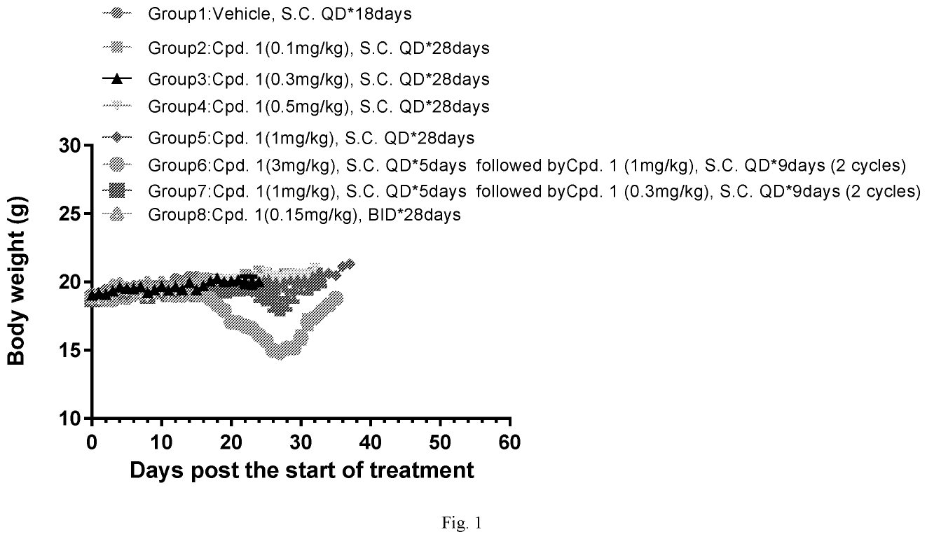

Group 6 showed some body weight loss but the other treatments were well-tolerated without any adverse effect observed by the MC-38 tumor bearing C57BL/6 mice. Body weight change in female C57BL/6 mice bearing MC-38 tumors are shown in FIG. 1.

### Tumor Growth Analysis

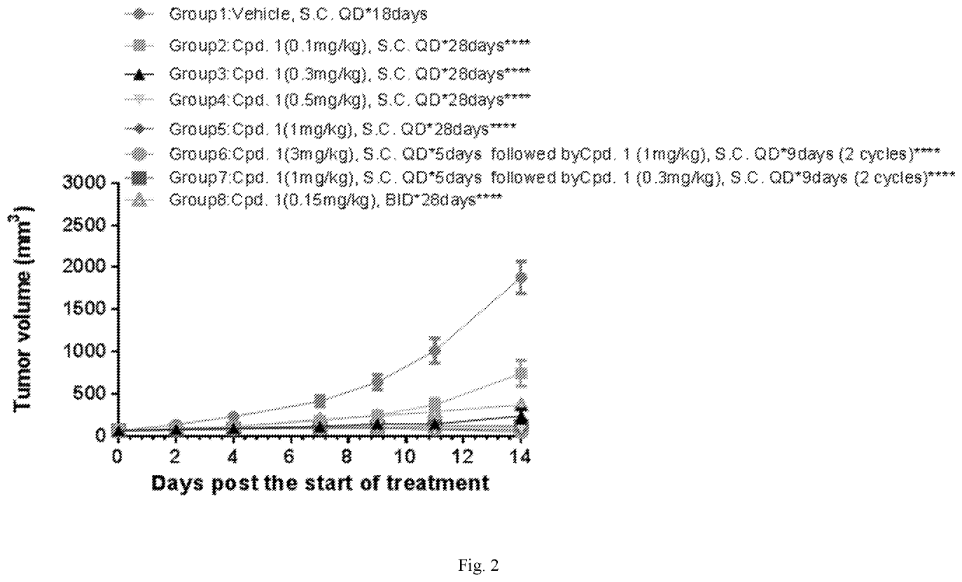

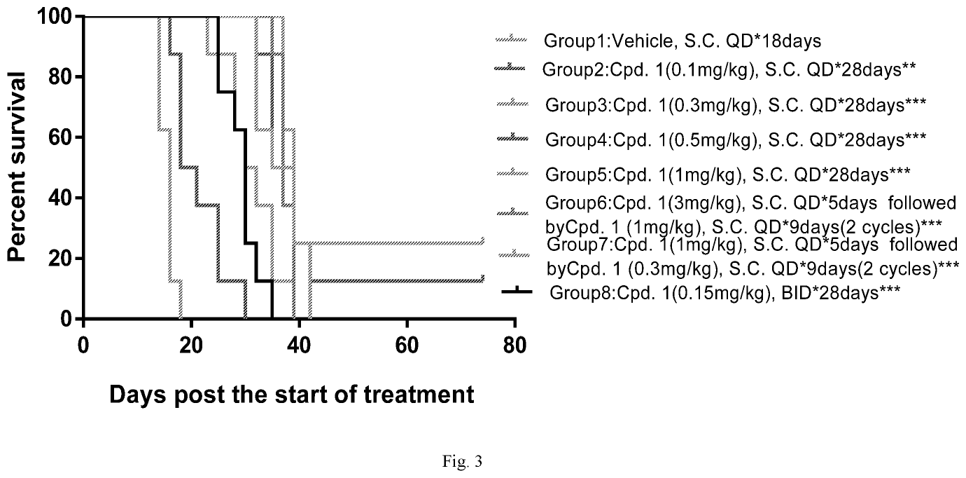

Mean tumor volume over time in female C57BL/6 mice bearing MC-38 tumors dosed with Compound 1 is shown in Table 1-2 and FIG. 2. The tumor growth inhibition analysis is shown in Table 1-3. The Kaplan-Meier survival curve (endpoint is defined as tumor volume reaching 2000 mm³) is shown in FIG. 3. The survival analysis is shown in Table 1-4.

Time-to-endpoint Kaplan-Meier survival analyses showed that all treatment groups showed significant survival benefit when compared to the vehicle group. One animal in group 4 and two animals in group 7 had complete rejection of the tumor and remained tumor-free at the termination day.

TABLE 1-2
Tumor Volume over Time

| | Tumor volume (mm³)^{a} | | | | | | | |
|---|---|---|---|---|---|---|---|---|
| | | Compound 1 (mg/Kg) | | | | | | |
| Days | Vehicle | 0.1 | 0.3 | 0.5 | 1 | 3 & 1 | 1 & 0.3 | 0.15 |
| 0 | 62 ± 4 | 62 ± 4 | 62 ± 4 | 63 ± 5 | 62 ± 4 | 63 ± 5 | 63 ± 4 | 63 ± 5 |
| 2 | 135 ± 18 | 91 ± 10 | 76 ± 5 | 79 ± 4 | 74 ± 5 | 70 ± 3 | 73 ± 5 | 87 ± 6 |
| 4 | 231 ± 29 | 112 ± 11 | 88 ± 7 | 97 ± 8 | 85 ± 5 | 83 ± 7 | 85 ± 7 | 109 ± 6 |
| 7 | 412 ± 63 | 183 ± 32 | 110 ± 14 | 112 ± 8 | 94 ± 5 | 87 ± 8 | 97 ± 10 | 195 ± 28 |
| 9 | 639 ± 88 | 247 ± 40 | 143 ± 26 | 109 ± 7 | 100 ± 7 | 90 ± 6 | 101 ± 9 | 237 ± 30 |
| 11 | 1011 ± 147 | 368 ± 72 | 140 ± 29 | 84 ± 9 | 80 ± 8 | 79 ± 10 | 116 ± 19 | 288 ± 43 |
| 14 | 1881 ± 190 | 744 ± 154 | 235 ± 80 | 97 ± 17 | 68 ± 12 | 43 ± 4 | 119 ± 22 | 369 ± 56 |

^{a}Mean ± SEM;
n = 8

TABLE 1-3
Tumor Growth Inhibition Calculation Based on TV Measurements at Day 14

| | Tumor Size | | | | |
|---|---|---|---|---|---|
| Treatment | (mm³) | RTV | T/C | | |
| (mg/kg) | at day 14 | at day 14 | (%) | TGI (%) | p value |
| Vehicle | 1881.36 ± 189.57 | 30.52 ± 6.9 | — | — | — |
| Cpd. 1 (0.1) | 743.79 ± 153.64 | 12.29 ± 7.63 | 40.26 | 62.52 | <0.001 |
| Cpd. 1 (0.3) | 235.18 ± 79.89 | 3.83 ± 3.95 | 12.54 | 90.47 | <0.001 |
| Cpd. 1 (0.5) | 96.6 ± 16.6 | 1.63 ± 0.88 | 5.33 | 98.16 | <0.001 |
| Cpd. 1 (1) | 68.23 ± 12.12 | 1.13 ± 0.67 | 3.71 | 99.69 | <0.001 |
| Cpd. 1 (3 & 1) | 42.91 ± 3.89 | 0.69 ± 0.16 | 2.27 | 101.09 | <0.001 |
| Cpd. 1 (1 & 0.3) | 118.69 ± 21.92 | 1.85 ± 0.82 | 6.05 | 96.95 | <0.001 |
| Cpd. 1 (0.1) | 369.41 ± 56.37 | 5.99 ± 2.92 | 19.62 | 83.19 | <0.001 |

TABLE 1-4
Survival Analysis

| | MST | ILS | |
|---|---|---|---|
| Treatment | (day) | (%) | p value^{a} |
| Vehicle | 16 | — | — |
| Compound 1 (0.1 mg/kg) | 19.5 | 22% | <0.01 |
| Compound 1 (0.3 mg/kg) | 31 | 94% | <0.001 |
| Compound 1 (0.5 mg/kg) | 37 | 131% | <0.001 |
| Compound 1 (1 mg/kg) | 39 | 144% | <0.001 |
| Compound 1 (3 mg/kg), Compound 1 (1 mg/kg) | 37 | 131% | <0.001 |
| Compound 1 (1 mg/kg), Compound 1 (0.3 mg/kg) | 37 | 131% | <0.001 |
| Compound 1 (0.15 mg/kg) | 30 | 88% | <0.001 |

### Example 2A

### In Vivo Test of Anti-Tumor Efficacy of Compound 1 with and without Anti-PD-1 in the 4T1 Murine Mammary Cancer Model in Female BALB/c Mice

### I. Abstract

Female BALB/c mice were inoculated subcutaneously at the mammary fat pat with 4T1 cells for tumor development. Six days after tumor inoculation, 48 mice with tumor size ranging from 49-88 mm³ (average tumor size 59 mm³) were selected and assigned into 6 groups using stratified randomization with 8 mice per group based upon their tumor volumes. The treatments were started from the day of randomization (defined as D0), Group 1 was treated with Vehicle (Tween 80:Ethanol:Saline—5:5:90 v/v/v), s.c. q.d.×14, Group 2 was treated with anti-PD-1 10 mg/kg i.p. q4d×4, Group 3 was treated with Compound 1 3 mg/kg s.c. q.d.×5D followed by 1 mg/kg s.c. q.d.×9D, Group 4 was treated with Compound 13 mg/kg s.c. q.d.×5D followed by 1 mg/kg s.c. q.d.×9D+anti-PD-1 10 mg/kg i.p. q4d×4, Group 5 was treated with Compound 11 mg/kg s.c. q.d.×14D and Group 6 was treated with Compound 11 mg/kg s.c. q.d.×14D+anti-PD-1 10 mg/kg i.p. q4d×4. The tumor sizes were measured three times per week during the treatment. The entire study was terminated on D42 after start of the treatment. In this study anti mPD-1 (catalog number BE0146) was used.

### II. Experimental Methods and Procedures

Animal Species: Mus musculus; Strain: BALB/c; Age: 6-8 weeks; Sex: female; Body weight (at treatment start): 20-24 g.

The 4T1 tumor cells were maintained in vitro as a monolayer culture in RPMI-1640 medium supplemented with 10% heat inactivated fetal bovine serum and 100 μg/mL penicillin streptomycin at 37° C. in an atmosphere of 5% CO₂ in air. The tumor cells were routinely subcultured twice weekly by trypsin-EDTA treatment. The cells growing to a confluency around 70%-80% were harvested and counted for tumor inoculation. The cultured 4T1 cells were harvested, re-suspended in base medium at a density of 2×10⁶ cells/mL with viability >90%. Each mouse was inoculated subcutaneously at the right flank with 1×10⁵ in 0.05 ml base medium for tumor development.

The treatments were started on day 6 after tumor inoculation when the tumor size reached 49-88 mm³ (average tumor size 59 mm³). Each group consisted of 8 tumor bearing mice. The testing article was administrated at the dosing volume indicated to the mice according to the predetermined regimen as shown Table 2A-1. Testing article formulations were prepared according to standard procedures. Details regarding tumor measurements and endpoints, and statistical analysis are provided in EXAMPLE 3.

TABLE 2A-1
Groups and Treatments for Efficacy Study

| | | Dose | |
|---|---|---|---|
| Group | Treatment | (mg/kg) | Dosing Route & Schedule |
| 1 | Vehicle control | 10 mL/kg | qdx14, s.c. |
| 2 | anti-PD-1 | 10 | q4dx4, i.p. |
| 3 | Cpd. 1 | 3/1 | 3 mpk qdx5, sc followed by |
| | | | 1 mpk, qdx9, s.c. |
| 4 | Cpd. 1 + | 3/1 + | 3 mpk qdx5, s.c. followed by |
| | aPD-1 | 10 | 1 mpk, qdx9, s.c.; q4dx4, i.p. |
| 5 | Cpd. 1 | 1 | qdx14, s.c. |
| 6 | Cpd. 1 + | 1 + | qdx14, s.c.; |
| | aPD-1 | 10 | q4dx4, i.p. |

### III. Results

### Body Weight Change

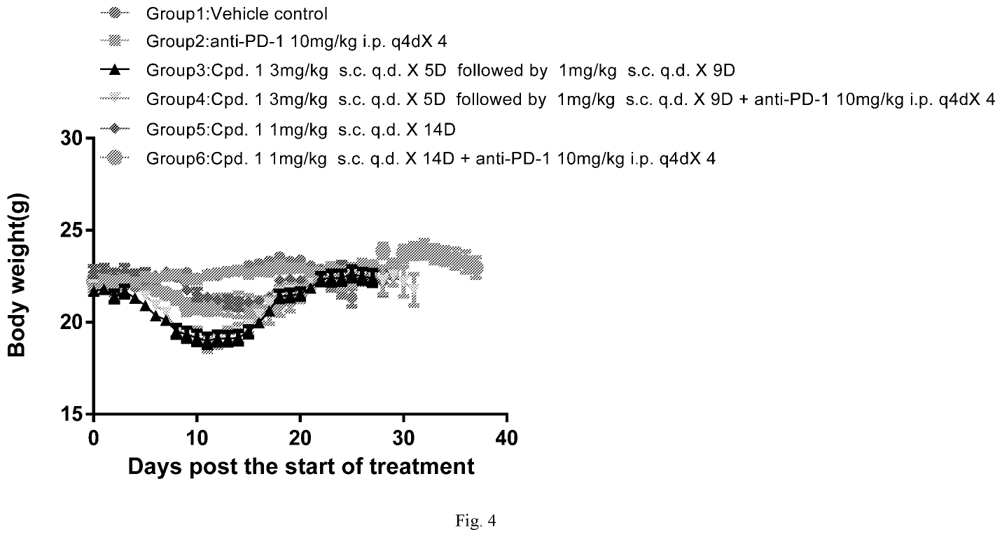

Body weight change in female C57BL/6 mice bearing 4T1 tumors are shown in FIG. 4. Once body weight loss exceeded 15%, the treatment was suspended. After treatment suspension, the body weight recovered quickly.

### Tumor Growth Analysis

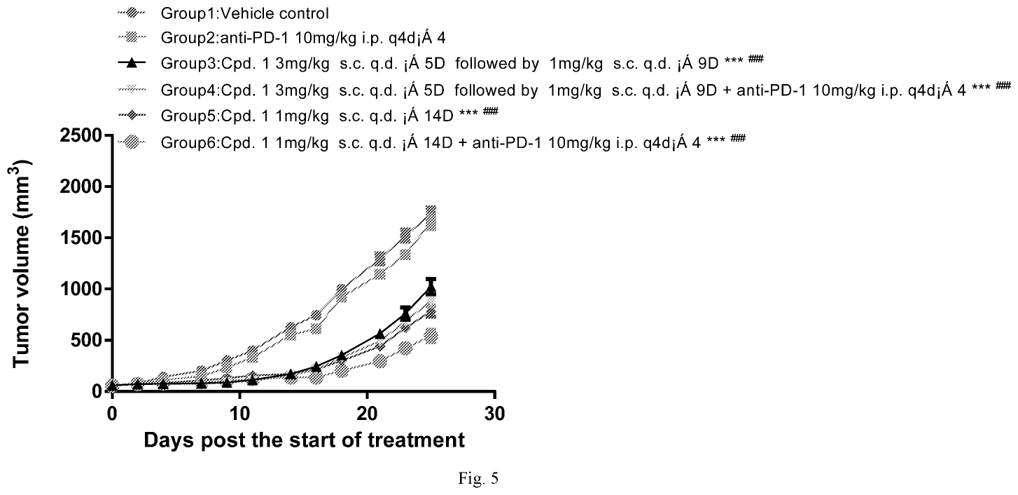

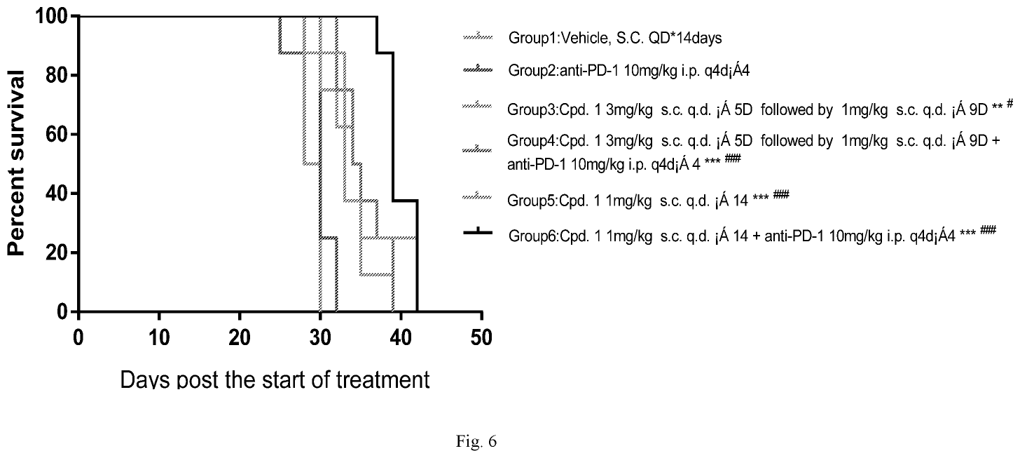

Mean tumor volume over time in female BALB/c mice bearing 4T1 tumors dosed with Compound 1 and Anti-PD-1 is shown in Table 2A-2 and FIG. 5. The tumor growth inhibition analysis is shown in Table 2A-3. The Kaplan-Meier survival curve (endpoint is defined as tumor volume reaching 2000 mm³) is shown in FIG. 6. The survival analysis is shown in Table 2A-4.

Group 3 and Group 4 induced some body weight loss but the other treatments were well-tolerated without any adverse effects observed in the 4T1 tumor bearing BALB/c mice. Compared to the vehicle control group, all treatments except anti-PD-1 10 mg/kg i.p. q4d×4 group showed significant inhibition on D25. Time-to-endpoint Kaplan-Meier survival analyses showed that all treatments except anti-PD-1 10 mg/kg i.p. q4d×4 group showed significant and unexpected survival benefits when compared to the vehicle group.

TABLE 2A-2
Tumor Volume over Time

| | Tumor volume (mm³)^{a} | | | | | |
|---|---|---|---|---|---|---|
| | | | | Cpd. 1 + | | Cpd. 1 + |
| | | | | Anti-PD-1 | | Anti-PD-1 |
| | | Anti-PD-1 | Cpd. 1 | 3/1 mg/kg + | Cpd. 1 | 1 mg/kg + |
| Days | Vehicle | 10 mg/kg | 3/1 mg/kg | 10 mg/kg | 1 mg/kg | 10 mg/kg |
| 0 | 61 ± 5 | 60 ± 4 | 59 ± 3 | 58 ± 3 | 58 ± 3 | 58 ± 3 |
| 2 | 80 ± 6 | 77 ± 4 | 72 ± 4 | 71 ± 2 | 75 ± 4 | 73 ± 4 |
| 4 | 142 ± 11 | 112 ± 5 | 76 ± 3 | 74 ± 2 | 85 ± 4 | 79 ± 3 |
| 7 | 204 ± 12 | 152 ± 9 | 80 ± 3 | 79 ± 2 | 108 ± 4 | 92 ± 6 |
| 9 | 302 ± 16 | 234 ±13 | 88 ± 6 | 87 ± 5 | 130 ± 10 | 102 ± 7 |
| 11 | 399 ± 16 | 332 ± 14 | 113 ± 9 | 106 ± 7 | 158 ± 12 | 122 ± 10 |
| 14 | 628 ± 29 | 550 ± 27 | 171 ± 12 | 158 ± 17 | 172 ± 11 | 138 ± 10 |
| 16 | 745 ± 25 | 613 ± 24 | 245 ± 17 | 214 ± 23 | 215 ± 17 | 138 ± 14 |
| 18 | 999 ± 33 | 921 ± 36 | 356 ± 27 | 333 ± 30 | 300 ± 23 | 203 ± 27 |
| 21 | 1296 ± 54 | 1147 ± 48 | 565 ± 37 | 493 ± 28 | 441 ± 31 | 296 ± 33 |
| 23 | 1521 ± 59 | 1337 ± 41 | 762 ± 62 | 684 ± 48 | 622 ± 44 | 421 ± 45 |
| 25 | 1735 ± 64 | 1654 ± 69 | 1024 ± 75 | 884 ± 55 | 782 ± 53 | 545 ± 58 |

^{a}Mean ± SEM;
n = 8

TABLE 2A-3
Tumor Growth Inhibition Calculation Based on TV Measurements at Day 25

| | Tumor Size | | | | | | |
|---|---|---|---|---|---|---|---|
| | (mm³)^{a} | RTV | T/C | TGI | p | p | p |
| Treatment | at day 25 | at day 25 | (%) | (%) | value^{b} | value^{c} | value^{d} |
| Vehicle control | 1735 ± 64 | 29.61 ± 2.22 | — | — | — | | |
| Anti-PD-1 (10 mg/kg) | 1654 ± 69 | 28.55 ± 2.02 | 96.42 | 3.58 | ns | | |
| Cpd. 1 (3/1 mg/kg) | 1024 ± 75 | 17.45 ± 0.83 | 58.95 | 41.05 | <0.001 | | |
| Cpd. 1 (3/1 mg/kg) + | 884 ± 55 | 15.59 ± 1.47 | 52.67 | 47.33 | <0.001 | <0.001 | <0.01 |
| Anti-PD-1 (10 mg/kg) | | | | | | | |
| Cpd. 1 (1 mg/kg) | 782 ± 53 | 13.65 ± 1.13 | 46.10 | 53.9 | <0.001 | | |
| Cpd. 1 (1 mg/kg) + | 545 ± 58 | 9.4 ± 0.99 | 31.76 | 68.24 | <0.001 | <0.001 | <0.001 |
| Anti-PD-1 (10 mg/kg) | | | | | | | |

^{a}Mean ± SEM;
^{b}All groups compare to G1;
^{c}Combination group compare to G2;
^{d}Combination group compare to Compound 1 monotherapy group

TABLE 2A-4
Survival Analysis

| | MST | ILS | p | p | p |
|---|---|---|---|---|---|
| Treatment | (day) | (%) | value^{a} | value^{b} | value^{c} |
| Vehicle control | 29.0 | — | — | | |
| Anti-PD-1 (10 mg/kg) | 30.0 | 3.4 | ns | | |
| Compound 1 (3/1 mg/kg) | 34.5 | 19.0 | <0.01 | | |
| Compound 1 (3/1 mg/kg) + Anti-PD-1 (10 mg/kg) | 34.5 | 19.0 | <0.001 | <0.001 | ns |
| Compound 1 (1 mg/kg) | 33.0 | 13.8 | <0.001 | | |
| Compound 1 (1 mg/kg) + Anti-PD-1 (10 mg/kg) | 39.0 | 34.5 | <0.001 | <0.001 | ns |

### Example 2B

### In Vivo Test of Anti-Tumor Efficacy of Compound 1 with and without Anti-PD-1 in the CT26.WT Murine Colorectal Model in Female BALB/c Mice

### I. Abstract

Female BALB/c mice were inoculated subcutaneously at right flank with CT26.WT cells for tumor development. Five days after tumor inoculation, 48 mice with tumor size ranging from 39-61 mm³ (average tumor size 49 mm³) were selected and assigned into 6 groups using stratified randomization with 8 mice per group based upon their tumor volumes. The treatments were started from the day of randomization (defined as D0), Group 1 was treated with Vehicle (Tween 80:Ethanol:Saline—5:5:90 v/v/v), s.c. q.d.×14, Group 2 was treated with anti-PD-1 10 mg/kg i.p. q4d×4, Group 3 was treated with Compound 1 3 mg/kg s.c. q.d.×5D followed by 1 mg/kg s.c. q.d.×9D, Group 4 was treated with Compound 1 3 mg/kg s.c. q.d.×5D followed by 1 mg/kg s.c. q.d.×9D+anti-PD-1 10 mg/kg i.p. q4d×4, Group 5 was treated with Compound 1 1 mg/kg s.c. q.d.×14D and Group 6 was treated with Compound 1 1 mg/kg s.c. q.d.×14D+anti-PD-1 10 mg/kg i.p. q4d×4. The tumor sizes were measured three times per week during the treatment. The entire study was terminated on D64 after start of the treatment.

### II. Experimental Methods and Procedures

Animal Species: Mus musculus; Strain: BALB/c; Age: 6-8 weeks; Sex: female; Body weight (at treatment start): 20-24 g.

The CT26.WT tumor cells (ATCC, Cat #CRL-2638™) were maintained in vitro as a monolayer culture in RPMI 1640 medium supplemented with 10% heat inactivated fetal bovine serum, 100 U/mL penicillin and 100 μg/mL streptomycin, and L-glutamine (2 mM) at 37° C. in an atmosphere of 5% CO2 in air. The tumor cells were routinely subcultured twice weekly by trypsin-EDTA treatment. The cells growing to a confluency around 70%-80% were harvested and counted for tumor inoculation. The cultured CT26.WT cells were harvested, re-suspended in base medium with viability>90%. Each mouse was inoculated subcutaneously at the right flank with 3×10⁵ in 0.05 ml base medium for tumor development.

The treatments were started on day 5 after tumor inoculation when the tumor size reached 39-61 mm³ (average tumor size 49 mm³). Each group consisted of 8 tumor bearing mice. The testing article was administrated at the dosing volume indicated to the mice according to the predetermined regimen as shown Table 2B-1. Testing article formulations were prepared according to standard procedures. Details regarding tumor measurements and endpoints, and statistical analysis are provided in EXAMPLE 3.

TABLE 2B-1
Groups and Treatments for Efficacy Study

| | | Dose | |
|---|---|---|---|
| Group | Treatment | (mg/kg) | Dosing Route & Schedule |
| 1 | Vehicle | 10 mL/kg | qdx14, s.c. |
| | control | | |
| 2 | anti-PD-1 | 10 | q4dx4, i.p. |
| 3 | Cpd. 1 | 3/1 | 3 mpk qdx5, sc followed by 1 |
| | | | mpk, qdx9, s.c. |
| 4 | Cpd. 1 + | 3/1 + | 3 mpk qdx5, s.c. followed by 1 |
| | aPD-1 | 10 | mpk, qdx9, s.c.; q4dx4, i.p. |
| 5 | Cpd. 1 | 1 | qdx14, s.c. |
| 6 | Cpd. 1 + | 1 + | qdx14, s.c.; |
| | aPD-1 | 10 | q4dx4, i.p. |

### III. Results

### Body Weight Change

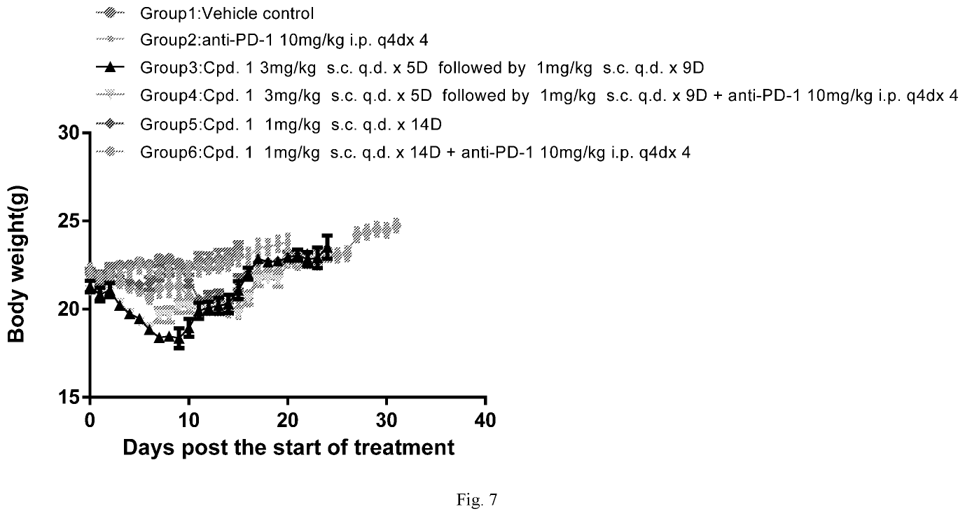

Body weight change in female C57BL/6 mice bearing CT26.WT tumors are shown in FIG. 7. Once body weight loss was exceeding 15%, the treatment was suspended. After treatment suspension, the body weight recovered quickly.

### Tumor Growth Analysis

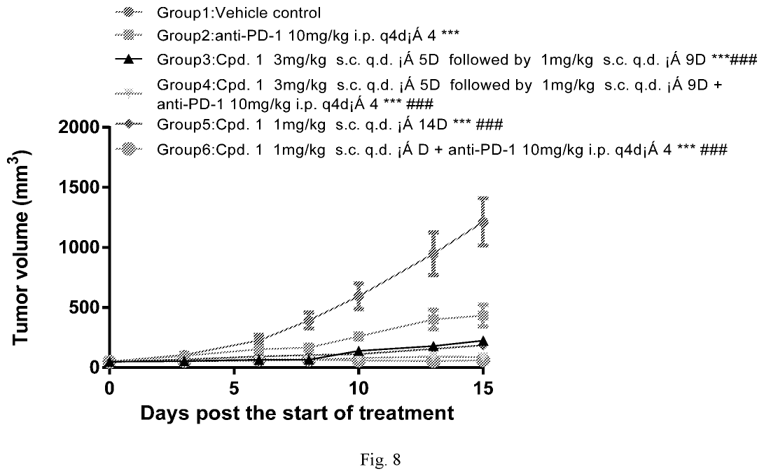

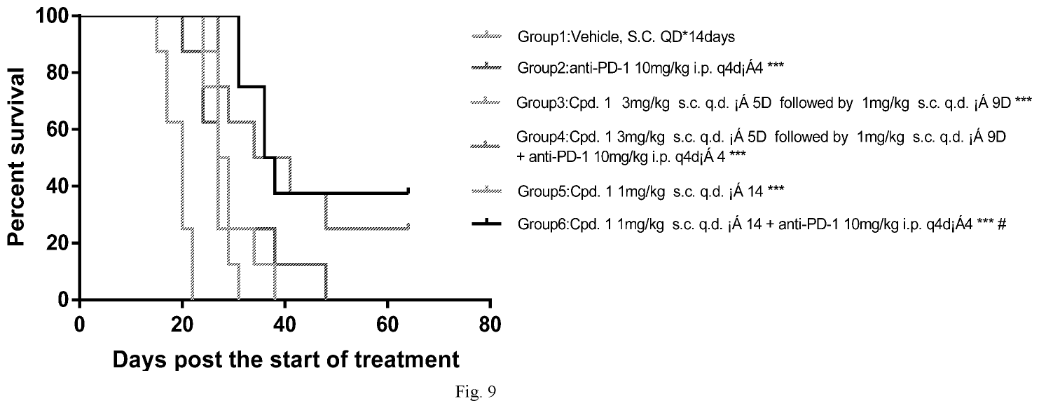

Mean tumor volume over time in female BALB/c mice bearing CT26.WT tumors dosed with Compound 1 and Anti-PD-1 is shown in Table 2B-2 and FIG. 8. The tumor growth inhibition analysis is shown in Table 2B-3. The Kaplan-Meier survival curve (endpoint is defined as tumor volume reaching 2000 mm3) is shown in FIG. 9. The survival analysis is shown in Table 2B-4.

Group 3 induced some body weight loss but the other treatments were well-tolerated without any adverse effects observed in the CT26.WT tumor bearing BALB/c mice. Compared to the vehicle control group, all treatments showed significant inhibition on D15. Time-to-endpoint Kaplan-Meier survival analyses showed that all treatments showed significant survival benefits when compared to the vehicle group. Groups 4 and 6 also demonstrated significant improvement in tumor growth inhibition and survival when compared to monotherapy control groups.

TABLE 2B-2
Tumor Volume over Time

| | Tumor volume (mm³)^{a} | | | | | |
|---|---|---|---|---|---|---|
| | | | | Cpd. 1 + | | Cpd. 1 + |
| | | | | Anti-PD-1 | | Anti-PD-1 |
| | | Anti-PD-1 | Cpd. 1 | 3/1 mg/kg + | Cpd. 1 | 1 mg/kg + |
| Days | Vehicle | 10 mg/kg | 3/1 mg/kg | 10 mg/kg | 1 mg/kg | 10 mg/kg |
| 0 | 49 ± 2 | 49 ± 2 | 49 ± 2 | 49 ± 2 | 49 ± 2 | 49 ± 2 |
| 3 | 106 ± 13 | 97 ± 5 | 54 ± 2 | 52 ± 3 | 68 ± 6 | 56 ± 3 |
| 6 | 226 ± 45 | 152 ± 14 | 65 ± 3 | 59 ± 4 | 92 ± 8 | 68 ± 5 |
| 8 | 392 ± 67 | 165 ± 21 | 139 ± 14 | 63 ± 7 | 104 ± 9 | 66 ± 9 |
| 10 | 595 ± 107 | 259 ± 37 | 180 ± 20 | 80 ± 12 | 112 ± 9 | 59 ± 9 |
| 13 | 947 ± 178 | 401 ± 82 | 224 ± 25 | 90 ± 19 | 156 ± 18 | 54 ± 9 |
| 15 | 1212 ± 197 | 432 ± 91 | 333 ± 41 | 87 ± 18 | 186 ± 21 | 60 ± 13 |

^{a}Mean ± SEM; n = 8

TABLE 2B-3
Tumor Growth Inhibition Calculation Based on TV Measurements at Day 55

| | Tumor | | | | | | |
|---|---|---|---|---|---|---|---|
| | Size | | | | | | |
| | (mm³)^{a} | RTV | T/C | TGI | p | p | p |
| Treatment | at day 15 | at day 15 | (%) | (%) | value^{b} | value^{c} | value^{d} |
| Vehicle control | 1212 ± 197 | 25.24 ± 4.26 | — | — | — | | |
| Anti-PD-1 (10 mg/kg) | 432 ± 91 | 9.10 ± 2.05 | 36.06 | 67.06 | <0.001 | | |
| Cpd. 1 (3/1 mg/kg) | 224 ± 25 | 4.79 ± 0.69 | 19.00 | 84.90 | <0.001 | | |
| Cpd. 1 (3/1 mg/kg) + | 87 ± 18 | 1.81 ± 0.41 | 7.15 | 96.72 | <0.001 | <0.001 | <0.001 |
| Anti-PD-1 (10 mg/kg) | | | | | | | |
| Cpd. 1 (1 mg/kg) | 186 ± 21 | 3.84 ± 0.43 | 15.23 | 188.18 | <0.001 | | |
| Cpd. 1 (1 mg/kg) + | 60 ± 13 | 1.24 ± 0.24 | 4.92 | 99.01 | <0.001 | <0.001 | <0.001 |
| Anti-PD-1 (10 mg/kg) | | | | | | | |

^{a}Mean ± SEM;
^{b}All groups compare to G1;
^{c}Combination group compare to G2;
^{d}Combination group compare to Compound 1 monotherapy group

TABLE 2B-4
Survival Analysis

| | MST | ILS | p | p | p |
|---|---|---|---|---|---|
| Treatment | (day) | (%) | value^{a} | value^{b} | value^{c} |
| Vehicle control | 20 | — | — | | |
| Anti-PD-1 (10 mg/kg) | 28 | 40 | <0.001 | | |
| Compound 1 (3/1 mg/kg) | 27 | 35 | <0.001 | | |
| Compound 1 (3/1 mg/kg) + Anti-PD-1 (10 mg/kg) | 38 | 90 | <0.001 | ns | <0.01 |
| Compound 1 (1 mg/kg) | 28 | 40 | <0.001 | | |
| Compound 1 (1 mg/kg) + Anti-PD-1 (10 mg/kg) | 37 | 85 | <0.001 | <0.05 | <0.01 |

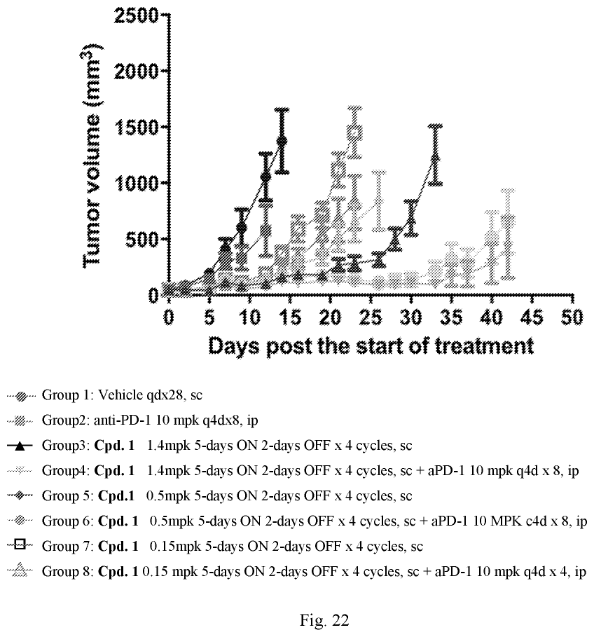

In a similar experiment using the CT26 model, the combination of Compound 1 and anti-PD-1 treatment was tested using a 5-day ON 2-day OFF dosing schedule for Compound 1. See Table 2B-6. The combination of Compound 1 and anti-PD-1 treatment demonstrated a significant and unexpected increase in survival at 1.4, 0.5, and 0.15 mg/kg doses of Compound 1 as shown in Table 2B-5. Significant antitumor activity and survival benefit was observed compared to either anti-PD-1 or Compound 1 alone (p<0.001) in all dose groups as shown in FIG. 22. This synergistic effect led to a surprising number of dose-dependent long-term durable cures in the combination groups with 12.5, 37.5 and 62.5% cures in the 0.15, 0.5, and 1.4 mg/kg Compound 1 combination groups, respectively.

TABLE 2B-5

| | Dose | Anti- | Median | | |
|---|---|---|---|---|---|
| | Compound 1 | PD-1 | Survival | | |
| Group | (mg/kg) | (mg/kg) | (Days) | pValue | Cures (%) |
| Vehicle | — | — | 17.5 | — | 0 |
| Anti-PD-1 | — | 10 | 31.5 | p < 0.01 | 0 |
| Compound 1 | 1.4 | — | 37 | p < 0.001 | 0 |
| Compound 1 + | 1.4 | 10 | >77 | p < 0.001 | 62.5 |
| Anti-PD1 | | | | | |
| Compound 1 | 0.5 | — | 36 | p < 0.001 | 0 |
| Compound 1 + | 0.5 | 10 | 56 | p < 0.001 | 37.5 |
| Anti-PD1 | | | | | |
| Compound 1 | 0.15 | — | 26 | p < 0.001 | 0 |
| Compound 1 + | 0.15 | 10 | 30 | p < 0.001 | 12.5 |
| Anti-PD1 | | | | | |

TABLE 2B-6

| | | | | Mean | | | | |
|---|---|---|---|---|---|---|---|---|
| | | | | Tumor | | p-Value | p-Value | p-Value |
| Test | Dose | Route and | BWC | Vol | TGI | vs | vs anti- | vs |
| Article | (mg/kg) | Schedule | (%)^{a} | (mm³)^{b} | (%)^{b} | Vehicle | PD-1 | Cpd. 1 |
| Vehicle | — | | 10 | 1054 ± 209 | NA | NA | NA | NA |
| Anti- | 10 | Q4Dx8 | 7 | 574 ± 222 | 48 | <0.05 | NA | NA |
| PD-1 | | | | | | | | |
| Cpd. 1 | 1.4 | 5-day ON 2- | −11 | 106 ± 16 | 91 | <0.001 | NA | NA |
| | | day OFFx 4 | | | | | | |
| | | cycles | | | | | | |
| Cpd. 1 + | 1.4 + 10 | 5-day ON 2- | −8 | 72 ± 7 | 95 | <0.001 | p < 0.001 | p < 0.001 |
| anti-PD- | | day OFFx 4 | | | | | | |
| 1 | | cycles + Q4D | | | | | | |
| | | x 8 | | | | | | |
| Cpd. 1 | 0.5 | 5-day ON 2- | 1.4 | 150 ± 21 | 88 | <0.001 | NA | NA |
| | | day OFFx 4 | | | | | | |
| | | cycles | | | | | | |
| Cpd. 1 + | 0.5 + 10 | 5-day ON 2- | 2 | 112 ± 10 | 91 | <0.001 | p < 0.001 | p < 0.001 |
| anti-PD- | | day OFFx 4 | | | | | | |
| 1 | | cycles + Q4D | | | | | | |
| | | x 8 | | | | | | |
| Cpd. 1 | 0.15 | 5-day ON 2- | 7 | 188 ± 29 | 76 | <0.001 | NA | NA |
| | | day OFFx 4 | | | | | | |
| | | cycles | | | | | | |
| Cpd. 1 + | 0.15 + 10 | 5-day ON 2- | 6 | 138 ± 28 | 87 | <0.001 | p < 0.001 | p < 0.001 |
| anti-PD- | | day OFFx4 | | | | | | |
| 1 | | cycles + Q4D | | | | | | |
| | | x 8 | | | | | | |

^{a}Determined on Day 12;
^{b}Determined on Day 15;
NA: not applicable

In a follow-up study, the nine mice that were “cured” by combination treatment (5 mice from the 1.4 mg/kg group, 3 mice from the 0.5 mg/kg group, and 1 mouse from the 0.15 mg/kg group) and eight naïve mice were rechallenged with CT26 tumors. All nine of the cured mice rejected re-implantation of CT26 tumors. None of the naïve mice rejected re-implantation of CT26 tumors. These data suggest that the combination of Compound 1 and anti-PD-1 invokes an unexpected long-term immune memory response.

### Example 2C

### In Vivo Test of Anti-Tumor Efficacy of Compound 1 with and without Anti-PD-1 in the Subcutaneous EL4 Murine Lymphoma Model in Female C57BL/6

### I. Abstract

Female C57BL/6 mice were inoculated subcutaneously at the right flank with EL4 cells for tumor development. Five days after tumor inoculation, 48 mice with tumor size ranging from 50-76 mm³ (average tumor size 65 mm³) were selected and assigned into 6 groups using stratified randomization with 8 mice per group based upon their tumor volumes. The treatments were started from the day of randomization (defined as D0), Group 1 was treated with Vehicle (Tween 80:Ethanol:Saline—5:5:90 v/v/v), s.c., qd×4, Group 2 was treated with anti-PD-1 10 mg/kg i.p. q4d×4, Group 3 was treated with Compound 1 0.3 mg/kg s.c. q.d.×14, Group 4 was treated with Compound 1 0.3 mg/kg s.c. q.d.×14+anti-PD-1 10 mg/kg i.p. q4d×4, Group 5 was treated with Compound 1 1 mg/kg s.c. q.d.×14, Group 6 was treated with Compound 1 1 mg/kg s.c. q.d.×14+anti-PD-1 10 mg/kg i.p. q4d×4. The tumor sizes were measured three times per week during the treatment. The entire study was terminated on D19 after start of the treatment.

### II. Experimental Methods and Procedures

Animal Species: Mus musculus; Strain: BALB/c; Age: 6-8 weeks; Sex: female; Body weight (at treatment start): 17-22 g.

The EL4 tumor cells were maintained in vitro as a monolayer culture in DMEM medium supplemented with 10% heat inactivated horse serum and 100 μg/mL penicillin streptomycin at 37° C. in an atmosphere of 5% CO2 in air. The tumor cells were routinely subcultured twice weekly by trypsin-EDTA treatment. The cells growing to a confluency around 70%-80% were harvested and counted for tumor inoculation. The cultured EL4 were harvested, re-suspended in base medium at a density of 8.79×10⁵ cells/mL with viability>90%. Each mouse was inoculated subcutaneously at the right flank with 2×10⁵ in 0.1 ml base medium for tumor development.

The treatments were started on day 5 after tumor inoculation when the tumor size reached 50-76 mm³ (average tumor size 65 mm³). Each group consisted of 8 tumor bearing mice. The testing article was administrated at the dosing volume indicated to the mice according to the predetermined regimen as shown Table 2C-1. Testing article formulations were prepared according to standard procedures. Details regarding tumor measurements and endpoints, and statistical analysis are provided in EXAMPLE 3.

TABLE 2C-1
Groups and Treatments for Efficacy Study

| | | Dose | |
|---|---|---|---|
| Group | Treatment | (mg/kg) | Dosing Route & Schedule |
| 1 | Vehicle control | 10 mL/kg | qd x 14, s.c. |
| 2 | anti-PD-1 | 10 | q4d x 4, ip |
| 3 | Cpd. 1 | 0.3 | qd x 14, s.c. |
| 4 | Cpd. 1 + | 0.3 + 10 | qd x 14, s.c.; + |
| | anti-PD-1 | | q4d x 4, ip |
| 5 | Cpd. 1 | 1 | qd x 14, sc |
| 6 | Cpd. 1 + | 1 + 10 | qd x 14, s.c.; + |
| | anti-PD-1 | | q4d x 4 days, ip |

### III. Results

### Body Weight Change

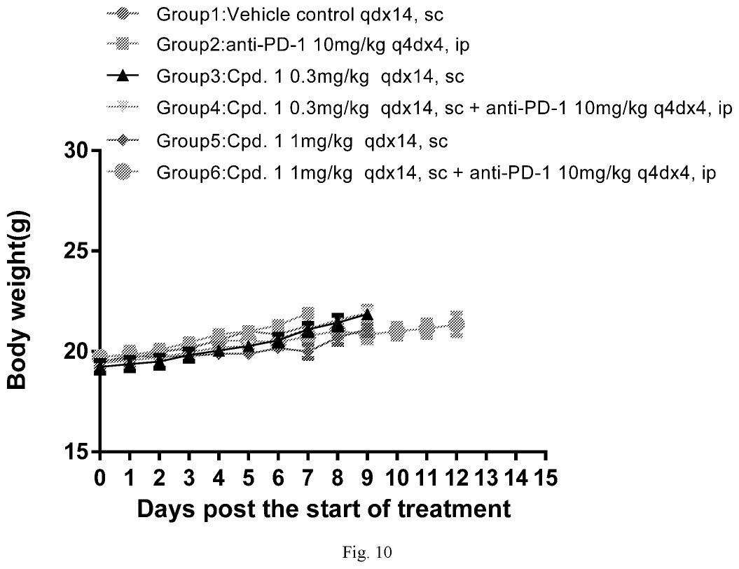

Body weight change in female C57BL/6 mice bearing EL4 tumors are shown in FIG. 10.

### Tumor Growth Analysis

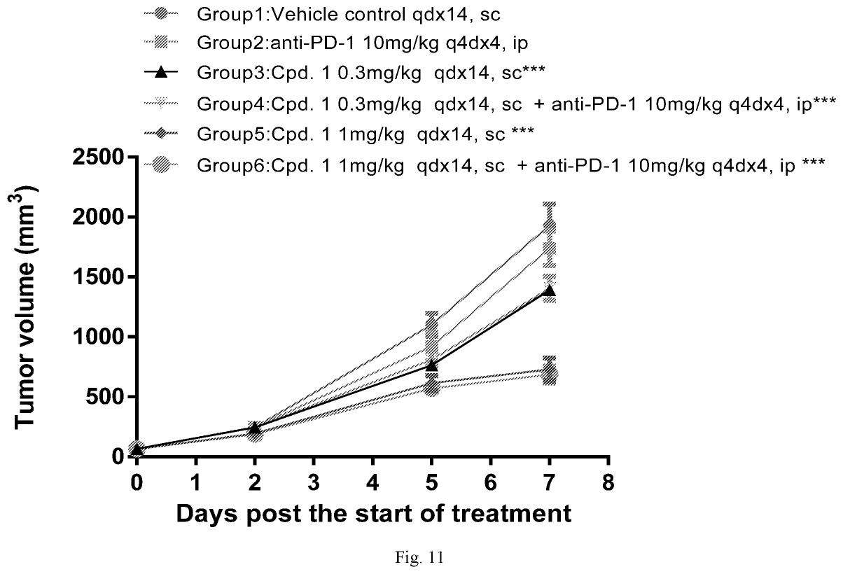

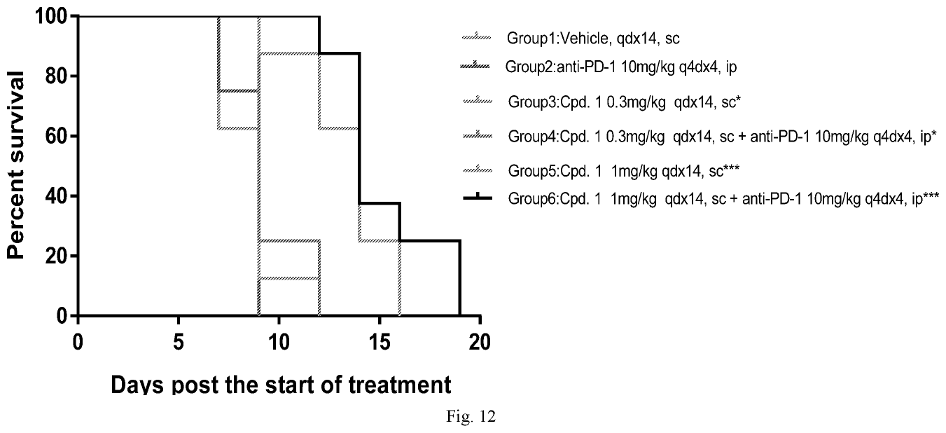

Mean tumor volume over time in female C57BL/6 mice bearing EL4 tumors dosed with Compound 1 and anti-PD-1 is shown in Table 2C-2 and FIG. 11. The tumor growth inhibition analysis is shown in Table 2C-3. The Kaplan-Meier survival curve (endpoint is defined as tumor volume reaching 2000 mm³) is shown in FIG. 12. The survival analysis is shown in Table 2C-4.

All treatments were well-tolerated without any adverse effect observed by the EL4 tumor bearing C57BL/6 mice. Compared to vehicle control group, all treatments except anti-PD-1 monotherapy group showed significant inhibition on D7. Time-to-endpoint Kaplan-Meier survival analyses showed Groups 5 and 6 showed significant and unexpected survival benefit when compared to the vehicle group.

TABLE 2C-2
Tumor Volume over Time

| | Tumor volume (mm³)^{a} | | | | | |
|---|---|---|---|---|---|---|
| | | | | Cpd. 1 + | | Cpd. 1 + |
| | | | | Anti-PD-1 | | Anti-PD-1 |
| | | Anti-PD-1 | Cpd. 1 | 0.3 mg/kg + | Cpd. 1 | 1 mg/kg + |
| Days | Vehicle | 10 mg/kg | 0.3 mg/kg | 10 mg/kg | 1 mg/kg | 10 mg/kg |
| 0 | 65 ± 3 | 65 ± 2 | 65 ± 3 | 65 ± 2 | 65 ± 3 | 65 ± 3 |
| 2 | 246 ± 36 | 246 ± 31 | 245 ± 6 | 246 ± 18 | 194 ± 15 | 189 ± 21 |
| 5 | 1101 ± 96 | 920 ± 118 | 763 ± 29 | 810 ± 56 | 613 ± 62 | 570 ± 45 |
| 7 | 1929 ± 177 | 1739 ± 144 | 1391 ± 41 | 1405 ± 100 | 728 ± 96 | 687 ± 70 |

^{a}Mean ± SEM;
n = 8

TABLE 2C-3
Tumor Growth Inhibition Calculation Based on TV Measurements at Day 7

| | Tumor Size | | | | |
|---|---|---|---|---|---|
| | (mm³)^{a} | RTV | T/C | | |
| Treatment | at day 7 | at day 7 | (%) | TGI (%) | p value^{b} |
| Vehicle control | 1929 ± 177 | 29.66 ± 2.40 | — | — | — |
| Anti-PD-1 (10 mg/kg) | 1739 ± 144 | 26.85 ± 2.23 | 90.55 | 10.21 | ns |
| Cpd. 1 (0.3 mg/kg) | 1391 ± 41 | 21.9 ± 1.51 | 73.86 | 28.88 | <0.0001 |
| Cpd. 1 (0.3 mg/kg) + | 1405 ± 100 | 21.65 ± 1.47 | 73.02 | 28.13 | <0.0001 |
| Anti-PD-1 (10 mg/kg) | | | | | |
| Cpd. 1 (1 mg/kg) | 728 ± 96 | 11.2 ± 1.25 | 37.76 | 64.42 | <0.0001 |
| Cpd. 1 (1 mg/kg) + | 687 ± 70 | 10.65 ± 1.18 | 35.91 | 66.67 | <0.0001 |
| Anti-PD-1 (10 mg/kg) | | | | | |

^{a}Mean ± SEM;
^{b}p value calculated based on tumor size

TABLE 2C-4
Survival Analysis

| | MST | ILS | |
|---|---|---|---|
| Treatment | (day) | (%) | p value^{a} |
| Vehicle control | 9 | — | — |
| Anti-PD-1 (10 mg/kg) | 9 | 0 | >0.05 |
| Compound 1 (0.3 mg/kg) | 9 | 0 | <0.05 |
| Compound 1 (0.3 mg/kg) ± | 9 | 0 | <0.05 |
| Anti-PD-1 (10 mg/kg) | | | |
| Compound 1 (1 mg/kg) | 14 | 35.7 | <0.001 |
| Compound 1 (1 mg/kg) ± | 14 | 35.7 | <0.001 |
| Anti-PD-1 (10 mg/kg) | | | |

### Example 2D

### In Vivo Test of Anti-Tumor Efficacy of Compound 1 with and without Anti-PD-1 in the Subcutaneous MC-38 Murine Colorectal Cancer Model in Female

### I. Abstract

Female C57BL/6 mice were inoculated subcutaneously at right flank with MC-38 cells for tumor development. Five days after tumor inoculation, 48 mice with tumor size ranging from 50-100 mm³ (average tumor size 72 mm³) were selected and assigned into six groups using stratified randomization with 8 mice per group based upon their tumor volumes. The treatments were started from the day of randomization (defined as D0), Group 1 was treated with Vehicle (Tween 80:Ethanol:Saline—5:5:90 v/v/v), s.c., qd×14, Group 2 was treated with Anti-PD-1 10 mg/kg i.p. q4d×4, Group 3 was treated with Compound 1 0.3 mg/kg s.c. q.d.×14, Group 4 was treated with Compound 1 0.3 mg/kg s.c. q.d.×14+Anti-PD-1 10 mg/kg i.p. q4d×4, Group 5 was treated with Compound 1 1 mg/kg s.c. q.d.×14, Group 6 was treated with Compound 1 1 mg/kg s.c. q.d.×14+Anti-PD-1 10 mg/kg i.p. q4d×4. The tumor sizes were measured three times per week during the treatment. The entire study was terminated on D37 after start of the treatment.

### II. Experimental Methods and Procedures

Animal Species: Mus musculus; Strain: BALB/c; Age: 6-8 weeks; Sex: female; Body weight (at treatment start): 18-22 g.

The MC-38 tumor cells were maintained in vitro as a monolayer culture in DMEM medium supplemented with 10% heat inactivated fetal bovine serum and 100 μg/mL penicillin streptomycin at 37° C. in an atmosphere of 5% CO₂ in air. The tumor cells were routinely subcultured twice weekly by trypsin-EDTA treatment. The cells growing to a confluency around 70%-80% were harvested and counted for tumor inoculation. The cultured MC-38 were harvested, re-suspended in base medium at a density of 1×10⁷ cells/mL with viability >90%. Each mouse was inoculated subcutaneously at the right flank with 1×10⁶ cells in 0.1 ml base medium for tumor development.

The treatments were started on day 5 after tumor inoculation when the tumor size reached 50-100 mm³ (average tumor size 72 mm³). Each group consisted of 8 tumor bearing mice.

The testing article was administrated at the dosing volume indicated to the mice according to the predetermined regimen as shown Table 2D-1. Testing article formulations were prepared according to standard procedures. Details regarding tumor measurements and endpoints, and statistical analysis are provided in EXAMPLE 3.

TABLE 2D-1
Groups and Treatments for Efficacy Study

| | | Dose | |
|---|---|---|---|
| Group | Treatment | (mg/kg) | Dosing Route & Schedule |
| 1 | Vehicle control | 10 mL/kg | qd x 14, s.c. |
| 2 | anti-PD-1 | 10 | q4d x 4, ip |
| 3 | Cpd. 1 | 0.3 | qd x 14, s.c. |
| 4 | Cpd. 1 + | 0.3 + 10 | qd x 14, s.c.; + |
| | anti-PD-1 | | q4d x 4, ip |
| 5 | Cpd. 1 | 1 | qd x 14, sc |
| 6 | Cpd. 1 + | 1 + 10 | qd x 14, s.c.; + |
| | anti-PD-1 | | q4d x 4 days, ip |

### III. Results

### Body Weight Change

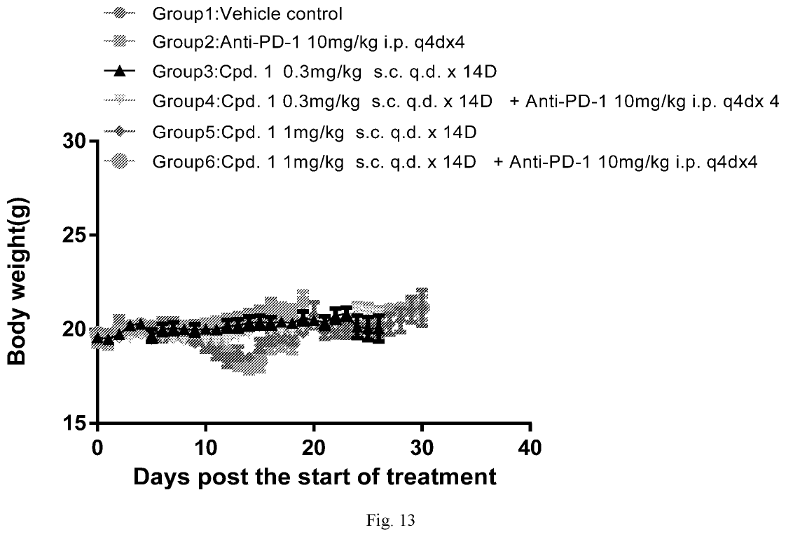

Body weight change in female C57BL/6 mice bearing MC-38 tumors are shown in FIG. 13.

### Tumor Growth Analysis

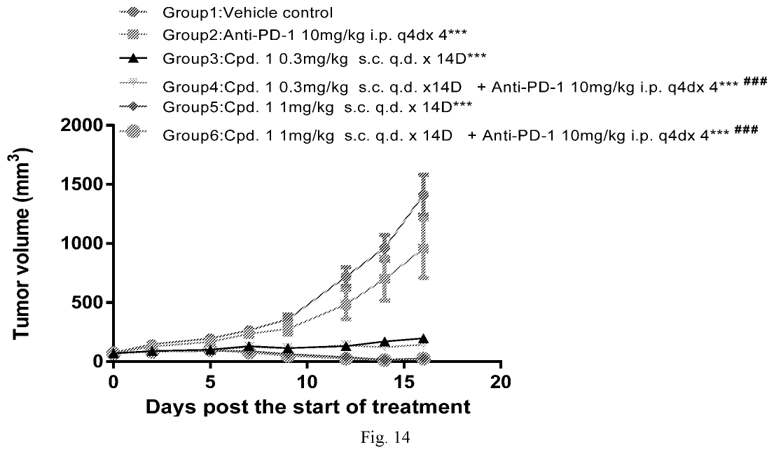

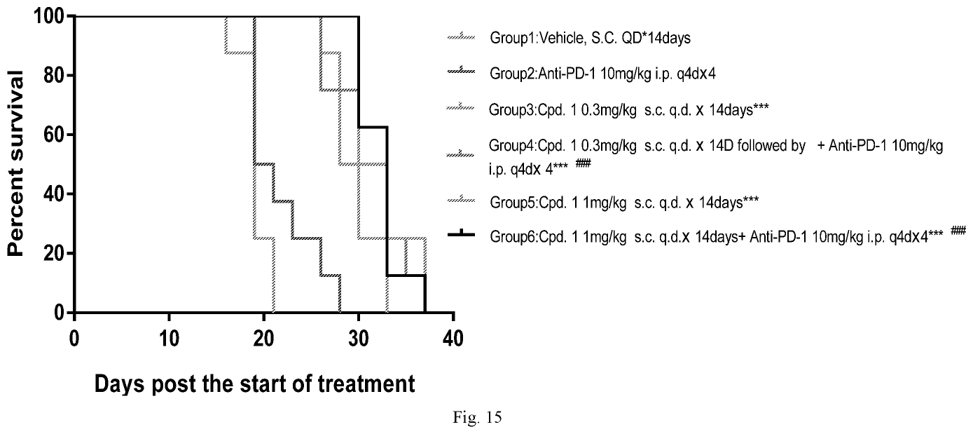

Mean tumor volume over time in female C57BL/6 mice bearing MC-38 tumors dosed with Anti-PD-1 and Compound 1 is shown in Table 2D-2 and FIG. 14. The tumor growth inhibition analysis is shown in Table 2D-3. The Kaplan-Meier survival curve (endpoint is defined as tumor volume reaching 2000 mm³) is shown in FIG. 15. The survival analysis is shown in Table 2D-4.

Group 3 and Group 6 induced some body weight loss but the other treatments were well-tolerated without any adverse effects observed in the MC-38 tumor bearing C57BL/6 mice. Compared to the vehicle control group, all treatments showed significant inhibition on D16. Time-to-endpoint Kaplan-Meier survival analyses showed that all treatment groups except Anti-PD-1 10 mg/kg monotherapy group showed significant and unexpected survival benefits when compared to the vehicle (G1) group

TABLE 2D-2
Tumor Volume over Time

| | Tumor volume (mm³)^{a} | | | | | |
|---|---|---|---|---|---|---|
| | | | | Cpd. 1 + | | Cpd. 1 + |
| | | | | Anti-PD-1 | | Anti-PD-1 |
| | | Anti-PD-1 | Cpd. 1 | 0.3 mg/kg + | Cpd. 1 | 1 mg/kg + |
| Days | Vehicle | 10 mg/kg | 0.3 mg/kg | 10 mg/kg | 1 mg/kg | 10 mg/kg |
| 0 | 73 ± 6 | 72 ± 6 | 71 ± 6 | 72 ± 6 | 71 ± 6 | 71 ± 6 |
| 2 | 147 ± 14 | 127 ± 10 | 88 ± 5 | 85 ± 8 | 86 ± 4 | 83 ± 7 |
| 5 | 196 ± 17 | 167 ± 15 | 101 ± 5 | 96 ± 9 | 91 ± 5 | 98 ± 8 |
| 7 | 266 ± 28 | 235 ± 31 | 131 ± 8 | 125 ± 13 | 89 ± 3 | 77 ± 6 |
| 9 | 359 ± 39 | 277 ± 49 | 144 ± 13 | 112 ± 13 | 65 ± 4 | 49 ± 5 |
| 12 | 719 ± 78 | 484 ± 124 | 131 ± 15 | 138 ± 22 | 38 ± 4 | 25 ± 6 |
| 14 | 962 ± 109 | 701 ± 185 | 171 ± 32 | 122 ± 23 | 15 ± 5 | 13 ± 4 |
| 16 | 1412 ± 170 | 957 ± 250 | 198 ± 36 | 144 ± 30 | 28 ± 7 | 27 ± 6 |

^{a}Mean ± SEM;
n = 8

TABLE 2D-3
Tumor Growth Inhibition Calculation Based on TV Measurements at Day 7

| | Tumor Size | | | | | | |
|---|---|---|---|---|---|---|---|
| | (mm³)^{a} | RTV | T/C | TGI | p | p | p |
| Treatment | at day 16 | at day 16 | (%) | (%) | value^{b} | value^{c} | value^{d} |
| Vehicle control | 1412 ± 170 | 19.41 ± 1.77 | — | — | — | | |
| Anti-PD-1 (10 mg/kg) | 957 ± 250 | 13.41 ± 3.24 | 69.07 | 33.87 | <0.001 | | |
| Cpd. 1 (0.3 mg/kg) | 198 ± 36 | 2.73 ± 0.37 | 14.06 | 90.53 | <0.001 | | |
| Cpd. 1 (0.3 mg/kg) + | 144 ± 30 | 2.08 ± 0.45 | 10.71 | 94.62 | <0.001 | <0.001 | >0.05 |
| Anti-PD-1 (10 mg/kg) | | | | | | | |
| Cpd. 1 (1 mg/kg) | 28 ± 7 | 0.40 ± 0.11 | 2.08 | 103.26 | <0.001 | | |
| Cpd. 1 (1 mg/kg) + | 27 ± 6 | 0.40 ± 0.09 | 2.06 | 103.32 | <0.001 | <0.001 | >0.05 |
| Anti-PD-1 (10 mg/kg) | | | | | | | |

^{a}Mean ± SEM;
^{b}All groups compare to G1;
^{c}Combination group compare to G2;
^{d}Combination group compare to Compound 1 monotherapy group

TABLE 2D-4
Survival Analysis

| | MST | ILS | p | p | p |
|---|---|---|---|---|---|
| Treatment | (day) | (%) | value^{a} | value^{b} | value^{c} |
| Vehicle control | 19 | — | — | | |
| Anti-PD-1 (10 mg/kg) | 20 | 5.3 | >0.05 | | |
| Compound 1 (0.3 mg/kg) | 29 | 52.6 | <0.001 | | |
| Compound 1 (0.3 mg/kg) + | 31.5 | 65.8 | <0.001 | <0.001 | >0.05 |
| Anti-PD-1 (10 mg/kg) | | | | | |
| Compound 1 (1 mg/kg) | 31.5 | 65.8 | <0.001 | | |
| Compound 1 (1 mg/kg) + | 33 | 73.7 | <0.001 | <0.001 | >0.05 |
| Anti-PD-1 (10 mg/kg) | | | | | |

^{a}All groups compare to G1;
^{b}Combination group compare to G2;
^{c}Combination group compare to Compound 1 monotherapy group

### Example 3

### In Vivo Test of Anti-Tumor Efficacy of Compound 1 in the Subcutaneous MC-38 Murine Colorectal Cancer Model in Female C57BL/6 Mice: Schedule Comparison

### I. Abstract

Female C57BL/6 mice were inoculated subcutaneously at right flank with MC-38 cells for tumor development. Six days after tumor inoculation, 64 mice with tumor size ranging from 50-100 mm³ (average tumor size 71 mm³) were selected and assigned into 8 groups using stratified randomization with 8 mice per group based upon their tumor volumes. The treatments were started from the day of randomization (defined as D0), Group 1 was treated with Vehicle (Tween 80:Ethanol:Saline—5:5:90 v/v/v), S.C. QD*20 days, Group 2 was treated with Compound 1 at 1 mpk S.C. QD*28 days, Group 3 was treated with Compound 1 at 0.5 mpk S.C. QD*28 days, Group 4 was treated with Compound 1 at 1.4 mpk S.C. 5-days ON 2-days OFF×4 cycles, Group 5 was treated with Compound 1 at 0.7 mpk S.C. 5-days ON 2-days OFF×4 cycles, Group 6 was treated with Compound 1 at 3 mpk qd×5, followed by 1 mpk qd×23, S.C. QD, Group 7 was treated with Compound 1 at 1.5 mpk qd×5, followed by 0.5 mpk qd×23, S.C. QD and Group 8 was treated with Compound 1 at 1.4 mpk 10-days ON 4-days OFF×2 cycles, S.C.

Four mice in Group 5 were tumor free on D67 and 1×10⁶ MC-38 cells suspended in 100 μL base DMEM were inoculated subcutaneously into the left flank. 5 naïve mice were inoculated with the same inoculation condition as control. The tumor sizes were measured three times per week during the treatment. The study was terminated on D102.

### II. Experimental Methods and Procedures

Species: Mus musculus; Strain: C57BL/6; Age: 6-8 weeks; Sex: female; Body weight (at treatment start): 16-20 g.

The MC-38 tumor cells were maintained in vitro as a monolayer culture in DMEM medium supplemented with 10% heat inactivated fetal bovine serum and 100 μg/mL penicillin streptomycin at 37° C. in an atmosphere of 5% CO₂ in air. The tumor cells were routinely subcultured twice weekly by trypsin-EDTA treatment. The cells growing to a confluency around 70%-80% were harvested and counted for tumor inoculation. The cultured MC-38 were harvested, re-suspended in base medium at a density of 1×10⁷ cells/mL with viability>90%. Each mouse was inoculated subcutaneously at the right flank with 1×10⁶ cells in 0.1 ml base medium for tumor development.

The treatments were started on day 6 after tumor inoculation when the tumor size reached 50-100 mm³ (average tumor size 71 mm³). Each group consisted of 8 tumor-bearing mice. The testing article was administrated to the mice at a dosing volume of 10 mL/Kg according to the predetermined regimen as shown in Table 3-1.

TABLE 3-1
Groups and Treatments for Efficacy Study

| | | Dose | |
|---|---|---|---|
| Group | Treatment | (mg/kg) | Dosing Route & Schedule |
| 1 | Vehicle control | — | qdx20, sc |
| 2 | Compound 1 | 1.0 | qdx28, sc |
| 3 | Compound 1 | 0.5 | qdx28, sc |
| 4 | Compound 1 | 1.4 | 5-days ON 2-days |
| | | | OFF x 4 cycles, sc |
| 5 | Compound 1 | 0.7 | 5-days ON 2-days |
| | | | OFF x 4 cycles, sc |
| 6 | Compound 1 | 3.0 −> 1.0 | 3 mpk qdx5, sc |
| | | | followed by 1 mpk, |
| | | | qdx23, sc |
| 7 | Compound 1 | 1.5 −>0.5 | 1.5 mpk qdx5, sc |
| | | | followed by 0.5 mpk, |
| | | | qdx23, sc |
| 8 | Compound 1 | 1.4 | 10-days ON 4-days |
| | | | OFF x 2 cycles, sc |

### Tumor Measurements and Endpoints

Tumor sizes were measured three times a week in two dimensions using a caliper, and the volume expressed in mm³ using the formula: V=0.5 a×b² where a and b are the long and short diameters of the tumor, respectively. The tumor sizes were then used for the calculations of both tumor growth inhibition (TGI) and T/C values.

TGI is calculated for each group using the formula:

$$TGI ( \% ) = [ 1 - ( {TV}_{Treatment \_ DayN} - {TV}_{Treatment \_ Day 0} ) / ( {TV}_{Verticle \_ DayN} - {TV}_{Vehicle \_ Day 0} ) ] \times 100 \% ;$$

TV_{Treatment_DayN} is the average tumor volume of a treatment group on a given day, TV_{Treatment_Day0} is the average tumor volume of the treatment group on the first day of treatment, TV_{Vehicle_DayN} is the average tumor volume of the vehicle control group on a given day, and TV_{Vehicle_Day0} is the average tumor volume of the vehicle group on the first day of treatment.

The T/C value (in percent) is an indication of antitumor effectiveness, T/C (%)=RTV_{Treatment}/RTV_{Control}×100% (RTV_{Treatment}: the mean RTV of the treatment group; RTV_{Control}: the mean RTV of the vehicle treated group). RTV (relative tumor volume)=TV_{DayN}/TV_{Day0}. TV_{DayN} and TV_{Day0} is the tumor volume on day N and Day 0 respectively. T/C (%)≤42% is considered as significant antitumor activity and <10% is considered as highly significant antitumor activity by the National Cancer Institute criteria.

Relative change of body weight (RCBW) of each mouse was calculated according to the following formula:

$$ILS ( \% ) = ( {MST}_{Treatment} - {MST}_{Vehicle} ) / {MST}_{Vehicle} \times 100 \% )$$

Animal survival curve: When an individual animal reached to the termination endpoint (TV>2000 mm³), the mouse was euthanized. The time from treatment initiation to the termination was deemed as its survival time. Survival curve was plotted by Kaplan-Meier method. Median survival time (MST) was calculated for each group. Increase of life span (ILS) was calculated according to the following formula:

$$RCBW ( \% ) = ( {BW}_{Treatment \_ DayN} - {BW}_{Treatment \_ Day 0} ) / {BW}_{Treatment \_ Day 0} \times 100 \% .$$

ILS (%)>25% is considered as a biologically significant survival benefit.

### Statistical Analysis

The tumor volume between different groups was analyzed by two-way repeated measures ANOVA. Dunnett post hoc test was used for the comparisons with vehicle group. All data analyzed using GraphPad Prism 6.0. P<0.05 is considered to be statistically significant.

### III. Results

### Body Weight Change

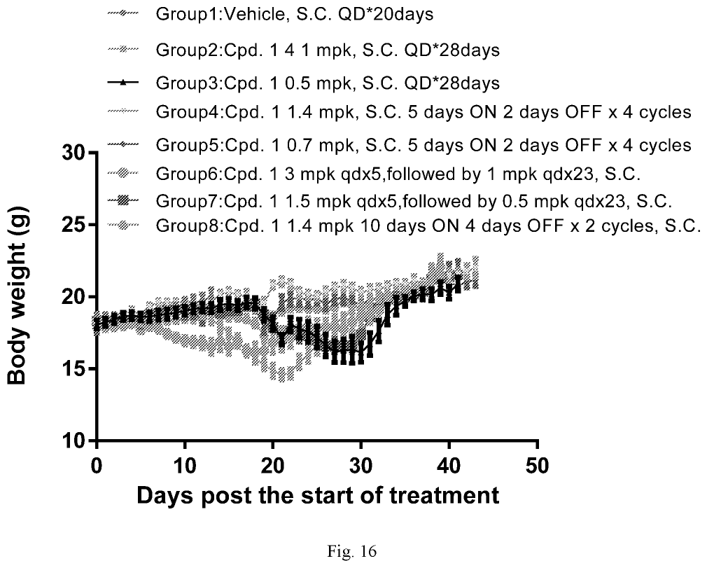

Body weight change in female C57BL/6 mice bearing MC-38 tumors are shown in FIG. 16. Once body weight loss was exceeding 15%, the treatment was suspended. After treatment suspension, the body weight recovered quickly

### Tumor Growth Analysis

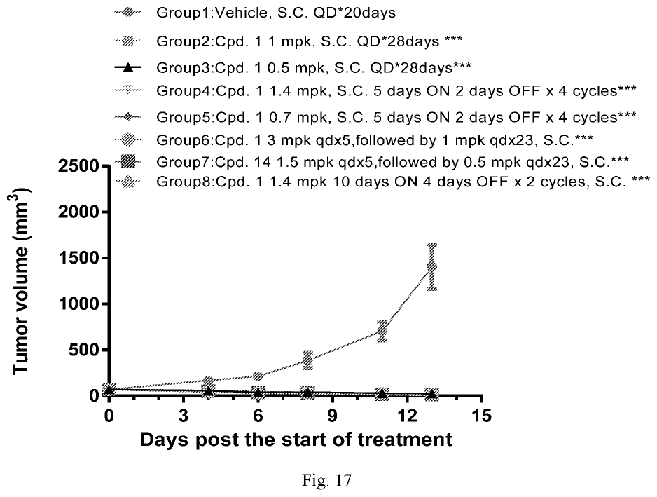

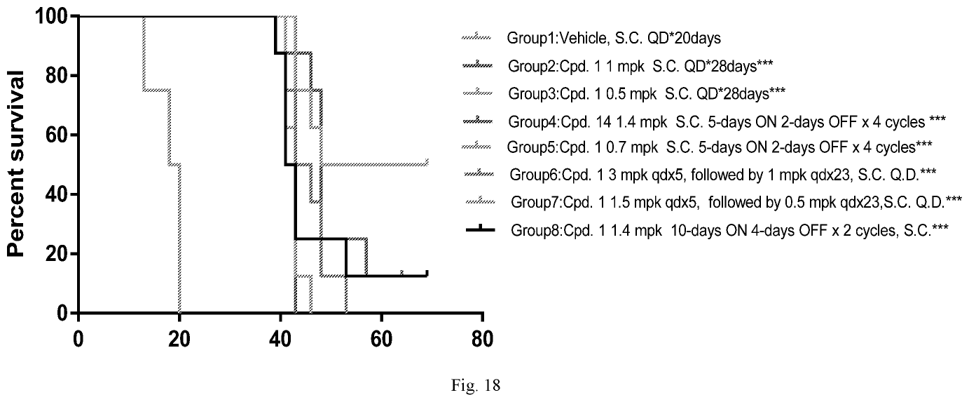

Mean tumor volume over time in female C57BL/6 mice bearing MC-38 tumors dosed with Compound 1 are shown in Table 3-3 and FIG. 17. The tumor growth inhibition analysis is shown in Table 3-4. The Kaplan-Meier survival curve (endpoint is defined as tumor volume reaching 2000 mm³) is shown in FIG. 18. The survival analysis is shown in Table 3-5.

Group 2 (1 mpk, QD), Group 6 (3 mpk, QD×5 followed by 1 mpk), two animals in Group 3 (0.5 mpk, QD), and two animals in Group 7 (1.5 mpk, qd×5 followed by 0.5 mpk) showed some body weight loss but the other treatments were well-tolerated without any adverse effects observed in the MC-38 tumor bearing C57BL/6 mice. Compared to the vehicle control group, all treatments showed significant inhibition on D13. Time-to-endpoint Kaplan-Meier survival analyses showed that all treatment groups showed significant survival benefits when compared to the vehicle (G1) group. Group 4 (1.4 mpk S.C. 5-days ON 2-days OFF×4 cycles) and Group 5 (0.7 mpk S.C. 5-days ON 2-days OFF×4 cycles) demonstrated superior and unexpected tolerability with no significant body weight loss while maintaining anti-tumor activity and inducing long term durable tumor free responders suggesting optimal treatment schedule for Compound 1. Four mice in Group 5 were completely tumor free on D67 and were termed “cured.” The same four mice were then re-implanted with MC-38 cells. Only three of the 4 mice regrew the tumor; and unexpectedly one mouse is still tumor free on Day 35 after re-implantation suggesting potential long-term immune memory effect of Compound 1 treatment in the MC-38 model.

TABLE 3-3
Tumor Volume over Time

| | Tumor volume (mm³)^{a} | | | | | | | |
|---|---|---|---|---|---|---|---|---|
| Days | Group 1 | Group 2 | Group 3 | Group 4 | Group 5 | Group 6 | Group 7 | Group 8 |
| 0 | 70 ± 5 | 70 ± 5 | 71 ± 5 | 71 ± 6 | 72 ± 6 | 72 ± 6 | 72 ± 6 | 72 ± 6 |
| 4 | 169 ± 32 | 45 ± 5 | 58 ± 6 | 51 ± 4 | 59 ± 6 | 48 ± 3 | 54 ± 3 | 57 ± 6 |
| 6 | 214 ± 36 | 34 ± 4 | 39 ± 3 | 45 ± 4 | 48 ± 5 | 29 ± 2 | 37 ± 3 | 41 ± 4 |
| 8 | 386 ± 80 | 25 ± 2 | 43 ± 4 | 30 ± 3 | 40 ± 7 | 31 ± 2 | 35 ± 3 | 26 ± 2 |
| 11 | 706 ± 101 | 15 ± 3 | 29 ± 3 | 14 ± 2 | 20 ± 4 | 24 ± 2 | 21 ± 2 | 28 ± 4 |
| 13 | 1402 ± 238 | 5 ± 2 | 24 ± 2 | 11 ± 3 | 13 ± 4 | 14 ± 3 | 15 ± 3 | 17 ± 4 |
| 15 | | 1 ± 1 | 17 ± 3 | 2 ± 1 | 6 ± 3 | 6 ± 2 | 9 ± 2 | 14 ± 3 |
| 18 | | 1 ± 0 | 10 ± 2 | 0 ± 0 | 3 ± 1 | 7 ± 3 | 8 ± 2 | 8 ± 3 |
| 20 | | 1 ± 0 | 7 ± 1 | 0 ± 0 | 2 ± 1 | 5 ± 2 | 5 ± 1 | 6 ± 2 |
| 22 | | 2 ± 1 | 6 ± 2 | 0 ± 0 | 2 ± 1 | 3 ± 1 | 6 ± 1 | 5 ± 2 |
| 25 | | 5 ± 1 | 6 ± 1 | 0 ± 0 | 2 ± 1 | 2 ± 1 | 4 ± 1 | 5 ± 2 |
| 27 | | 6 ± 2 | 7 ± 1 | 0 ± 0 | 1 ± 1 | 2 ± 1 | 4 ± 1 | 6 ± 2 |
| 29 | | 17 ± 11 | 13 ± 3 | 1 ± 1 | 2 ± 1 | 5 ± 3 | 10 ± 3 | 17 ± 5 |
| 32 | | 92 ± 47 | 45 ± 9 | 13 ± 7 | 20 ± 12 | 51 ± 35 | 44 ± 12 | 73 ± 19 |
| 34 | | 190 ± 81 | 121 ± 20 | 37 ± 21 | 32 ± 16 | 114 ± 79 | 112 ± 24 | 168 ± 43 |
| 36 | | 454 ± 212 | 285 ± 42 | 100 ± 47 | 125 ± 63 | 288 ± 174 | 364 ± 69 | 479 ± 121 |
| 39 | | 1109 ± 398 | 592 ± 61 | 238 ± 72 | 242 ± 114 | 723 ± 345 | 904 ± 174 | 945 ± 260 |
| 41 | | | 1718 ± 136 | 420 ± 129 | 443 ± 200 | | 1829 ± 275 | |
| 43 | | | | 847 ± 249 | 817 ± 381 | | | |

^{a}Mean ± SEM;
n = 8

TABLE 3-4
Tumor Growth Inhibition Calculation Based on TV Measurements at Day 13

| | Tumor Size | | | | |
|---|---|---|---|---|---|
| | (mm³)^{a} | RTV | T/C | | |
| Treatment | at day 13 | at day 13 | (%) | TGI (%) | p value^{b} |
| Vehicle QD*20 days | 1402 + 238 | 20 ± 3 | — | — | — |
| Compound 1 1 mpk QD*28 days | 5 + 2 | 0 ± 0 | 0 | 104.87 | <0.001 |
| Compound 1 0.5 mpk QD*28 days | 24 + 2 | 0 ± 0 | 0 | 103.48 | <0.001 |
| Compound 1 1.4 mpk days ON | 11 + 3 | 0 ± 0 | 0 | 104.49 | <0.001 |
| 2-days OFF x 4 cycles | | | | | |
| Compound 1 0.7 mpk S.C. 5-days ON | 13 + 4 | 0 ± 0 | 0 | 104.42 | <0.001 |
| 2-days OFF | | | | | |
| Compound 1 3 mpk qdx5, | 14 + 3 | 0 ± 0 | 0 | 104.29 | <0.001 |
| followed by 1 mpk qdx23 | | | | | |
| Compound 1 1.5 mpk qdx5, | 15 + 3 | 0 ± 0 | 0 | 104.24 | <0.001 |
| followed by 0.5 mpk qdx23 | | | | | |
| Compound 1 1.4 mpk 10-days ON | 17 + 4 | 0 ± 0 | 0 | 104.10 | <0.001 |
| 4-days OFF | | | | | |

^{a}Mean ± SEM;
^{b}All groups compare to G1

TABLE 3-5
Survival Analysis

| | MST | ILS | p |
|---|---|---|---|
| Treatment | (day) | (%) | value^{a} |
| Vehicle QD*20 days | 19 | — | — |
| Compound 1 1 mpk QD*28 days | 43 | 126 | <0.001 |
| Compound 1 0.5 mpk QD*28 days | 43 | 126 | <0.001 |
| Compound 1 1.4 mpk days ON | 48 | 153 | <0.001 |
| 2-days OFF x 4 cycles | | | |
| Compound 1 0.7 mpk S.C. | 58.5 | 208 | <0.001 |
| 5-days ON 2-days OFF | | | |
| Compound 1 3 mpk qdx5, | 44.5 | 134 | <0.001 |
| followed by 1 mpk qdx23 | | | |
| Compound 1 1.5 mpk qdx5, | 42 | 121 | <0.001 |
| followed by 0.5 mpk qdx23 | | | |
| Compound 1 1.4 mpk 10-days | 42 | 121 | <0.001 |
| ON 4-days OFF | | | |

^{a}All groups compare to G1

### Results of Re-Implantation Study

Four mice in Group 5 were tumor free on D67 and 1×10⁶ MC-38 cells suspended in 100 μL base DMEM were inoculated subcutaneously into the left flank. 5 naïve mice were inoculated with the same inoculation condition as control. Mean tumor volume over time in re-implanted female C57BL/6 mice bearing MC-38 tumors are shown in Table 3-6.

TABLE 3-6
Tumor Volume over Time

Tumor volume (mm³)^{a}

| | Group1 | Group2 |
|---|---|---|
| Days | Naïve | Re-implantation |
| 7 | 42 ± 8 | 32 ± 14 |
| 9 | 166 ± 37 | 106 ± 44 |
| 11 | 292 ± 71 | 152 ± 55 |
| 14 | 499 ± 112 | 373 ± 174 |
| 16 | 657 ± 125 | 568 ± 258 |
| 18 | 1189 ± 259 | 886 ± 429 |
| 21 | | 1911 ± 794 |

^{a}Mean ± SEM;

### Example 4

### In Vivo Test of Anti-Tumor Efficacy of Compound 1 with and without Anti-PD-1 in the Subcutaneous MC-38 Murine Colorectal Cancer Model in Female C57BL/6

### I. Abstract

Female C57BL/6 mice were inoculated subcutaneously at right flank with MC-38 cells for tumor development. Six days after tumor inoculation, 64 mice with tumor size ranging from 50-100 mm³ (average tumor size 71 mm³) were selected and assigned into 8 groups using stratified randomization with 8 mice per group based upon their tumor volumes. The treatments were started from the day of randomization (defined as D0), Group 1 was treated with Vehicle (Tween 80:Ethanol:Saline—5:5:90 v/v/v), s.c., qd×14, Group 2 was treated with Anti-PD-1 10 mg/kg i.p. q4d×4, Group 3 was treated with Compound 1 3 mpk qd×5, sc followed by 1 mpk, qd×9, sc, Group 4 was treated with Compound 1 3 mpk qd×5, sc followed by 1 mpk, qd×9, sc+anti PD-1 q4d×4, ip, Group 5 was treated with Compound 1 1 mpk qd×5, sc followed by 0.3 mpk, qd×9, sc, Group 6 was treated with Compound 1 1 mpk qd×5, sc followed by 0.3 mpk, qd×9, sc+anti PD-1 q4d×4, ip, Group 7 was treated with Compound 1 0.3 mpk qd×5, sc followed by 0.1 mpk, qd×9, sc and Group 8 was treated with Compound 1 0.3 mpk qd×5, sc followed by 0.1 mpk, qd×9, sc+anti PD-1 q4d×4, ip. The tumor sizes were measured three times per week during the treatment. The entire study was terminated on D56 after start of the treatment.

### II. Experimental Methods and Procedures

Animal Species: Mus musculus; Strain: BALB/c; Age: 6-8 weeks; Sex: female; Body weight (at treatment start): 16-20 g.

The MC-38 tumor cells were maintained in vitro as a monolayer culture in DMEM medium supplemented with 10% heat inactivated fetal bovine serum and 100 μg/mL penicillin streptomycin at 37° C. in an atmosphere of 5% CO₂ in air. The tumor cells were routinely subcultured twice weekly by trypsin-EDTA treatment. The cells growing to a confluency around 70%-80% were harvested and counted for tumor inoculation. The cultured MC-38 were harvested, re-suspended in base medium at a density of 1×10⁷ cells/mL with viability >90%. Each mouse was inoculated subcutaneously at the right flank with 1×10⁶ cells in 0.1 ml base medium for tumor development.

The treatments were started on day 6 after tumor inoculation when the tumor size reached 50-100 mm³ (average tumor size 71 mm³). Each group consisted of 8 tumor bearing mice. The testing article was administrated at the dosing volume indicated to the mice according to the predetermined regimen as shown Table 4-1. Testing article formulations were prepared according to standard procedures. Details regarding tumor measurements and endpoints, and statistical analysis are provided in EXAMPLE 3.

TABLE 4-1
Groups and Treatments for Efficacy Study

| | | Dose | |
|---|---|---|---|
| Group | Treatment | (mg/kg) | Dosing Route & Schedule |
| 1 | Vehicle control | 10 ml/kg | qdx14, sc |
| 2 | anti-PD-1 | 10 | q4dx4, ip |
| 3 | Cpd. 1 | 3.0 → 1.0 | 3 mpk qdx5, sc followed |
| | | | by 1 mpk, qdx9, sc |
| 4 | Cpd. 1 + | 3.0 → 1.0 + | 3 mpk qdx5, sc followed |
| | anti-PD-1 | 10 (aPD-1) | by 1 mpk, qdx9, sc |
| | | | q4dx4, ip |
| 5 | Cpd. 1 | 1.0 → 0.3 | 1 mpk qdx5, sc followed |
| | | | by 0.3 mpk, qdx9, sc |
| 6 | Cpd. 1 + | 1.0 → 0.3 + | 1 mpk qdx5, sc followed |
| | anti-PD-1 | 10 (aPD-1) | by 0.3 mpk, qdx9, sc |
| | | | q4dx4, ip |
| 7 | Cpd. 1 | 0.3 → 0.1 | 0.3 mpk qdx5, sc followed |
| | | | by 0.1 mpk, qdx9, sc |
| 8 | Cpd. 1 + | 0.3 → 0.1 + | 0.3 mpk qdx5, sc followed |
| | anti-PD-1 | 10 (aPD-1) | by 0.1 mpk, qdx9, sc |
| | | | q4dx4, ip |

### III. Results

### Body Weight Change

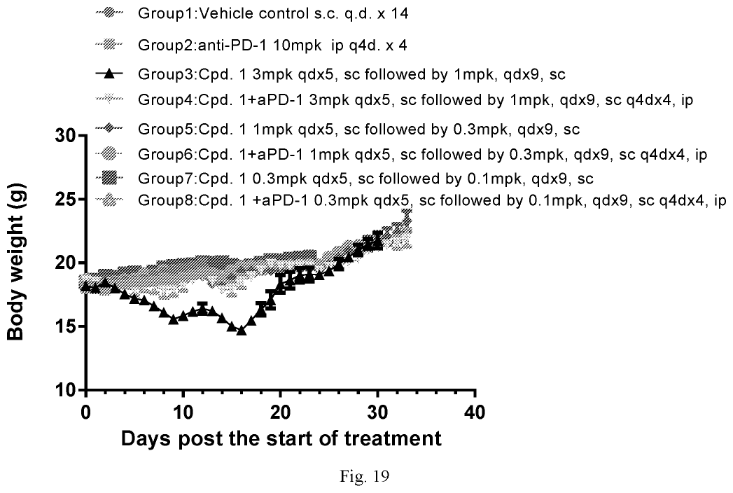

Body weight change in female C57BL/6 mice bearing MC-38 tumors are shown in FIG. 19. Once body weight loss was exceeding 15%, the treatment was suspended. After treatment suspension, the body weight recovered quickly.

### Tumor Growth Analysis

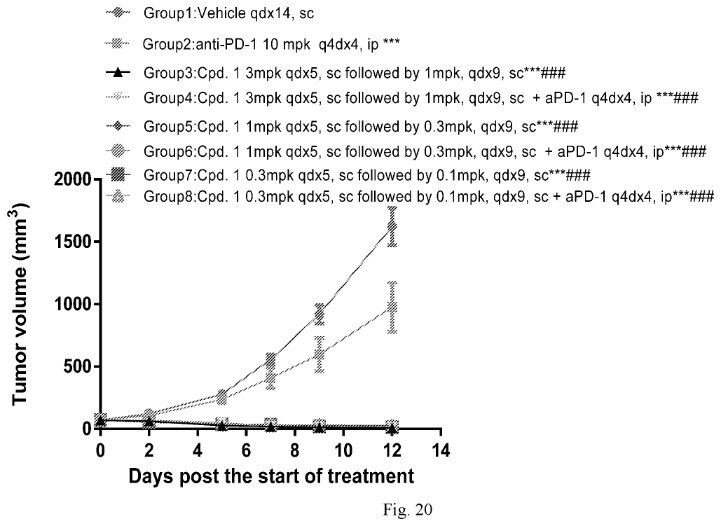

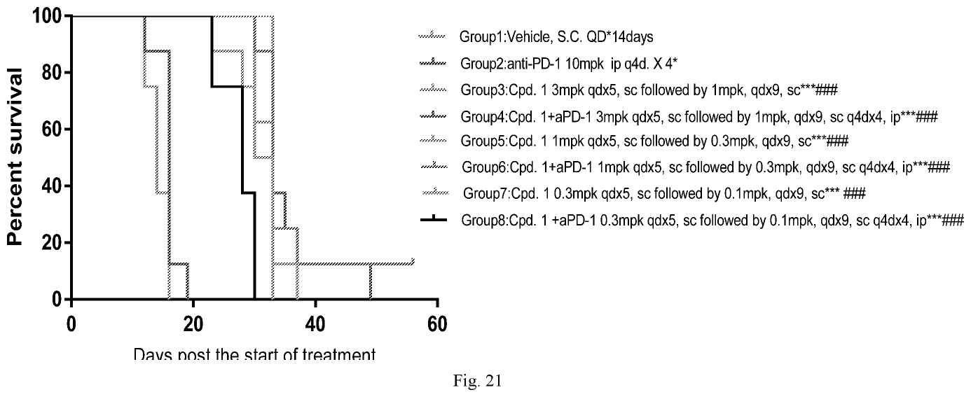

Mean tumor volume over time in female C57BL/6 mice bearing MC-38 tumors dosed with Anti-PD-1 (aPD-1) and Compound 1 is shown in Table 4-2 and FIG. 20. The tumor growth inhibition analysis is shown in Table 4-3. The Kaplan-Meier survival curve (endpoint is defined as tumor volume reaching 2000 mm³) is shown in FIG. 21. The survival analysis is shown in Table 4-4.

Group 3 induced some body weight loss but the other treatments were well-tolerated without any adverse effects observed in the MC-38 tumor bearing C57BL/6 mice. Compared to the vehicle control group, all treatments showed significant inhibition on D12. Time-to-endpoint Kaplan-Meier survival analyses showed that all treatment groups showed significant survival benefits when compared to the vehicle (G1) group.

TABLE 4-2
Tumor Volume over Time

| | Tumor volume (mm³)^{a} | | | | | | | |
|---|---|---|---|---|---|---|---|---|
| | | | | Cpd. 1 + | | Cpd. 1 + | | Cpd. 1 + |
| | | | | aPD-1 | | aPD-1 | Cpd. 1 | aPD-1 |
| | | aPD-1 | Cpd. 1 | 3/1 mpk + | Cpd. 1 | 1/0.3 mpk + | 0.3/0.1 | 0.3/0.1 mpk + |
| Days | Vehicle | 10 mg/kg | 3/1 mpk | 10 mpk | 1/0.3 mpk | 10 mpk | mpk | 10 mpk |
| 0 | 70 ± 4 | 70 ± 5 | 70 ± 4 | 70 ± 6 | 71 ± 5 | 71 ± 6 | 72 ± 5 | 72 ± 5 |
| 2 | 123 ± 11 | 106 ± 13 | 63 ± 7 | 47 ± 6 | 67 ± 5 | 66 ± 4 | 86 ± 11 | 82 ± 4 |
| 5 | 277 ± 20 | 236 ± 32 | 25 ± 4 | 25 ± 5 | 38 ± 3 | 33 ± 4 | 72 ± 6 | 69 ± 6 |
| 7 | 558 ± 40 | 408 ± 81 | 19 ± 3 | 17 ± 5 | 27 ± 5 | 31 ± 3 | 70 ± 9 | 70 ± 13 |
| 9 | 917 ± 74 | 596 ± 134 | 12 ± 2 | 11 ± 4 | 25 ± 4 | 31 ± 2 | 76 ± 12 | 92 ± 20 |
| 12 | 1622 ± 150 | 975 ± 198 | 8 ± 2 | 9 ± 3 | 27 ± 7 | 25 ± 6 | 114 ± 25 | 154 ± 42 |

^{a}Mean ± SEM;
n = 8

TABLE 4.3
Tumor Growth Inhibition Calculation Based on TV Measurements at Day 12

| | Tumor Size | | | | | | |
|---|---|---|---|---|---|---|---|
| | (mm³)^{a} | RTV | T/C | TGI | p | p | p |
| Treatment | at day 12 | at day 12 | (%) | (%) | value^{b} | value^{c} | value^{d} |
| Vehicle | 1622 ± 150 | 23.79 ± 2.86 | — | — | — | | |
| Anti-PD-1(10 mg/kg) | 975 ± 198 | 13.55 ± 2.30 | 56.96 | 41.67 | <0.001 | | |
| Cpd. 1 (3 mpk/1 mpk) | 8 ± 2 | 0.11 ± 0.03 | 0.46 | 103.98 | <0.001 | | |
| Anti-PD-1 (10 mg/kg) + | 9 ± 3 | 0.12 ± 0.04 | 0.50 | 103.96 | <0.001 | <0.001 | >0.05 |
| Cpd. 1 (3 mpk/1 mpk) | | | | | | | |
| Cpd. 1 (1 mpk/0.3 mpk) | 27 ± 7 | 0.36 ± 0.09 | 1.51 | 102.87 | <0.001 | | |
| Anti-PD-1(10 mg/kg) + | 25 ± 6 | 0.46 ± 0.15 | 1.93 | 103.01 | <0.001 | <0.001 | >0.05 |
| Cpd. 1 (1 mpk/0.3 mpk) | | | | | | | |
| Cpd. 1 (0.3 mpk/0.1 mpk) | 114 ± 25 | 1.67 ± 0.03 | 7.02 | 97.27 | <0.001 | | |
| Cpd. 1 (0.3 mpk/0.1 mpk) + | 154 ± 42 | 2.27 ± 0.7 | 9.54 | 94.67 | <0.001 | <0.001 | >0.05 |
| Anti-PD-1 (10 mg/kg) | | | | | | | |

^{a}All groups compare to G1;
^{b}Combination group compare to G2;
^{c}Combination group compare to Compound 1 monotherapy group

TABLE 4.4
Survival Analysis

| | MST | ILS | p | p | p |
|---|---|---|---|---|---|
| Treatment | (day) | (%) | value^{b} | value^{c} | value^{d} |
| Vehicle | 14 | — | — | | |
| Anti-PD-1 (10 mg/kg) | 16 | 14.29 | <0.05 | | |
| Cpd. 1 (3 mpk/1 mpk) | 33 | 135.71 | <0.001 | | |
| Anti-PD-1 (10 mg/kg) + | 33 | 135.71 | <0.001 | <0.001 | <0.05 |
| Cpd. 1 (3 mpk/1 mpk) | | | | | |
| Cpd. 1 (1 mpk/0.3 mpk) | 33 | 135.71 | <0.001 | | |
| Anti-PD-1 (10 mg/kg) + | 33 | 135.71 | <0.001 | <0.001 | >0.05 |
| Cpd. 1 (1 mpk/0.3 mpk) | | | | | |
| Cpd. 1 (0.3 mpk/0.1 mpk) | 30 | 114.29 | <0.001 | | |
| Cpd. 1 (0.3 mpk/0.1 | 28 | 100 | <0.001 | <0.001 | <0.05 |
| mpk) + Anti-PD-1(10 | | | | | |
| mg/kg) | | | | | |

### Example 5

### In Vivo Anti-Tumor Efficacy of Compound 1 in the CT26.WT Murine Colorectal Model in Female BALB/c Mice: Schedule Comparison

Female BALB/c mice were inoculated subcutaneously at right flank with CT26.WT cells for tumor development. Treatment started on day 6 after tumor inoculation when the tumor size reached 40-79 mm³ (average tumor size 57 mm³). Each group consisted of 8 tumor-bearing mice. Compound 1 was administrated to the mice according to the predetermined regimen as shown in Table 5-1. The tumor sizes were measured three times per week during the treatment. The entire study was terminated on D57 after start of the treatment. Otherwise, the experimental methods and procedures are similar to those described in EXAMPLE 2B.

TABLE 5-1
Groups and Treatments for Efficacy Study

| | | Dose | |
|---|---|---|---|
| Group | Treatment | (mg/kg) | Dosing Route & Schedule |
| 1 | Vehicle | 10 | qdx21, sc |
| | control | | |
| 2 | Compound 1 | 1.4 | 1.4 mpk 5-days ON (Mon-Fri) |
| | | | 2-days OFF (Sat-Sun) x4 cycles, sc |
| 3 | Compound 1 | 1.4 | 1.4 mpk 5-days ON (Mon-Fri) |
| | | | 2-days OFF (Sat-Sun) x2 cycles, sc |
| | | | followed by |
| | | | 1.4 mpk 3-days ON (Mon-Wed) |
| | | | 4-days OFF (Thur-Sun) x2 cycles, sc |
| 4 | Compound 1 | 1.4 | 1.4 mpk 5-days ON (Mon-Fri) |
| | | | 2-days OFF (Sat-Sun) x2 cycles, sc |
| | | | followed by |
| | | | 1.4 mpk 3-days ON (Mon, Wed, Fri) |
| | | | 4-days OFF (Tues, Thur, Sat, Sun) x2 |
| | | | cycles, sc |
| 5 | Compound 1 | 2.33 | 2.33 mpk 3-days ON (Mon-Wed) |
| | | | 4-days OFF (Thur-Sun) x4 cycles, sc |
| 6 | Compound 1 | 2.33 | 2.33 mpk 3-days ON (Mon, Wed, Fri) |
| | | | 4-days OFF (Tue, Thur, Sat, Sun) x4 |
| | | | cycles, sc |
| 7 | Compound 1 | 0.5 | 0.5 mpk 5-days ON (Mon-Fri) |
| | | | 2-days OFF (Sat-Sun) x4 cycles, sc |
| 8 | Compound 1 | 0.83 | 0.83 mpk 3-days ON (Mon-Wed) |
| | | | 4-days OFF (Thur-Sun) x4 cycles, sc |
| 9 | Compound 1 | 0.83 | 0.83 mpk 3-days ON (Mon, Wed, Fri) |
| | | | 4-days OFF (Tue, Thur, Sat, Sun) x4 |
| | | | cycles, sc |

The mean tumor volume over time, tumor growth inhibition analysis, and survival analysis in female BALB/c mice bearing CT26.WT tumors dosed with Compound 1 is shown in Table 5-2. All treatment groups showed significant tumor growth inhibition (p<0.001) on Day 14 compared to the vehicle control.

TABLE 5-2

| | Mean Tumor Vol | TGI | | ILS | | Cures |
|---|---|---|---|---|---|---|
| Group | (mm³)^{a} | (%)^{a} | p-Value | (%) | p-Value | (%) |
| 1 | 1420 ± 278 | NA | NA | NA | NA | NA |
| 2 | 163 ± 25 | 92 | <0.001 | 106 | <0.001 | 12.5 |
| 3 | 155 ± 27 | 93 | <0.001 | 67 | <0.001 | 0 |
| 4 | 164 ± 12 | 92 | <0.001 | 94 | <0.001 | 12.5 |
| 5 | 169 ± 32 | 92 | <0.001 | 86 | <0.001 | 0 |
| 6 | 240 ± 45 | 87 | <0.001 | 94 | <0.001 | 0 |
| 7 | 302 ± 41 | 82 | <0.001 | 67 | <0.001 | 0 |
| 8 | 484 ± 87 | 69 | <0.001 | 67 | <0.001 | 0 |
| 9 | 488 ± 74 | 68 | <0.001 | 39 | <0.001 | 0 |

^{a}Determined on Day 14;
NA: not applicable

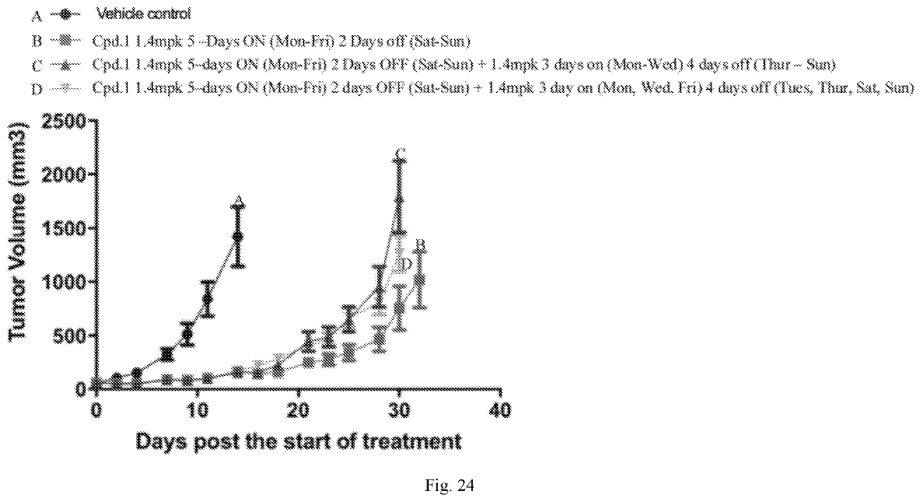

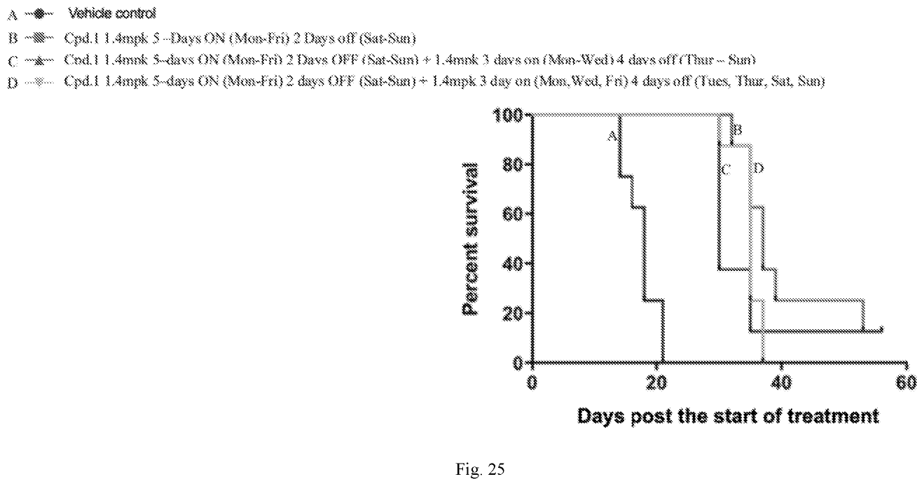

Comparing schedules at the same dose of 1.4 mg/kg, shows that switching from 5-day ON 2-day OFF schedule to 3-day ON 4-day OFF schedule on Day 14 results in reduced tumor growth inhibition as compared to 1.4 mg/kg 5-day ON 2-day OFF×4 cycles. Compound 1 dosed at 1.4 mg/kg 5-day ON 2-day OFF achieved maximum median survival (106% ILS) including 1/8 long term cure. See FIG. 24 and FIG. 25 Tolerability was similar amongst groups based on relative change in body weight.

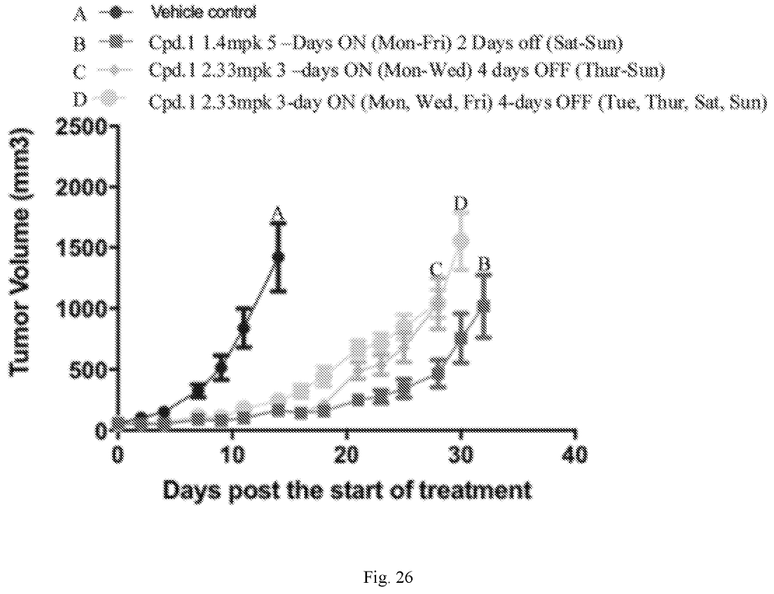

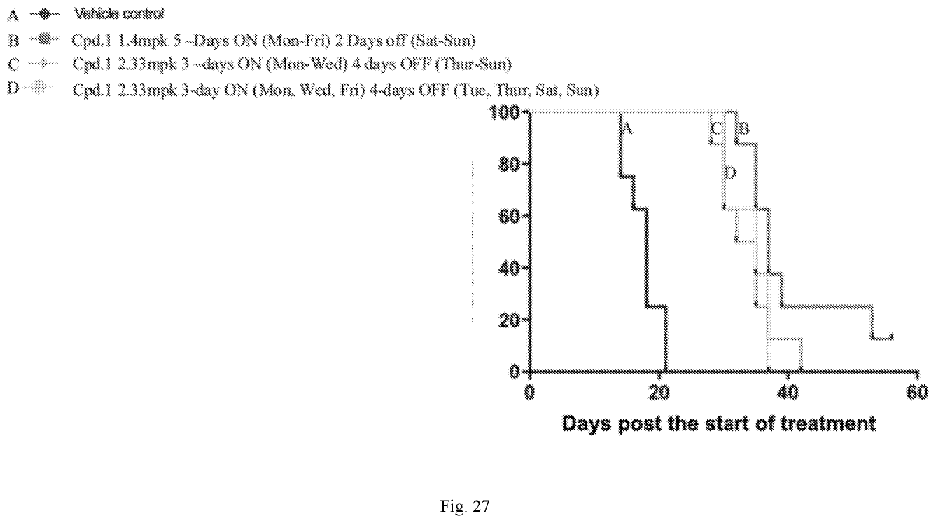

Compound 1 was dosed on 5-day ON 2-day OFF or 3-day ON 4-day OFF schedules for 4 weeks, including a 3-day ON 4-day OFF schedule of Mon-Wed or Mon, Wed, Fri dosing. These groups were included to compare an equivalent total dose per cycle. The 3-day ON 4-day OFF schedules Mon-Wed or Mon, Wed, Fri dosing schedules demonstrated equivalent efficacy and survival benefit. Unexpectedly, the 5-day ON 2-day OFF schedule showed superior tumor growth inhibition and survival benefit as compared to 3-day ON 4-day OFF schedules in this model. See FIG. 26 and FIG. 27.

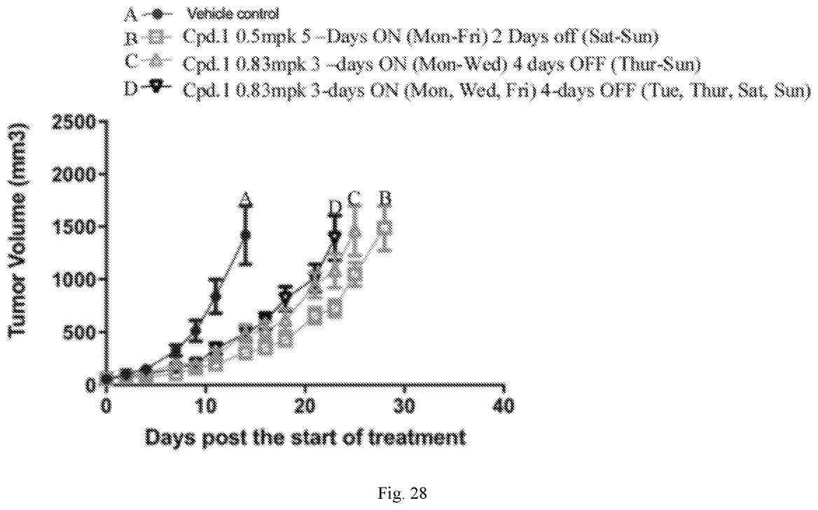

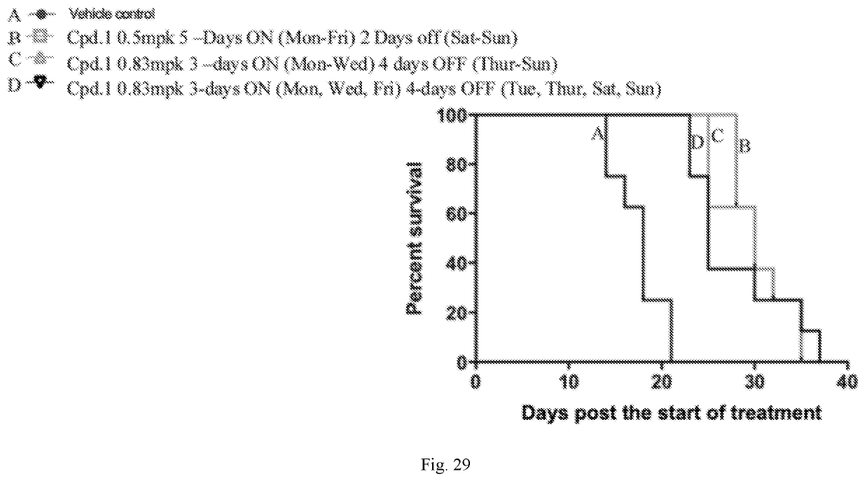

As was observed at the higher doses, the 5-day ON 2-day OFF schedule maintains improvement in tumor growth inhibition even at the lower dose levels as compared to 3-day ON 4-day OFF schedules but shows similar survival outcome. At lower dose levels that more frequent dosing provides improved tumor growth inhibition. See FIG. 28 and FIG. 29.

### Example 6

### In Vivo Anti-Tumor Efficacy of Compound 1 with and without Anti-PD-L1 in the H22 Subcutaneous Syngeneic Model in Balb/C Mice

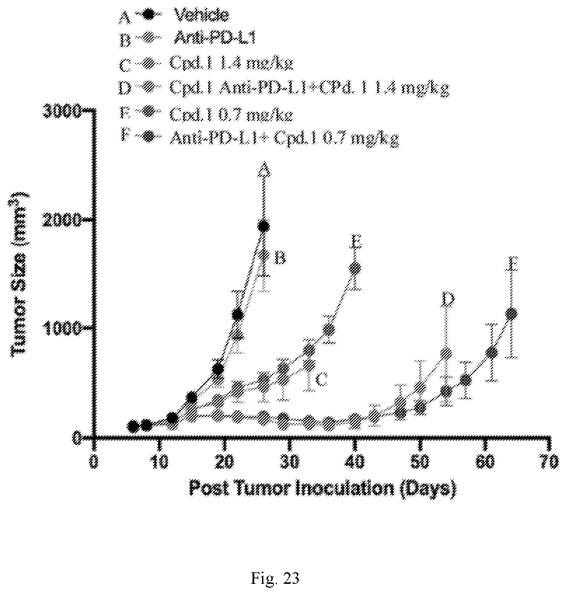

Female BALB/c mice were inoculated subcutaneously at right flank with H22 tumor cells for tumor development. Treatment began on Day 6 post tumor inoculation and the mice were treated for 21 days. Tumor and weight measurements were continued for the duration of the study, and the survival was recorded until Day 106. The results are summarized Table 6-1, Table 6-2, and FIG. 23. In all cases the dosing volume was 10 ml/kg. PD-L1 antibody was obtained from Bioxcell.

TABLE 6-1

| | | | | Mean | | | | |
|---|---|---|---|---|---|---|---|---|
| | | | | Tumor | | p-Value | p-Value | p-Value |
| Test | Dose | Route and | BWC | Vol | TGI | vs | vs Anti- | vs |
| Article | (mg/kg) | Schedule | (%)^{a} | (mm³)^{b} | (%)^{b} | Vehicle | PD-L1 | Cpd. 1 |
| Vehicle | — | | 15 | 2127 ± 455 | NA | NA | NA | NA |
| Anti- | 5 | Q4Dx6 | 14 | 1666 ± 331 | 23 | <0.01 | NA | NA |
| PD-L1 | | | | | | | | |
| Cpd. 1 | 0.7 | 5-day ON 2- | 0 | 529 ± 64 | 79 | <0.001 | NA | NA |
| | | day OFF x 3 | | | | | | |
| | | cycles | | | | | | |
| Cpd. 1 + | 0.7 + 5 | 5-day ON 2- | 6 | 194 ± 14 | 96 | <0.001 | <0.001 | <0.001 |
| anti-PD- | | day OFF x 3 | | | | | | |
| L1 | | cycles + | | | | | | |
| | | Q4D x 6 | | | | | | |
| Cpd. 1 | 1.4 | 5-day ON 2- | 7 | 465 ± 133 | 82 | <0.001 | NA | NA |
| | | day OFF x 3 | | | | | | |
| | | cycles | | | | | | |
| Cpd. 1 + | 1.4 ± 5 | 5-day ON 2- | 5 | 166 ± 21 | 97 | <0.001 | <0.001 | <0.001 |
| anti-PD- | | day OFF x 3 | | | | | | |
| L1 | | cycles + | | | | | | |
| | | Q4D x 6 | | | | | | |

^{a}Determined on Day 26;
^{b}Determined on Day 26;
NA: not applicable

TABLE 6-2

| | | | | p-Value | p-Value | p-Value | |
|---|---|---|---|---|---|---|---|
| Test | Dose | Route and | MST | vs | vs Anti- | vs | Cure |
| Article | (mg/kg) | Schedule | (days) | Vehicle | PD-L1 | Cpd. 1 | Rate (%) |
| Vehicle | — | | 28 | NA | NA | NA | 0 |
| Anti- | 5 | Q4D x 6 | 30 | >0.05 | NA | NA | 0 |
| PD-L1 | | | | | | | |
| Cpd. 1 | 0.7 | 5-day ON 2- | 45 | <0.01 | NA | NA | 0 |
| | | day OFF x 3 | | | | | |
| | | cycles | | | | | |
| Cpd. 1 + | 0.7 + 5 | 5-day ON 2- | 77 | <0.001 | <0.001 | <0.001 | 12.5 |
| anti-PD- | | day OFF x 3 | | | | | |
| L1 | | cycles + Q4D | | | | | |
| | | x 6 | | | | | |
| Cpd. 1 | 1.4 | 5-day ON 2- | 47 | <0.01 | NA | NA | 0 |
| | | day OFF x 3 | | | | | |
| | | cycles | | | | | |
| Cpd. 1 + | 1.4 + 5 | 5-day ON 2- | 96 | <0.001 | <0.001 | <0.001 | 50 |
| anti-PD- | | day OFF x 3 | | | | | |
| L1 | | cycles + Q4D | | | | | |
| | | x 6 | | | | | |

Treatment with anti-PD-L1 alone at 5 mg/kg biweekly had minimal tumor growth inhibition effect in this model. Compound 1 treatment alone demonstrated significant antitumor activity with TGI of ˜80% (p<0.001) at both 0.7 and 1.4 mg/kg. The combination of Compound 1 and anti-PD-L1 demonstrated statistically significant greater anti-tumor activity as compared to either anti-PD-L1 or Compound 1 monotherapy with a TGI of ˜96%0 (p<0.001) for both combination groups, respectively. This synergistic antitumor activity of the combination of Compound 1 and anti-PD-L1 also led to an unexpected increased survival benefit of 76 and 96 days for the 0.7 and 1.4 mg/kg combination groups, respectively, which was statistically significant compared to both the anti-PD-L1 alone (<0.001) and Compound 1 alone (<0.001). Combination treatment resulted in a surprising 12.5% and 50% long-term durable cures in the 0.7 mg/kg and 1.4 mg/kg combination groups, respectively.

### Example 7

### Effects of Compound 1 and DON on Cell Viability

### Experiment 1

Cells were cultured in the appropriate medium at 37° C. and 5% CO₂ atmosphere. Cells were harvested by trypsinization, spun at 800 rpm for 5 min and resuspended in medium. The cell concentration was adjusted with medium and seeded at a cell density of 3000 cells in 90 μL per well in 96-well plates, and incubated overnight at 37° C. and 5% CO₂. After 24 hours, the 1 mM test compound (either Compound 1 or DON) was successively diluted into 7 concentrations according to a three-fold gradient respectively, and 10 μl/well drug was added. The final concentrations were 100 μM, 33.33 μM, 11.11 μM, 3.7 μM, 1.24 μM, 0.41 μM, 0.14 μM, and 0.046 μM. The 100 μM Positive Control drug was successively diluted in 7 concentrations according to a three-fold gradient, and 10 μl/well drug was added. The final concentrations were 10 μM, 3.333 μM, 1.111 μM, 0.37 μM, 0.124 μM, 0.041 μM, 0.014 μM and 0.0046 μM. Cells were incubated for 72 h at 37° C. and 5% CO₂. 100 μL CTG reagent was then added to each well. Plates were shaken for 2 min and placed 10 min at room temperature. Luminescence was recorded on Perkin Elmer Envision 2104 Multilabel Reader. IC₅₀'s were obtained by fitting curve using GraphPad Prism5 software. Results are provided in Table 7.1. In all cases the treatment time was 72 h.

TABLE 7.1

| | | | Positive Control |
|---|---|---|---|
| Cell lines | Test Compound | IC₅₀ (μM) | IC₅₀ (μM) |
| NCI-H322M | Compound 1 | 49.12 | 7.142 |
| | DON | 4.783 | 12.12 |
| 769-P | Compound 1 | 2.079 | 3.547 |
| | DON | 1.851 | 3.443 |
| Capan-1 | Compound 1 | 29.31 | 5.199 |
| | DON | 67.47 | 2.906 |
| NCI-H23 | Compound 1 | 3.531 | 1.196 |
| | DON | 1.658 | 1.186 |
| NCI-H661 | Compound 1 | 130.8 | 7.08 |
| | DON | 70.61 | 9.347 |
| NCI-H1299 | Compound 1 | 9.195 | 3.954 |
| | DON | 1.819 | 6.141 |
| NCI-H1650 | Compound 1 | 6.608 | 2.414 |
| | DON | 1.887 | 2.092 |
| SNB-19 | Compound 1 | 3.208 | 1.962 |
| | DON | 2.027 | 1.736 |
| ZR-75-1 | Compound 1 | 136.8 | 29.61 |
| | DON | 101.4 | 31.26 |
| Malme-3M | Compound 1 | 19.91 | 9.984 |
| | DON | 36.65 | 12.91 |
| SNU-449 | Compound 1 | 38.6 | 28.17 |
| | DON | 2.18 | 21.42 |
| KATO-III | Compound 1 | 10.7 | 4.237 |
| | DON | 2.96 | 3.171 |
| MOLT-4 | Compound 1 | 3.03 | 1.563 |
| | DON | 2.384 | 1.186 |
| HCC1395 | Compound 1 | 59.83 | 1.273 |
| | DON | 32.27 | 1.082 |
| HCC1806 | Compound 1 | 3.859 | 0.8147 |
| | DON | 0.7199 | 0.8411 |
| NCI-H929 | Compound 1 | 13.77 | 0.4291 |
| | DON | 11.76 | 0.4238 |
| HCC1954 | Compound 1 | 3.327 | 1.934 |
| | DON | 0.9506 | 1.479 |
| MDA-MB-231 | Compound 1 | 2.881 | 0.6888 |
| | DON | 1.783 | 0.7741 |
| AGS | Compound 1 | 19.85 | 1.663 |
| | DON | 13.8 | 1.39 |
| TT | Compound 1 | 77.11 | 2.174 |
| | DON | 42.86 | 1.549 |
| T98G | Compound 1 | 3.387 | 0.8885 |
| | DON | 1.375 | 0.9272 |
| C3A | Compound 1 | 8.392 | 7.662 |
| | DON | 2.403 | 6.522 |
| 143B | Compound 1 | 2.438 | 2.34 |
| | DON | 1.06 | 2.23 |
| RKO | Compound 1 | 3.644 | 1.298 |
| | DON | 3.222 | 1.061 |
| LN-18 | Compound 1 | 4.149 | 2.211 |
| | DON | 2.71 | 3.146 |
| SW480 | Compound 1 | 88.09 | 6.352 |
| | DON | 100.2 | 8.367 |
| ZR-75-30 | Compound 1 | 282 | Not converged |
| | DON | 981.8 | 97.03 |
| SK-MES-1 | Compound 1 | 71.55 | 31.96 |
| | DON | 144.7 | 1421 |
| HuH-7 | Compound 1 | 1.274 | 9.409 |
| | DON | 5.941 | 13.22 |
| RPMI-7951 | Compound 1 | 0.8937 | 1.286 |
| | DON | 3.47 | 0.9431 |
| HGC-27 | Compound 1 | 13.29 | 0.8468 |
| | DON | 10.04 | 0.5657 |
| BT-474 | Compound 1 | 9.804 | 3.648 |
| | DON | 15.09 | 3.083 |
| BT-549 | Compound 1 | 30.75 | 1.523 |
| | DON | 5.821 | 1.269 |
| OVCAR-3 | Compound 1 | 34.13 | 0.9346 |
| | DON | 30.99 | 0.8323 |
| NCI-H226 | Compound 1 | 14.46 | 15.2 |
| | DON | 1.872 | 12.51 |
| MKN-45 | Compound 1 | 1.654 | 7.023 |
| | DON | 7.524 | 3.661 |
| T84 | Compound 1 | 125.4 | 20.41 |
| | DON | 100.2 | 16.75 |
| NCI-H358 | Compound 1 | 9.605 | 1.899 |
| | DON | 0.9737 | 1.728 |
| HCC1937 | Compound 1 | 155.7 | 136.5 |
| | DON | 156.1 | 25.99 |
| MDA-MB-361 | Compound 1 | 117.2 | 10.72 |
| | DON | 235.4 | 14.93 |
| MDA-MB-435S | Compound 1 | 45.36 | 1.782 |
| | DON | 64.4 | 1.762 |
| SH-SY5Y | Compound 1 | 9.104 | 3.134 |
| | DON | 4.705 | 4.991 |
| HL60 | Compound 1 | 2.026 | 0.4559 |
| | DON | 3.607 | 0.4583 |
| JURKAT | Compound 1 | 0.6559 | 0.2306 |
| | DON | 0.3684 | 0.2337 |
| Daudi | Compound 1 | 1.075 | 0.3949 |
| | DON | 0.3878 | 0.2934 |
| KARPAS-299 | Compound 1 | 0.6359 | 0.4043 |
| | DON | 0.3087 | 0.5913 |
| NAMALWA | Compound 1 | 1.055 | 0.2205 |
| | DON | 0.3332 | 0.3782 |
| U266B1 | Compound 1 | 0.4505 | 0.3977 |
| | DON | 0.4129 | 0.6128 |
| U-937 | Compound 1 | 0.8108 | 0.146 |
| | DON | 0.5661 | 0.1984 |
| Raji | Compound 1 | 3.134 | 0.3979 |
| | DON | 2.039 | 0.421 |

### Experiment 2

### Study Design

Ninety four (94) cell lines were seeded at a density varying from 500 to 7000 cells/well depending on their growth characteristics. PBMC were seeded at the density 50,000-100,000 cells/well. Cells were incubated for 48 h prior to compound treatment. Compound 1 and DON were evaluated at a concertation range of 1 nM up to 100 μM. Compound 1 dilutions in DMSO and DON dilutions in PBS were performed in 96-well 0.5 mL plates (Greiner Bio-One, Germany). Compounds were then diluted 1:100 in RPMI medium. Ninety L of cells were treated by mixing with 10 μL of the compound-containing media (resulting in a final DMSO concentration of 0.1%). The cells were allowed to grow at 37° C. for 120 h. Cells were fixed to the surface by addition of 10% TCA (for adherent growing cells) or 50% TCA (for semi-adherent growing cells or cells growing in suspension). After an hour of incubation at 4° C., plates were washed twice with 400 μL of deionized water and dried. Cells were then stained with 100 μL of 0.04% wt/v SRB. The plates were incubated at room temperature for at least 30 min and washed six times with 1% acetic acid to remove unbound stain. The plates were left to dry at room temperature and bound SRB was solubilized with 100 μL of 10 mM Tris base. Optical density was measured at 492, 520 and 560 nm using a Deelux-LED96 plate reader (Deelux Labortechnik GmbH, Germany).

Cell lines were purchased directly from the ATCC, NCI, CLS, and DSMZ cell line collections. A master bank and working aliquots were prepared. Cells used for the study had undergone less than 20 passages. To rule out potential contamination or wrong assignment, all cell lines were tested by STR analysis. Absence of mycoplasma contamination was confirmed for all cell lines used in the studies.

The cell lines were grown in the media recommended by the suppliers in the presence of 100 U/mL penicillin and 100 μg/mL streptomycin supplied with 10% FCS (PAA, Germany). RPMI 1640, DMEM, and MEM Earle's medium were from PAA (Coelbe, Germany), supplements 2 mM L-glutamine, 1 mM Na-pyruvate and 1% NEAA were from PAA (Coelbe, Germany), 2.5% horse serum, hydrocortisone, transferin, beta-estradiol, selenite and 1 U/mL insulin from Sigma-Aldrich (Munich, Germany). RPMI medium was used for culturing the following cell lines: 5637, 22RV1, 7860, A2780, A431, A549, ACHN, ASPC1, BT20, BXPC3, CAKI1, CLS439, COLO205, COLO678, DLD1, DU145, EFO21, EJ28, HCT15, HS578T, IGROV1, JAR, LOVO, MCF7, MDA MB231, MDA MB435, MDA MB436, MDA MB468, MHHES1, MT3, NCI H292, NCI H358M, NCI H460, NCIH82, OVCAR3, OVCAR4, PANC 1005 (addition of insulin), PBMC, PC3, RDES, SF268, SF295, SKBR3, SK MEL28, SKMEL5, SKOV3, SW620, U2OS, UMUC3, and U031.

DMEM medium was used to culture A204, A375, A673, C33A, CASKI, HCT116, HEPG2, HS729, HT29, J82, MG63, MIAPACA2 (addition of horse serum), PANC1, PLCPRF5, RD, SAOS2, SKLMS1, SKNAS, SNB75, T24, and TE671.

MEM Earle's medium was used for CACO2, CALU6, HEK293, HELA, HT1080, IMR90, JEG3, JIMT1, SKHEP1, SKNSH, and U87MG. Cell seeding conditions were optimized for each cell line. The cell seeding density varied from 500 to 7000 cells/well depending on their growth characteristics. Peripheral blood mononuclear cells (PBMC) were freshly isolated from whole blood of an anonymous donor through density gradient centrifugation using Ficoll solution (d=1.077). In short, blood was diluted 1:1 with PBS and carefully placed on the Ficoll. After centrifugation 1000×g for 15 min (braking rate 0), the ring containing mainly leucocytes was collected in PBS followed by three times washing with PBS to reduce the platelets. PBMC were seeded at the density 50,000-100,000 cells/well. Cells were grown in 5% CO₂ atmosphere in a NB-203XXL incubator (N-BIOTEK Inc., Korea).

### Cell Viability Assay

Ninety-four cell lines were tested in parallel. Cell growth and treatment were performed in 96 well microtitre plates CELLSTAR® (Greiner Bio-One, Germany). Cells harvested from exponential phase cultures by trypsinisation or by splitting (in the case of suspension growing cells) were plated in 90 μL of media at optimal seeding densities. The optimal seeding density for each cell line was determined to ensure exponential growth for the duration of the experiment. All cells growing without anticancer agents were sub-confluent by the end of the treatment, as determined by visual inspection. Cells were allowed to stay for another 48 hours prior to compound treatment. Compound dilutions in DMSO were performed in 96-well 0.5 mL MTP plates (Greiner Bio-One, Germany). Compounds were then diluted 1:100 in RPMI medium. Combined treatment has been performed simultaneously. Ninety μL of cells were treated by mixing with 10 μL of the compound-containing media (resulting in a final DMSO concentration of 0.1%). The cells were allowed to grow at 37° C. for 72 h. In addition, all experiments contained a few plates with cells that were analyzed immediately after the 48 hours recovery period. These plates contained information about the cell number, T_{z}, at time zero, i.e. before treatment, and served to calculate the cytotoxicity. Cells were fixed to the surface by addition of 10% TCA (for adherent growing cells) or 50% TCA (for semi-adherent growing cells or cells growing in suspension). After an hour of incubation at 4° C., plates were washed twice with 400 μL of deionized water and dried. Cells were then stained with 100 μL of 0.04% wt/v SRB. The plates were incubated at room temperature for at least 30 min and washed six times with 1% acetic acid to remove unbound stain. The plates were left to dry at room temperature and bound SRB was solubilized with 100 μL of 10 mM Tris base. Optical density was measured at 492, 520 and 560 nm using a Deelux-LED96 plate reader (Deelux Labortechnik GmbH, Germany).

### Evaluation

The first step in data processing was calculating an average background value for each plate, derived from plates and wells containing medium without cells. The average background optical density was then subtracted from the appropriate control values (containing cells without addition of a drug), from values representing the cells treated with an anticancer agent, and from values of wells containing cells at time zero. Thus the following values were obtained for each experiment: control cell growth, C; cells in the presence of an anticancer agent Tᵢ and cells prior to compound treatment at time zero, T_{z} (or T₀, in some publications).

### Dose Response Curves

The non-linear curve fitting calculations were performed using algorithms and visualization tools developed in-house (Oncolead). The calculations included the dose response curves with the best approximation line, a 95% confidence interval for the 50% effect (see below). IC₅₀ and IC₉₀. One common way to express the effect of an anticancer agent is to measure cell viability and survival in the presence of the test agent as % T/C×100. The relationship between viability and dose is called a dose response curve. Two major values are used to describe this relationship without needing to show the curve: the concentration of test agents giving a % T/C value of 50%, or 50% growth inhibition (IC₅₀), and a % T/C value of 10%, or 90% growth inhibition (IC₉₀). GI₅₀ and TGI and LC₅₀.

Using these measurements, cellular responses can be calculated for incomplete inhibition of cell growth (GI), complete inhibition of cell growth (TGI) and net loss of cells (LC) due to compound activity. Growth inhibition of 50% (GI₅₀) is calculated as 100×[(Tᵢ≠T_{z})/(C≠T_{z})]=50. This is the drug concentration causing a 50% reduction compared to the net protein increase in control cells during the drug incubation period. In other words, GI₅₀ is IC₅₀ corrected for time zero. Similar to IC₉₀, calculated GI₉₀ values are also reported for all compounds tested. TGI was calculated from Tᵢ=T_{z}. LC₅₀, is the concentration of drug causing a 50% reduction in the measured protein at the end of the drug incubation period compared to that at the beginning. It was calculated as 100×[(Tᵢ≠T_{z})/T_{z}]≠50. Low cell seeding density was required due to longer 72 h treatment, and LC₅₀ could rarely be achieved.

### Data Analysis

The IC₅₀ and IC₉₀ values were computed automatically. Visual analysis of all dose response curves was performed to check the quality of the fitting algorithm. In cases where the effect was not reached or exceeded, the values were either approximated or expressed as “-”. All values that were greater than the maximum tested drug concentration were either excluded from the analysis or approximations of IC₁₀ and GI₁₀ were used for analysis. All values were log 10-transformed for analysis. This transformation ensures better data fitting to the normal distribution, a prerequisite to apply any statistical tool. Statistical analyses were performed using proprietary software developed at Oncolead integrated as a database analysis tool. However, except for database comparison, the analysis can be reproduced using either MS Excel or STATISTICA® (StatSoft, Hamburg). Using MS Excel: identification of mean, e.g. mean IC₅₀ (function: “Average”); calculation of,”, delta (GI₅₀−mean GI₅₀); and Z-score (function “Standardize”). Comparison of the activity profiles was performed using Pearson and Spearman correlations. The GI₅₀ and IC₅₀ values for Compound 1 and DON are shown in Table 7.2.

TABLE 7.2

| | | Compound 1 | | DON | |
|---|---|---|---|---|---|
| | | GI₅₀ | IC₅₀ | GI₅₀ | IC₅₀ |
| Cell line | Origin | (μM) | (μM) | (μM) | (μM) |
| 22RV1 | prostate | 1.018 | 1.099 | 0.981 | 1.043 |
| 5637 | bladder | 2.153 | 2.426 | 0.796 | 0.881 |
| 786O | kidney | 3.337 | 3.842 | 2.390 | 2.925 |
| A204 | muscle | 2.648 | 20.302 | 4.989 | 16.965 |
| A2780 | ovary | 3.037 | 3.282 | 3.647 | 3.902 |
| A375 | skin | 4.135 | 4.391 | 3.150 | 3.334 |
| A431 | skin | 14.235 | 18.441 | 6.505 | 8.518 |
| A549 | lung | 9.040 | 9.277 | 4.052 | 4.493 |
| A673 | muscle | 3.488 | 3.865 | 1.948 | 2.198 |
| ACHN | kidney | 7.876 | 8.982 | 5.343 | 6.337 |
| ASPC1 | pancreas | 5.961 | 8.356 | 3.922 | 5.575 |
| BT20 | breast | 19.955 | 53.114 | 16.750 | 51.093 |
| BXPC3 | pancreas | 4.237 | — | 1.454 | — |
| C33A | endometrial | 7.465 | 7.691 | 6.481 | 7.043 |
| CACO2 | colon | 19.372 | 28.199 | 11.177 | 18.614 |
| CAKI1 | kidney | 5.304 | 6.838 | 1.157 | 1.623 |
| CALU6 | lung | 5.884 | 7.074 | 6.054 | 6.967 |
| CASKI | endometrial | 5.903 | 7.835 | 5.489 | 6.512 |
| CLS439 | bladder | 7.568 | 8.426 | 6.870 | 7.820 |
| COLO205 | colon | 2.734 | 4.658 | 1.319 | 2.010 |
| COLO678 | colon | 92.965 | — | 58.890 | 152.295 |
| DLD1 | colon | 9.247 | 9.593 | 0.906 | 1.020 |
| DU145 | prostate | 8.816 | 10.866 | 5.235 | 6.159 |
| EFO21 | ovary | 6.861 | 14.133 | 6.319 | 14.570 |
| EJ28 | bladder | 5.819 | 6.554 | 4.188 | 4.894 |
| GRANTA-519 | hematological | 0.687 | 0.768 | 0.779 | 0.812 |
| HCT116 | colon | 1.313 | 1.335 | 2.055 | 2.100 |
| HCT15 | colon | 20.135 | 21.733 | 1.132 | 1.303 |
| HEK293 | kidney | 9.212 | 10.417 | 10.564 | 15.152 |
| HELA | endometrial | 11.811 | 13.372 | 8.167 | 9.135 |
| HEPG2 | liver | 7.874 | 9.851 | 7.305 | 8.974 |
| HL-60 | hematological | 1.095 | 1.104 | 1.565 | 1.601 |
| HS578T | breast | 36.439 | — | 25.115 | — |
| HS729 | muscle | 10.120 | — | 7.419 | — |
| HT1080 | connective tissue | 2.608 | 3.043 | 0.666 | 0.795 |
| HT29 | colon | 0.880 | 1.068 | 2.892 | 3.405 |
| IGROV1 | ovary | 8.976 | 10.714 | 6.991 | 10.137 |
| IMR90 | lung | 14.563 | — | 11.784 | — |
| J82 | bladder | 14.217 | 15.864 | 23.184 | 28.932 |
| JAR | placenta | 2.235 | 2.409 | 2.815 | 3.038 |
| JEG3 | placenta | 3.868 | 4.784 | 5.785 | 6.638 |
| JIMT1 | breast | 19.450 | 40.564 | 11.186 | 26.787 |
| K-562 | hematological | 1.151 | 1.203 | 1.061 | 1.171 |
| KASUMI-1 | hematological | 3.169 | 7.953 | 5.172 | 12.679 |
| L-363 | hematological | 0.546 | 0.717 | 0.343 | 0.522 |
| LOVO | colon | 5.161 | 5.555 | 4.360 | 4.658 |
| MCF7 | breast | 4.127 | 8.587 | 2.086 | 6.071 |
| MDAMB231 | breast | 11.560 | 14.597 | 8.368 | 13.753 |
| MDAMB435 | skin | 14.738 | 15.201 | 2.476 | 2.658 |
| MDAMB436 | breast | 49.336 | 130.792 | 13.282 | 19.835 |
| MDAMB468 | breast | 0.531 | 0.789 | 0.427 | 0.599 |
| MG63 | bone | 4.597 | 5.375 | 4.609 | 5.305 |
| MHHES1 | bone | 6.188 | 6.337 | 1.851 | 1.995 |
| MIAPACA2 | pancreas | 3.285 | 3.564 | 3.321 | 3.576 |
| MINO | hematological | 0.610 | 0.798 | 0.497 | 0.633 |
| MT3 | breast | 1.105 | 1.462 | 0.638 | 0.791 |
| MV4-11 | hematological | 0.976 | 1.029 | 1.215 | 1.402 |
| NCIH292 | lung | 14.535 | — | 5.717 | 53.103 |
| NCIH358M | lung | 3.312 | 8.924 | 1.609 | 4.984 |
| NCIH460 | lung | 4.742 | 5.008 | 4.106 | 4.311 |
| OVCAR3 | ovary | 1.650 | 4.063 | 0.889 | 2.297 |
| OVCAR4 | ovary | 0.812 | 1.674 | 0.651 | 0.752 |
| PANC1 | pancreas | 7.983 | 8.457 | 12.315 | 13.173 |
| PANC1005 | pancreas | 14.830 | 28.909 | 4.167 | 8.603 |
| PC3 | prostate | 11.536 | 17.928 | 8.373 | 11.742 |
| PLCPRF5 | liver | 6.151 | 7.449 | 6.532 | 8.041 |
| RAMOS | hematological | 6.628 | — | 0.672 | — |
| RD | muscle | 4.051 | 5.444 | 6.795 | 9.129 |
| RDES | bone | 3.951 | 5.481 | 1.959 | 3.176 |
| SAOS2 | bone | 2.797 | 20.785 | 6.360 | 33.168 |
| SF268 | brain | 7.372 | 9.534 | 6.315 | 7.434 |
| SF295 | brain | 9.734 | 10.902 | 4.712 | 5.231 |
| SKBR3 | breast | 2.645 | 3.913 | 2.142 | 3.213 |
| SKHEP1 | liver | 3.925 | 5.124 | 1.794 | 2.620 |
| SKLMS1 | uterus | 1.339 | 2.153 | 1.658 | 2.303 |
| SKMEL28 | skin | 1.229 | 1.608 | 1.687 | 2.191 |
| SKMEL5 | skin | 2.052 | 2.898 | 2.187 | 3.019 |
| SKNAS | brain | 7.624 | 18.581 | 12.635 | — |
| SKNSH | brain | 8.180 | 9.151 | 5.036 | 7.105 |
| SKOV3 | ovary | 1.255 | 3.805 | 0.769 | 1.214 |
| SNB75 | brain | 18.405 | 40.018 | 15.547 | — |
| SU-DHL-10 | hematological | 4.180 | — | 8.417 | — |
| SU-DHL-6 | hematological | 0.971 | 1.134 | 0.623 | 0.761 |
| SW620 | colon | 7.957 | 8.583 | 5.376 | 6.245 |
| T24 | bladder | 4.713 | 4.953 | 3.859 | 4.004 |
| TE671 | muscle | 5.439 | 5.967 | 4.633 | 5.102 |
| THP-1 | hematological | 1.990 | 2.255 | 1.017 | 1.088 |
| U2OS | bone | 10.011 | 12.931 | 5.907 | 8.404 |
| U87MG | brain | 2.376 | 4.098 | 10.531 | 11.293 |
| UMUC3 | bladder | 2.317 | 2.752 | 2.233 | 2.758 |
| UO31 | kidney | 17.689 | 44.878 | 0.981 | 18.647 |
| WSU-NHL | hematological | 1.158 | 1.504 | 0.796 | 0.639 |

### Example 8

### In Vivo Anti-Tumor Efficacy of Compound 1 by IV and SC Routes in the CT26.WT Model

Female BALB/c mice were inoculated subcutaneously in the right flank with CT26.WT cells for tumor development. Six days after tumor inoculation, mice with tumor sizes ranging from 32-77 mm³ (average tumor size 56 mm³) were selected and assigned into 6 groups using stratified randomization with 8 mice per group based upon their tumor volumes. The treatments were started from the day of randomization (defined as D0), Group 1 was treated with Vehicle control, q.d.×5 days on (Mon-Fri)×4 cycles s.c.; Group 2 was treated with Compound 1, 0.7 mpk×5 days on (Mon-Fri)×4 cycles s.c.; Group 3 was treated with Compound 1, 1.4 mpk×5 days on (Mon-Fri)×4 cycles s.c.; Group 4 was treated with Compound 1, 4.5 mpk×3-days on (Mon, Wed, Fri)×4 cycles, iv; Group 5 was treated with Compound 1, 7 mpk×2-days on (Mon,Thu)×4 cycles, iv; Group 6 was treated with Compound 1, 14 mpk×1-day on (Mon)×4 cycles, iv. The tumor sizes were measured three times per week during the treatment. The entire study was terminated on D44 after start of the treatment. All treatments were well-tolerated without any adverse effects or significant body weight loss.

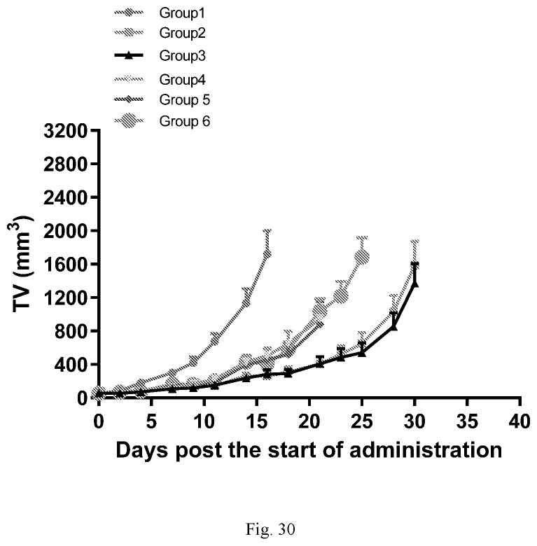

Mean tumor volume over time in female BALB/c mice bearing CT26.WT tumors dosed with Compound 1 is shown in Table 8.1. The tumor growth inhibition analysis is shown in Table 8.2. The survival analysis is shown in Table 8.3. The tumor growth curves are shown in FIG. 30.

TABLE 8.1

| | Tumor volume (mm³)^{a} | | | | | |
|---|---|---|---|---|---|---|
| Days | Group 1 | Group 2 | Group 3 | Group 4 | Group 5 | Group 6 |
| 0 | 58 ± 5 | 56 ± 4 | 56 ± 5 | 56 ± 3 | 56 ± 4 | 56 ± 5 |
| 2 | 80 ± 7 | 62 ± 7 | 55 ± 7 | 70 ± 6 | 71 ± 9 | 69 ± 7 |
| 4 | 181 ± 21 | 89 ± 10 | 70 ± 8 | 92 ± 11 | 78 ± 11 | 78 ± 5 |
| 7 | 299 ± 34 | 124 ± 20 | 107 ± 19 | 126 ± 16 | 142 ± 26 | 163 ± 24 |
| 9 | 421 ± 67 | 140 ± 22 | 118 ± 21 | 168 ± 24 | 166 ± 37 | 160 ± 22 |
| 11 | 672 ± 94 | 143 ± 21 | 150 ± 26 | 223 ± 40 | 185 ± 45 | 204 ± 29 |
| 14 | 1132 ± 174 | 261 ± 45 | 235 ± 35 | 427 ± 73 | 395 ± 114 | 425 ± 72 |
| 16 | 1718 ± 282 | 259 ± 52 | 282 ± 52 | 503 ± 94 | 455 ± 131 | 406 ± 74 |
| 18 | | 308 ± 63 | 292 ± 49 | 648 ± 148 | 518 ± 135 | 578 ± 89 |
| 21 | | 407 ± 83 | 406 ± 85 | 948 ± 230 | 878 ± 272 | 1035 ± 150 |
| 23 | | 522 ± 102 | 484 ± 103 | | | 1216 ± 177 |
| 25 | | 648 ± 130 | 539 ± 119 | | | 1684 ± 231 |
| 28 | | 1030 ± 194 | 853 ± 163 | | | |
| 30 | | 1585 ± 288 | 1372 ± 230 | | | |

^{a}Mean ± SEM; n = 8; Group 1: Vehicle control, q.d. x 5 days on (Mon-Fri) x 4 cycles s.c.; Group 2: Compound 1, 0.7 mpk x 5 days on (Mon-Fri) x 4 cycles s.c.; Group 3: Compound 1, 1.4 mpk x 5 days on (Mon-Fri) x 4 cycles s.c.; Group 4: Compound 1, 4.5 mpk x 3-days on (Mon, Wed, Fri) x 4 cycles, iv; Group 5: Compound 1, 7 mpk x 2-days on (Mon, Thu) x 4 cycles, iv; Group 6: Compound 1, 14 mpk x 1-day on (Mon) x 4 cycles, iv

TABLE 8.2

| | Tumor Size | | | | |
|---|---|---|---|---|---|
| | (mm³) | RTV | T/C | TGI | |
| Treatment | at day 16 | at day 16 | (%) | (%) | p value |
| Group1 | 1718 ± 282 | 31.22 ± 3.93 | — | — | — |
| Group2 | 259 ± 52 | 4.45 ± 0.74 | 14.25 | 87.81 | <0.001 |
| Group3 | 282 ± 52 | 4.94 ± 0.82 | 15.82 | 86.45 | <0.001 |
| Group4 | 503 ± 94 | 8.92 ± 1.58 | 28.57 | 73.09 | <0.001 |
| Group5 | 455 ± 131 | 7.58 ± 1.97 | 24.28 | 76.00 | <0.001 |
| Group6 | 406 ± 74 | 7.02 ± 0.87 | 22.49 | 78.92 | <0.001 |

Mean ± SEM.
All groups compare to Group 1

TABLE 8.3

| | MST | ILS | | | |
|---|---|---|---|---|---|
| Treatment | (day) | (%) | P^{a} value | P^{b} value | P^{c} value |
| Group1 | 18 | — | — | | |
| Group2 | 33.5 | 86.1 | <0.0001 | | |
| Group3 | 33.5 | 86.1 | <0.0001 | | |
| Group4 | 29 | 61.1 | <0.001 | <0.05 | <0.01 |
| Group5 | 29 | 61.1 | <0.001 | ns | ns |
| Group6 | 26.5 | 47.2 | <0.0001 | <0.01 | <0.01 |

^{a}All groups compare to G1;
^{b}Groups 4-6 compare to G2;
^{c}Groups 4-6 compare to G3

### Example 9

### In Vivo Anti-Tumor Efficacy of Compound 1 and Compound 3 by IV and SC Routes in the MC38 Model

Female C57BL/6 mice were inoculated subcutaneously in the right flank with MC38 cells for tumor development. Six days after tumor inoculation, mice with tumor sizes ranging from 52-97 mm³ (average tumor size 71 mm³) were selected and assigned into 10 groups using stratified randomization with 8 mice per group based upon their tumor volumes. The treatments were started from the day of randomization (defined as D0), Group 1 was treated with Vehicle control, SC qd×5 days on (Mon-Fri)×4 cycles; Group 2 was treated with Compound 1, 1.4 mpk SC, qd×5 days on (Mon-Fri)×4 cycles; Group 3 was treated with Compound 1, 7 mpk IV, biw (Mon, Thu)×4 cycles; Group 4 was treated with Compound 1, 14 mpk IV, biw (Mon, Thu)×4 cycles; Group 5 was treated with Compound 1, 14 mpk IV, qw (Mon)×4 cycles; Group 6 was treated with Compound 1, 21 mpk IV, qw (Mon)×4 cycles; Group 7 was treated with Compound 3, 1 mpk SC, qd 5 days on (Mon-Fri)×4 cycles; Group 8 was treated with Compound 3, 3 mpk SC, qd 5 days on (Mon-Fri)×4 cycles; Group 9 was treated with Compound 3, 9 mpk SC, qd 5 days on (Mon-Fri)×4 cycles; and Group 10 was treated with Compound 3, 7 mpk IV, tiw (Mon, Wed, Fri)×4 cycles. The tumor sizes were measured three times per week during the treatment. Survival was monitored with tumor volume exceeding 2000 mm³ as endpoint. The entire study was terminated on D44 after start of the treatment. All treatments were well-tolerated without any adverse effects or significant body weight loss.

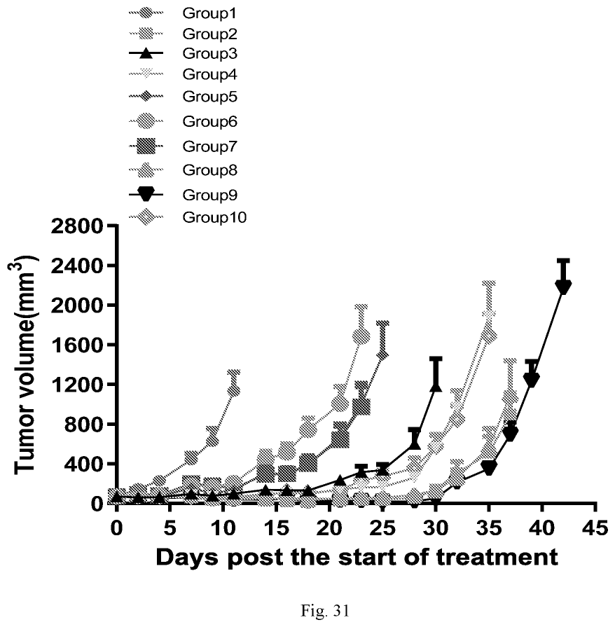

Mean tumor volume over time in female BALB/c mice bearing MC38 tumors dosed with Compound 1 or Compound 3 is shown in Table 9.1. The tumor growth inhibition analysis is shown in Table 9.2. The survival analysis is shown in Table 9.3. The tumor growth curves are shown in FIG. 31.

TABLE 9.1

| | Tumor volume (mm³)^{a} | | | | | | | | | |
|---|---|---|---|---|---|---|---|---|---|---|
| Day | Group 1 | Group 2 | Group 3 | Group 4 | Group 5 | Group 6 | Group 7 | Group 8 | Group 9 | Group 10 |
| 0 | 71 ± 4 | 71 ± 5 | 71 ± 4 | 72 ± 6 | 70 ± 4 | 71 ± 4 | 70 ± 4 | 70 ± 5 | 71 ± 5 | 71 ± 5 |
| 2 | 148 ± 15 | 64 ± 5 | 61 ± 9 | 80 ± 12 | 67 ± 5 | 79 ± 10 | 91 ± 9 | 88 ± 7 | 88 ± 7 | 86 ± 5 |
| 4 | 229 ± 36 | 68 ± 10 | 64 ± 7 | 70 ± 8 | 77 ± 12 | 80 ± 9 | 83 ± 9 | 59 ± 9 | 59 ± 9 | 76 ± 7 |
| 7 | 443 ± 65 | 61 ± 5 | 98 ± 8 | 86 ± 13 | 152 ± 20 | 182 ± 24 | 201 ± 26 | 57 ± 14 | 57 ± 14 | 76 ± 8 |
| 9 | 628 ± 127 | 39 ± 3 | 79 ± 7 | 70 ± 11 | 105 ± 12 | 164 ± 21 | 181 ± 18 | 45 ± 6 | 45 ± 6 | 61 ± 7 |
| 11 | 1132 ± 192 | 43 ± 6 | 99 ± 11 | 69 ± 11 | 134 ± 21 | 206 ± 33 | 152 ± 10 | 39 ± 5 | 39 ± 5 | 68 ± 13 |
| 14 | | 51 ± 9 | 140 ± 16 | 95 ± 14 | 312 ± 56 | 440 ± 87 | 293 ± 60 | 41 ± 7 | 41 ± 7 | 85 ± 16 |
| 16 | | 50 ± 7 | 134 ± 10 | 107 ± 25 | | 515 ± 84 | 298 ± 60 | 47 ± 7 | 47 ± 7 | 115 ± 21 |
| 18 | | 52 ± 9 | 133 ± 15 | 96 ± 22 | | 740 ± 120 | 400 ± 79 | 22 ± 4 | 22 ± 4 | 107 ± 21 |
| 21 | | 44 ± 7 | 236 ± 36 | 144 ± 45 | | 1005 ± 174 | 644 ± 160 | 29 ± 3 | 29 ± 3 | 131 ± 21 |
| 23 | | 41 ± 9 | 317 ± 59 | 163 ± 41 | | 1683 ± 302 | 975 ± 238 | 26 ± 2 | 26 ± 2 | 233 ± 46 |
| 25 | | 42 ± 11 | 337 ± 56 | 167 ± 47 | | | | 23 ± 4 | 23 ± 4 | 273 ± 59 |
| 28 | | 55 ± 14 | 602 ± 144 | 261 ± 54 | | | | 22 ± 4 | 22 ± 4 | 356 ± 103 |
| 30 | | 140 ± 50 | 1181 ± 278 | 562 ± 124 | | | | 52 ± 8 | 52 ± 8 | 575 ± 121 |
| 32 | | 303 ± 119 | | 967 ± 171 | | | | 213 ± 47 | 213 ± 47 | 849 ± 134 |
| 35 | | 509 ± 166 | | 1884 ± 337 | | | | 351 ± 61 | 351 ± 61 | 1688 ± 223 |
| 37 | | 896 ± 217 | | | | | | 692 ± 129 | 692 ± 129 | |
| 39 | | | | | | | | | 1235 ± 196 | |
| 42 | | | | | | | | | 2174 ± 275 | |

^{a}Mean ± SEM; n = 8; Group 1: Vehicle control, SC qd x5 days on (Mon-Fri) x 4 cycles; Group 2: Compound 1, 1.4 mpk SC, qd 5 days on (Mon-Fri) x 4 cycles; Group 3: Compound 1, 7 mpk IV, biw (Mon, Thu) x 4 cycles; Group 4: Compound 1, 14 mpk IV, biw (Mon, Thu) x 4 cycles; Group 5: Compound 1, 14 mpk IV, qw (Mon) x 4 cycles; Group 6: Compound 1, 21 mpk IV, qw (Mon) x 4 cycles iv; Group 7: Compound 3, 1 mpk SC, qd 5 days on (Mon-Fri) x 4 cycles; Group 8: Compound 3, 3 mpk SC, qd 5 days on (Mon-Fri) x 4 cycles; Group 9: Compound 3, 9 mpk SC, qd 5 days on (Mon-Fri) x 4 cycles; Group 10: Compound 3, 7 mpk IV, tiw (Mon, Wed, Fri) x 4 cycles

TABLE 9.2

| | Tumor Size | | | | |
|---|---|---|---|---|---|
| | (mm³) | RTV | T/C | TGI | |
| Treatment | at day 11 | at day 11 | (%) | (%) | p value |
| Group 1 | 1132 ± 192 | 16.25 ± 2.73 | — | — | — |
| Group 2 | 43 ± 6 | 0.63 ± 0.11 | 3.86 | 102.69 | <0.0001 |
| Group 3 | 99 ± 11 | 1.44 ± 0.20 | 8.87 | 97.42 | <0.0001 |
| Group 4 | 69 ± 11 | 1.06 ± 0.22 | 6.51 | 100.28 | <0.0001 |
| Group 5 | 134 ± 21 | 2.06 ± 0.40 | 12.69 | 93.98 | <0.0001 |
| Group 6 | 206 ± 33 | 2.86 ± 0.39 | 17.57 | 87.26 | <0.0001 |
| Group 7 | 152 ± 10 | 2.22 ± 0.22 | 13.66 | 92.34 | <0.0001 |
| Group 8 | 39 ± 5 | 0.85 ± 0.11 | 5.22 | 101.21 | <0.0001 |
| Group 9 | 39 ± 5 | 0.56 ± 0.07 | 3.45 | 102.98 | <0.0001 |

Mean ± SEM;
n = 8

TABLE 9.3

| | MST | ILS | |
|---|---|---|---|
| Treatment | (day) | (%) | p^{a} value |
| Group 1 | 15 | — | — |
| Group 2 | 40.5 | 170.0 | <0.0001 |
| Group 3 | 33.5 | 123.3 | <0.0001 |
| Group 4 | 35 | 133.3 | <0.0001 |
| Group 5 | 28 | 86.7 | <0.0001 |
| Group 6 | 24 | 60.0 | <0.0001 |
| Group 7 | 31 | 106.7 | <0.0001 |
| Group 8 | 40.5 | 170.0 | <0.0001 |
| Group 9 | 42 | 180.0 | <0.0001 |
| Group 10 | 37 | 146.7 | <0.001 |

^{a}All groups compare to Group 1

### Example 10

### In Vivo Anti-Tumor Efficacy of Compound 1 and Compound 1 in Combination with Anti-PD-1 in the CT26.WT Syngeneic Model

Female BALB/c mice were inoculated subcutaneously in the right flank with CT26.WT cells for tumor development. Six days after tumor inoculation, mice with tumor sizes ranging from 30-81 mm³ (average tumor size 55 mm³) were selected and assigned into 10 groups using stratified randomization with 8 mice per group based upon their tumor volumes. The treatments were started from the day of randomization (defined as D0), Group 1 was treated with Vehicle control, 10 mpk, SC, qd 5 days on (Mon-Fri)×4 cycles; Group 2 was treated with Anti-PD-1, 10 mpk, IP, q4d×6; Group 3 was treated with Compound 1, 1.4 mpk, SC, qd 5 days on (Mon-Fri)×4 cycles; Group 4 was treated with Compound 1+anti-PD-1, 1.4 mpk+10 mpk, SC, qd 5 days on (Mon-Fri)×4 cycles+IP, q4d×6; Group 5 was treated with Compound 1, 10.5 mpk, IV, biw (Mon, Thu)×4 cycles; Group 6 was treated with Compound 1+anti-PD-1, 10.5 mpk+10 mpk, IV, biw (Mon, Thu)×4 cycles+IP, q4d×6; Group 7 was treated with Compound 1, 7 mpk IV, tiw (Mon, Wed, Fri)×4 cycles; Group 8 was treated with Compound 1+anti-PD-1, 7 mpk+10 mpk, IV, tiw (Mon, Wed, Fri)×4 cycles+IP, q4d×6; Group 9 was treated with Compound 1, 7 mpk IV, tiw (Mon, Tue, Wed)×4 cycles; and Group 10 was treated with Compound 1+anti-PD-1, 7 mpk+10 mpk, IV, tiw (Mon, Tue, Wed)×4 cycles+IP, q4d×6. The tumor sizes were measured three times per week during the treatment. Survival was monitored with tumor volume exceeding 2000 mm³ as endpoint. The entire study was terminated on D56 after start of the treatment. All treatments were well-tolerated without any adverse effects or significant body weight loss.

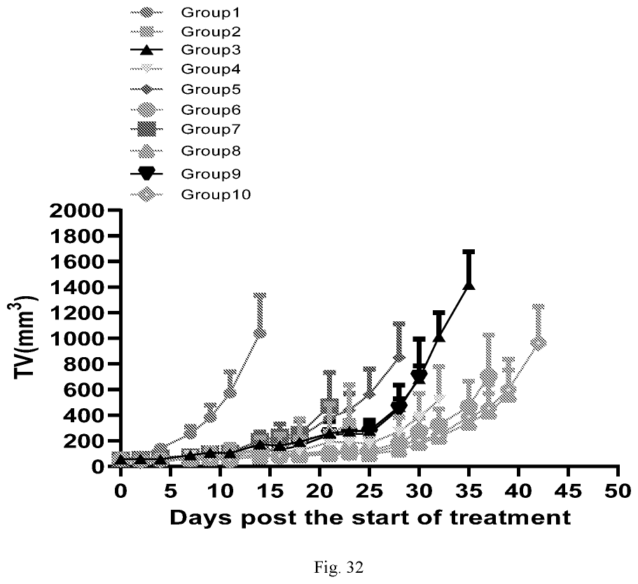

Mean tumor volume over time in female BALB/c mice bearing CT26.WT tumors dosed with Compound 1 or Compound 1 in combination with anti-PD-1 is shown in Table 10.1. The tumor growth inhibition analysis is shown in Table 10.2. The survival analysis is shown in Table 10.3. The tumor growth curves are shown in FIG. 32.

TABLE 10.1

| | Tumor volume (mm³)^{a} | | | | | | | | | |
|---|---|---|---|---|---|---|---|---|---|---|
| Day | Group 1 | Group 2 | Group 3 | Group 4 | Group 5 | Group 6 | Group 7 | Group 8 | Group 9 | Group 10 |
| 0 | 56 ± 5 | 54 ± 5 | 56 ± 5 | 56 ± 5 | 56 ± 5 | 55 ± 4 | 55 ± 5 | 55 ± 5 | 56 ± 4 | 55 ± 5 |
| 2 | 82 ± 9 | 46 ± 6 | 59 ± 5 | 63 ± 9 | 52 ± 5 | 52 ± 6 | 61 ± 9 | 53 ± 6 | 40 ± 2 | 42 ± 4 |
| 4 | 144 ± 30 | 59 ± 18 | 55 ± 5 | 60 ± 9 | 56 ± 7 | 52 ± 5 | 74 ± 12 | 60 ± 8 | 48 ± 5 | 49 ± 4 |
| 7 | 252 ± 60 | 65 ± 27 | 89 ± 9 | 85 ± 15 | 71 ± 12 | 54 ± 5 | 81 ± 14 | 56 ± 6 | 53 ± 8 | 63 ± 6 |
| 9 | 381 ± 98 | 99 ± 46 | 108 ± 11 | 73 ± 9 | 92 ± 13 | 61 ± 10 | 106 ± 21 | 63 ± 9 | 58 ± 14 | 65 ± 7 |
| 11 | 573 ± 167 | 107 ± 65 | 103 ± 13 | 85 ± 15 | 113 ± 24 | 49 ± 9 | 111 ± 33 | 61 ± 11 | 79 ± 19 | 74 ± 6 |
| 14 | 1039 ± 296 | 132 ± 93 | 172 ± 29 | 123 ± 28 | 170 ± 51 | 89 ± 28 | 201 ± 67 | 73 ± 17 | 132 ± 46 | 97 ± 11 |
| 16 | | 140 ± 109 | 163 ± 20 | 112 ± 31 | 214 ± 60 | 76 ± 28 | 238 ± 94 | 87 ± 24 | 131 ± 52 | 92 ± 10 |
| 18 | | 203 ± 169 | 191 ± 26 | 124 ± 35 | 226 ± 59 | 87 ± 34 | 256 ± 101 | 84 ± 24 | 160 ± 65 | 100 ± 14 |
| 21 | | 255 ± 230 | 258 ± 25 | 190 ± 59 | 381 ± 119 | 106 ± 41 | 471 ± 261 | 92 ± 24 | 248 ± 104 | 119 ± 17 |
| 23 | | 333 ± 304 | 276 ± 36 | 188 ± 63 | 439 ± 128 | 126 ± 49 | | 107 ± 27 | 252 ± 103 | 104 ± 16 |
| 25 | | | 278 ± 42 | 179 ± 68 | 562 ± 197 | 123 ± 47 | | 94 ± 24 | 255 ± 106 | 103 ± 18 |
| 28 | | | 466 ± 62 | 272 ± 112 | 849 ± 264 | 181 ± 67 | | 128 ± 37 | 447 ± 186 | 159 ± 35 |
| 30 | | | 682 ± 103 | 385 ± 184 | | 275 ± 104 | | 181 ± 63 | 695 ± 300 | 207 ± 48 |
| 32 | | | 1012 ± 189 | 519 ± 260 | | 325 ± 124 | | 229 ± 88 | | 241 ± 56 |
| 35 | | | 1417 ± 260 | | | 469 ± 196 | | 339 ± 156 | | 389 ± 100 |
| 37 | | | | | | 706 ± 319 | | 426 ± 215 | | 454 ± 115 |
| 39 | | | | | | | | 556 ± 280 | | 599 ± 153 |
| 42 | | | | | | | | | | 964 ± 284 |

^{a}Mean ± SEM; n = 8; Group 1: Vehicle control, 10 mpk, SC, qd 5 days on (Mon-Fri) x 4 cycles; Group 2: Anti-PD-1, 10 mpk, IP, q4d x 6; Group 3: Compound 1, 1.4 mpk, SC, qd 5 days on (Mon-Fri) x 4 cycles; Group 4: Compound 1 + anti-PD-1, 1.4 mpk + 10 mpk, SC, qd 5 days on (Mon-Fri) x 4 cycles + IP, q4d x 6; Group 5: Compound 1, 10.5 mpk, IV, biw (Mon, Thu) x 4 cycles; Group 6: Compound 1 + anti-PD-1, 10.5 mpk + 10 mpk, IV, biw (Mon, Thu) x 4 cycles + IP, q4d x 6; Group 7: Compound 1, 7 mpk IV, tiw (Mon, Wed, Fri) x 4 cycles; Group 8: Compound 1 + anti-PD-1, 7 mpk + 10 mpk, IV, tiw (Mon, Wed, Fri) x 4 cycles + IP, q4d x 6; Group 9: Compound 1, 7 mpk IV, tiw (Mon, Tue, Wed) x 4 cycles; Group10: Compound 1 + anti-PD-1, 7 mpk + 10 mpk, IV, tiw (Mon, Tue, Wed) x 4 cycles + IP, q4d x 6

TABLE 10.2

| | Tumor Size | | | | |
|---|---|---|---|---|---|
| | (mm³) | RTV | T/C | TGI | |
| Treatment | at day 14 | at day 14 | (%) | (%) | p value |
| Group 1 | 1039 ± 296 | 18.00 ± 5.03 | — | — | — |
| Group 2 | 132 ± 93 | 2.41 ± 1.75 | 13.39 | 92.12 | <0.0001 |
| Group 3 | 172 ± 29 | 3.07 ± 0.38 | 17.06 | 88.16 | <0.0001 |
| Group 4 | 123 ± 28 | 2.32 ± 0.47 | 12.89 | 93.18 | <0.0001 |
| Group 5 | 170 ± 51 | 3.21 ± 1.03 | 17.83 | 88.39 | <0.0001 |
| Group 6 | 89 ± 28 | 1.88 ± 0.65 | 10.44 | 96.55 | <0.0001 |
| Group 7 | 201 ± 67 | 3.34 ± 0.97 | 18.56 | 85.12 | <0.0001 |
| Group 8 | 73 ± 17 | 1.41 ± 0.32 | 7.83 | 98.16 | <0.0001 |
| Group 9 | 132 ± 46 | 2.32 ± 0.84 | 12.89 | 92.20 | <0.0001 |
| Group 10 | 97 ± 11 | 1.76 ± 0.2 | 9.78 | 95.72 | <0.0001 |

Mean ± SEM;
n = 8

TABLE 10.3

| | MST | ILS | |
|---|---|---|---|
| Treatment | (day) | (%) | P^{a} value |
| Group 1 | 21 | — | — |
| Group 2 | — | — | <0.001 |
| Group 3 | 38 | 81.0 | <0.0001 |
| Group 4 | 47.5 | 126.2 | <0.0001 |
| Group 5 | 37 | 76.2 | <0.001 |
| Group 6 | 52.5 | 150.0 | <0.0001 |
| Group 7 | 37 | 76.2 | <0.001 |
| Group 8 | 53 | 152.4 | <0.0001 |
| Group 9 | 40.5 | 92.9 | <0.0001 |
| Group 10 | 47.5 | 126.2 | <0.0001 |

^{a}All groups compare to Group 1

### Example 11

### In Vivo Anti-Tumor Efficacy of Compound 1 in the A549 Xenograft Model

Female BALB/c nude mice were inoculated subcutaneously in the right flank with A549 cells for tumor development. Eight days after tumor inoculation, mice with tumor sizes ranging from 100-200 mm³ (average tumor size 156 mm³) were selected and assigned into groups using stratified randomization with 8 mice per group based upon their tumor volumes. The treatments were started from the day of randomization (defined as D0), Group 1 was treated with Vehicle control, 10 ml/kg, IP, bid×21; Group 4 was treated with Compound 1; 1.4 mg/kg, SC, 5d ON, 2 d OFF×3 cycles; and Group 5 was treated with Compound 1, 3.3 mg/kg, IV, tiw, (Fri, Sun, Tues), ×3 cycles. The tumor sizes were measured two times per week during the treatment. The entire study was terminated on D21 after start of the treatment. All treatments were well-tolerated without any adverse effects or significant body weight loss.

Mean tumor volume over time in female BALB/c mice bearing A549 tumors dosed with Compound 1 is shown in Table 11.1. The tumor growth inhibition analysis is shown in Table 11.2.

TABLE 11.1

| | Tumor volume (mm³)^{a} | | |
|---|---|---|---|
| Day | Group 1 | Group 4 | Group 5 |
| 0 | 155 ± 11 | 155 ± 10 | 156 ± 10 |
| 4 | 202 ± 12 | 187 ± 12 | 180 ± 12 |
| 7 | 294 ± 14 | 273 ± 25 | 268 ± 21 |
| 11 | 538 ± 74 | 500 ± 59 | 473 ± 39 |
| 14 | 766 ± 71 | 658 ± 92 | 647 ± 66 |
| 18 | 1180 ± 83 | 1050 ± 112 | 1107 ± 119 |
| 21 | 1757 ± 219 | 1300 ± 161 | 1453 ± 171 |

^{a}Mean ± SEM;
n = 8;
Group 1: Vehicle control, 10 ml/kg, IP, bid x 21;
Group 4: Compound 1, 1.4 mg/kg, SC, 5 d ON, 2 d OFF x 3 cycles;
Group 5: Compound 1, 3.3 mg/kg, IV, tiw, (Fri, Sun, Tues) x 3 cycles

TABLE 11.2

| | Tumor Size | | | | |
|---|---|---|---|---|---|
| | (mm³) | RTV | T/C | TGI | |
| Treatment | at day 21 | at day 21 | (%) | (%) | p value |
| Group 1 | 1757 ± 219 | 11.35 ± 1.16 | — | — | — |
| Group 4 | 1300 ± 161 | 8.61 ± 1.30 | 75.88 | 28.57 | 0.0009 |
| Group 5 | 1453 ± 171 | 9.55 ± 1.16 | 84.12 | 19.04 | 0.0539 |

Mean ± SEM;
n = 8

### Example 12

### In Vivo Anti-Tumor Efficacy of Compound 1 in the HCT116 Xenograft Model

Female BALB/c nude mice were inoculated subcutaneously in the right flank with HCT116 cells for tumor development. Nine days after tumor inoculation, mice with tumor sizes ranging from 90-260 mm³ (average tumor size 155 mm³) were selected and assigned into groups using stratified randomization with 8 mice per group based upon their tumor volumes. The treatments were started from the day of randomization (defined as D0), Group 1 was treated with Vehicle control, 10 ml/kg, IP, bid×21; Group 4 was treated with Compound 1, 1.4 mg/kg, SC, 5d ON, 2 d OFF×3 cycles; and Group 5 was treated with Compound 1, 3.3 mg/kg, IV, tiw, (Thu, Sat, Mon)×3 cycles. The tumor sizes were measured two times per week during the treatment. The entire study was terminated on D18 after start of the treatment. All treatments were well-tolerated without any adverse effects or significant body weight loss.

Mean tumor volume over time in female BALB/c mice bearing HCT116 tumors dosed with Compound 1 is shown in Table 12.1. The tumor growth inhibition analysis is shown in Table 12.2.

TABLE 12.1

| | Tumor volume (mm³)^{a} | | |
|---|---|---|---|
| Day | Group 1 | Group 4 | Group 5 |
| 0 | 154 ± 20 | 155 ± 19 | 156 ± 19 |
| 4 | 334 ± 40 | 227 ± 39 | 188 ± 23 |
| 7 | 600 ± 88 | 294 ± 53 | 378 ± 60 |
| 11 | 1125 ± 152 | 602 ± 72 | 571 ± 82 |
| 14 | | 964 ± 176 | 861 ± 134 |
| 18 | | | 1354 ± 204 |

^{a}Mean ± SEM;
n = 8;
Group 1: Vehicle control, 10 ml/kg,IP, bid x 21;
Group 4: Compound 1, 1.4 mg/kg, SC, 5 d ON, 2 d OFF x 3 cycles;
Group 5: Compound 1, 3.3 mg/kg, IV, tiw, (Fri, Sun, Tues) x 3 cycles

TABLE 12.2

| | Tumor Size | | | | |
|---|---|---|---|---|---|
| | (mm³) | RTV | T/C | TGI | |
| Treatment | at day 11 | at day 11 | (%) | (%) | p value |
| Group 1 | 1125 ± 152 | 7.68 ± 0.84 | — | — | — |
| Group 4 | 602 ± 72 | 4.061 ± 0.33 | 52.25 | 53.92 | <0.0001 |
| Group 5 | 571 ± 82 | 3.70 ± 0.45 | 48.17 | 57.23 | <0.0001 |

Mean ± SEM;
n = 8

### Example 13

### In Vivo Anti-Tumor Efficacy of Compound 1 in the LK-2 Xenograft Model

Female BALB/c nude mice were inoculated subcutaneously in the right flank with LK-2 cells for tumor development. Four days after tumor inoculation, mice with tumor sizes reaching approximately 171 mm³ (range from 112 mm³ to 222 mm³) were selected and assigned into groups using stratified randomization with 8 mice per group based upon their tumor volumes. The treatments were started from the day of randomization (defined as D0), Group 1 was treated with Vehicle control, 10 ml/kg, IP, bid×19; Group 2 was treated with Compound 1, 1.4 mg/kg, SC, 5d ON, 2d OFF×2 cycles, followed Compound 1, 2.8 mg/kg, SC, 5d ON, 2 d OFF×1 cycle; Group 3 was treated with Compound 1, 7 mg/kg, IV, tiw (Mon, Wed, Fri)×3 cycles; and Group 4 was treated with Compound 1, 3.3 mg/kg, IV, tiw (Mon, Wed, Fri)×3 cycles. The tumor sizes were measured two times per week during the treatment. The entire study was terminated on D18 after start of the treatment. All treatments were well-tolerated without any adverse effects or significant body weight loss.

Mean tumor volume over time in female BALB/c mice bearing LK-2 tumors dosed with Compound 1 is shown in Table 13.1. The tumor growth inhibition analysis is shown in Table 13.2.

TABLE 13.1

| | Tumor volume (mm³)^{a} | | | |
|---|---|---|---|---|
| Day | Group 1 | Group 2 | Group 3 | Group 4 |
| 0 | 172 ± 10 | 171 ± 10 | 171 ± 11 | 170 ± 10 |
| 2 | 239 ± 20 | 207 ± 19 | 196 ± 9 | 193 ± 6 |
| 4 | 281 ± 26 | 217 ± 24 | 213 ± 8 | 211 ± 6 |
| 7 | 458 ± 54 | 419 ± 81 | 256 ± 13 | 374 ± 47 |
| 10 | 971 ± 112 | 780 ± 128 | 483 ± 44 | 577 ± 68 |
| 14 | 1655 ± 272 | 1359 ± 207 | 1050 ± 113 | 1294 ± 172 |
| 17 | | | 1565 ± 175 | |

^{a}Mean ± SEM;
n = 8;
Group 1: Vehicle control, 10 ml/kg, IP, bid x 21;
Group 2: Compound 1, 1.4 mg/kg, SC, 5 d ON, 2 d OFF x 2 cycles, followed by DRP-104, 2.8 mg/kg, SC, 5 d ON, 2 d OFF x 1 cycle;
Group 3: Compound 1, 7 mg/kg, IV, tiw (Mon, Wed, Fri) x 3 cycles;
Group 4: Compound 1, 3.3 mg/kg, IV, tiw (Mon, Wed, Fri) x 3 cycles

TABLE 13.2

| | Tumor Size | | | | |
|---|---|---|---|---|---|
| | (mm³) | RTV | T/C | TGI | |
| Treatment | at day 14 | at day 11 | (%) | (%) | p value |
| Group 1 | 1655 ± 272 | 9.64 ± 1.48 | — | — | — |
| Group 2 | 1359 ± 207 | 8.13 ± 1.31 | 84.34 | 19.84 | 0.0579 |
| Group 3 | 1050 ± 113 | 6.29 ± 0.75 | 65.30 | 40.73 | <0.0001 |
| Group 4 | 1294 ± 172 | 7.66 ± 0.88 | 79.51 | 24.13 | 0.0136 |

Mean ± SEM;
n = 8

### Example 14

### Synthesis of (S)-2-((S)-2-acetamido-3-(1H-indol-3-yl)propanamido)-6-diazo-5-oxohexanoic acid Compound 3

Compound 3 can be prepared as shown in Scheme 1.

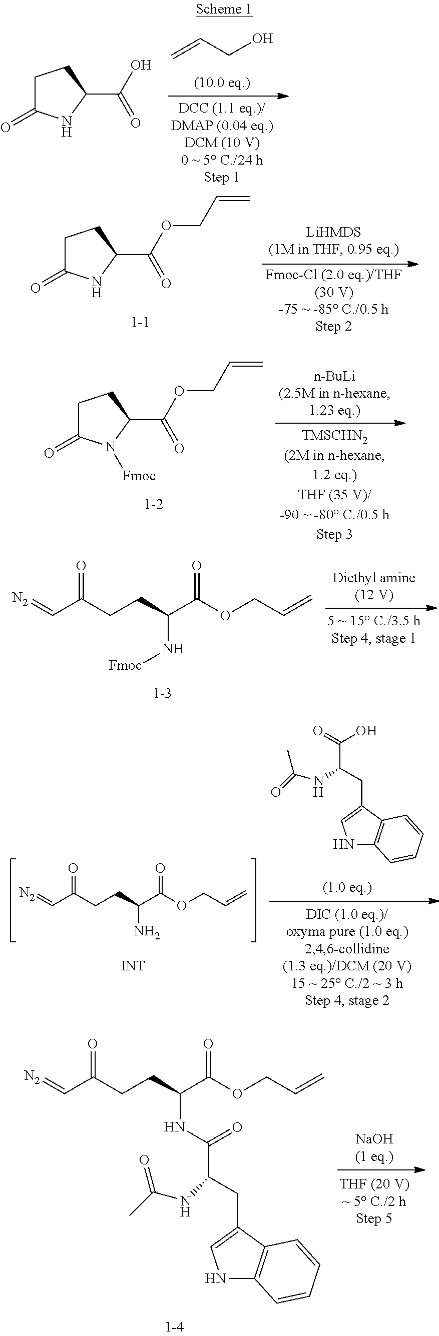

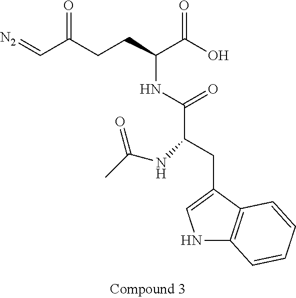

Step 1 Process Description:

1. Charged DCM (300 mL, 10 V) into a 500 mL reactor under N₂ protection.

2. Charged prop-2-en-1-ol (134.7 g, 10.0 eq.) into the above reactor with stirring.

3. Charged DCC (52.58 g, 1.1 eq.) into the reactor.

4. Charged DMAP (1.22 g, 0.04 eq.) into the reactor.

5. Cooled to 0° C.

6. Charged L-Pyroglutamic acid (30 g, 1.0 eq.) batchwise into the reactor below 5° C.

7. Stirred overnight (˜15 h) at 0˜5° C.

8. Sampled for IPC (3.4% SM left).

9. Charged another DCC (0.1 eq.) into the reactor.

10. Stirred for additional 7 h at 0˜5° C.

11. Sampled for IPC (SM:P=4.9%:72%).

12. Filtration and washed the solid with DCM (4 V).

13. Collected the filtrate and washed with water (3 V*1) and brine (3 V*1).

14. Concentrated the organic phase under vacuum until no solvent distilled out.

15. Charged MTBE (15 V) into the residue.

16. Stirred overnight at room temperature.

17. Filtered out the undissolved sticky semi-solid and washed with MTBE (2 V).

18. Combined the filtrate with another batch from 5 g of L-Pyroglutamic acid.

19. Concentrated the filtrate and switched with THF (4 V*2).

20. This resulted 53.9 g of crude product as a white solid (yield>100%) and used directly to the next step.

Step 2 Process Description

1. Charged THF (600 ml, 15 V) into a 1 L reactor under N₂ protection.

2. Charged compound 1-1 (40 g, 1.0 eq.) into the reactor with stirring.

3. Cooled to −80° C.

4. Charged dropwise LiHMDS (1 M in THF, 224 mL, 0.95 eq.) into the reactor mixture while maintaining the temperature at −80±5° C.

5. Stirred for −20 min (solution 1).

6. Charged THF (600 ml, 15 V) into another 5 L reactor under N₂ protection.

7. Charged Fmoc-Cl (122.1 g, 2.0 eq.) into the above 5 L reactor with stirring.

8. Cooled to −80° C.

9. Transferred the solution 1 obtained in step 5 into the above 5 L reactor slowly with stirring under N₂ press while maintaining the temperature at −80±5° C.

10. Stirred for 0.5 h at −80±5° C.

11. Sampled for IPC (no compound 1-1 left).

12. Quenched the reaction with sat. NH₄Cl (2.4 L, 60 V) until the pH was adjusted to 6˜7 while maintaining the temperature below −70° C.

13. Warmed to 0° C. and stopped stirring.

14. Phase separated and collected the above organic phase.

15. Washed the organic phase with half sat. brine (5 V*1) and sat. brine (5 V*1).

16. Combined the organic phase with another batch from 10 g of compound 1-1.

17. Concentrated the organic phase under vacuum.

18. Charged MTBE (8 V) and n-heptane (5 V) into the residue and stirred for 2 h at room temperature.

19. Filtration.

20. Re-slurry the solid with DCM (140 mL) for 30 min at room temperature.

21. Filtered out the solid and combined the filtrate in step 19.

22. Concentrated the filtrate under vacuum.

23. This residue ˜150 g crude sticky brown oil with poor purity and the residue was very difficult to purify.

24. Purified the crude product by column chromatography two times using DCM:PE (1:10˜1:0) as eluent

25. This resulted in ˜40 g sticky light yellow oil with ˜94% HPLC purity.

Step 3 Process Description

1. Charged THF (450 ml, 15 V) into a 1 L reactor under N₂ protection.

2. Charged TMSCHN₂ (2 M in n-hexane, 46.2 mL, 1.2 eq.) into the reactor with stirring.

3. Cooled to below −80° C.

4. Charged dropwise n-BuLi (2.5 M in n-hexane, 37.9 mL, 1.23 eq.) into the reactor while maintaining the temperature at −85±5° C.

5. Stirred for −30 min at −85±5° C. (solution 1).

6. Charged THF (600 mL, 20 V) into another 2 L reactor under N₂ protection.

7. Charged compound 1-2 (30 g, 1.0 eq.) into above 2 L reactor with stirring.

8. Cooled to below −90° C.

9. Transferred the solution 1 obtained in step 5 into the above 2 L reactor with stirring via N₂ press below −85° C.

10. Stirred for 15 min at −85±5° C.

11. Sampled for IPC (˜2.2% DRP104-M1-2 left).

12. Quenched the reaction with sat. NH₄C1 (300 mL, 10 V) while maintaining the temperature below −80° C.

13. Warmed to 0° C. and stopped stirring.

14. Phase separated and collected the above organic phase.

15. Washed the organic phase with half sat. brine (5 V*1) and sat. brine (5 V*1).

16. Combined the organic phase with another batch from 10 g of compound 1-2.

17. Concentrated the organic phase under vacuum and switched with MTBE (5 V*3) until the residue less than 3 V left.

18. Charged MTBE (5 V) into the residue and stirred 1-2 h at room temperature.

19. Filtration and washed the solid with MTBE (2 V).

20. Collected the solid and dried.

21. This resulting ˜30 g light yellow solid with ˜92.2% HPLC purity.

Step 4 Process Description

1. Charged Diethyl amine (100 mL, 12 V) into a 250 mL reactor under N₂ atmosphere.

2. Cooled to below 10° C.

3. Charged compound 1-3 (8.0 g, 1.0 eq.) into reactor with stirring.

4. Stirred for 3.5 hour at 10±5° C.

5. Sampled for HPLC analysis (no compound 1-3 left).

6. Concentrated the reaction mixture under vacuum at room temperature until the residue was no more than 2 V left.

7. Switched the residue with DCM (5 V) for three times until the residue was no more than 2 V left.

8. Diluted the residue with DCM (5 V) to give solution 1.

9. Charged DCM (15 V) into another reactor.

10. Charged N-acetyl-L-tryptophan (4.56 g, 1.0 eq.) into the above reactor with stirring.

11. Charged DIC (2.33 g, 1.0 eq.), oxyma pure (2.63 g, 1.0 eq.) and 2,4,6-collidine (2.92 g, 1.3 eq.) into the above reactor.

12. Cooled to below 10° C.

13. Charged solution 1 slowly into the above reactor.

14. Warmed to room temperature and stirred for 2-3 h at room temperature.

15. Sample for HPLC analysis (SM:P=8.0%:76.7%).

16. Combined with another batch (2 g).

17. Washed the above reaction mixture with 1M KHSO4 (3 V*1), water (3 V*2) and brine (3 V*1).

18. Concentrated the organic phase until the residue was no more than 2 V left.

19. Switched with EtOAc (5 V) for two times until the residue was no more than 2 V left.

20. Stirred overnight at room temperature.

21. Filtration and washed the solid with a little EtOAc (1 V).

22. Collected the solid and dried.

23. Obtained 9.0 g light yellow solid with 97.8% HPLC purity.

Step 5 Process Description

1. Charged THF (80 mL, 20 V) into a 250 mL reactor.

2. Charged compound 1-4 (4.0 g, 1.0 eq.) into reactor with stirring.

3. Cooled to below 10° C.

4. Charged NaOH (1 M, 9.1 mL, 1.0 eq.) into reactor while maintaining the temperature below 10° C.

5. Sampled for HPLC analysis after reacting for 2 h at 0˜10° C. (no compound 1-4 left)

6. Charged AcOH (1.0 eq.) into the reaction mixture at 0˜10° C.

7. Charged water (10 V) into the above reaction mixture after stirring for 20 min.

8. The organic solvent was distilled out under vacuum.

9. ˜6 g crude semi-solid obtained after freeze-drying.

10. Compound 3 (˜600 mg; brown solid) was obtained with 80.2% HPLC purity after further purification by SFC (using MeOH as eluent). ¹H and ¹³C NMR was consistent with the structure.

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications and publications cited herein are fully incorporated by reference herein in their entirety.

1. A method of treating a subject having cancer, the method comprising administering to the subject in need thereof a therapeutically effective amount of:
(a) isopropyl (S)-2-((S)-2-acetamido-3-(1H-indol-3-yl)propanamido)-6-diazo-5-oxohexanoate, or a pharmaceutically acceptable salt or solvate thereof; or
(b) isopropyl (S)-2-((S)-6-acetamido-2-((3S,5S,7S)-adamantane-1-carboxamido) hexanamido)-6-diazo-5-oxohexanoate, or a pharmaceutically acceptable salt thereof;
(c) (S)-2-((S)-2-acetamido-3-(1H-indol-3-yl)propanamido)-6-diazo-5-oxohexanoic acid, or a pharmaceutically acceptable salt thereof; or
(d) 6-diazo-5-oxo-L-norleucine; and
(e) an immune checkpoint inhibitor,
wherein the isopropyl (S)-2-((S)-2-acetamido-3-(1H-indol-3-yl)propanamido)-6-diazo-5-oxohexanoate, or a pharmaceutically acceptable salt or solvate thereof, or
isopropyl (S)-2-((S)-6-acetamido-2-((3S,5S,7S)-adamantane-1-carboxamido) hexanamido)-6-diazo-5-oxohexanoate, or a pharmaceutically acceptable salt thereof, or
(S)-2-((S)-2-acetamido-3-(1H-indol-3-yl)propanamido)-6-diazo-5-oxohexanoic acid, or a pharmaceutically acceptable salt thereof, or
6-diazo-5-oxo-L-norleucine, or a pharmaceutically acceptable salt thereof, is administered to the subject according to an intermittent dosing schedule,
and wherein the immune checkpoint inhibitor is selected from the group consisting of an anti-PD-1 antibody, an anti-PD-L1 antibody, an anti-CTLA-4 antibody, an anti-LAG3 antibody, and an anti-TIM3 antibody.

2-4. (canceled)

5. The method of claim 1, wherein the anti-PD-1 antibody is selected from the group consisting of nivolumab, pembrolizumab, pidilizumab, STI-A1110, PDR001, MEDI 0680, AGEN2034, BGB-A317, AB122, TSR-042, PF-06801591, cemiplimab, SYM021, JNJ 63723283, HLX10, LZM009, and MGA012.

6. (canceled)

7. (canceled)

8. The method of claim 1, wherein the anti-PD-L1 antibody is selected from the group consisting of avelumab, atezolizumab, durvalumab, and STI-A1014.

9. (canceled)

10. (canceled)

11. The method of claim 1, wherein the anti-CTLA-4 antibody is selected from the group consisting of ipilimumab and tremelimumab.

12. (canceled)

13. (canceled)

14. The method of claim 1, wherein the anti-LAG3 antibody is GSK2831781.

15. (canceled)

16. The method of claim 15, wherein the TIM3 inhibitor is an anti-TIM3 antibody.

17. The method of claim 1, wherein the cancer is or has become resistant to treatment with at least one immune checkpoint inhibitor.

18-21. (canceled)

22. The method of claim 1, wherein the cancer is a solid tumor.

23. The method of claim 1, wherein the cancer is a hematological cancer.

24. (canceled)

25. The method of claim 1, wherein the cancer is selected from the group consisting of hepatocellular carcinoma, glioblastoma, lung cancer, breast cancer, head and neck cancer, prostate cancer, melanoma, and colorectal cancer.

26. (canceled)

27. The method of claim 1, wherein isopropyl (S)-2-((S)-2-acetamido-3-(1H-indol-3-yl)propanamido)-6-diazo-5-oxohexanoate, or a pharmaceutically acceptable salt or solvate thereof, or isopropyl (S)-2-((S)-6-acetamido-2-((3S,5S,7S)-adamantane-1-carboxamido)hexanamido)-6-diazo-5-oxohexanoate, or a pharmaceutically acceptable salt thereof, or (S)-2-((S)-2-acetamido-3-(1H-indol-3-yl)propanamido)-6-diazo-5-oxohexanoic acid, or a pharmaceutically acceptable salt thereof, or 6-diazo-5-oxo-L-norleucine is administered to the subject three times a week on non-consecutive days.

28-32. (canceled)

33. A method of treating a subject having cancer, the method comprising administering to the subject in need thereof a therapeutically effective amount of isopropyl (S)-2-((S)-2-acetamido-3-(1H-indol-3-yl)propanamido)-6-diazo-5-oxohexanoate, or a pharmaceutically acceptable salt or solvate thereof, or isopropyl (S)-2-((S)-6-acetamido-2-((3S,5S,7S)-adamantane-1-carboxamido)hexanamido)-6-diazo-5-oxohexanoate, or a pharmaceutically acceptable salt thereof, or (S)-2-((S)-2-acetamido-3-(1H-indol-3-yl)propanamido)-6-diazo-5-oxohexanoic acid, or a pharmaceutically acceptable salt thereof, according to an intermittent dosing schedule.

34. The method of claim 33, wherein the cancer is a solid tumor.

35. The method of claim 33, wherein the cancer is a hematological cancer.

36. (canceled)

37. The method of claim 33, wherein the cancer is selected from the group consisting of hepatocellular carcinoma, glioblastoma, lung cancer, breast cancer, head and neck cancer, prostate cancer, melanoma, and colorectal cancer.

38. (canceled)

39. The method of claim 33, wherein isopropyl (S)-2-((S)-2-acetamido-3-(1H-indol-3-yl)propanamido)-6-diazo-5-oxohexanoate, or a pharmaceutically acceptable salt or solvate thereof, or isopropyl (S)-2-((S)-6-acetamido-2-((3S,5S,7S)-adamantane-1-carboxamido)hexanamido)-6-diazo-5-oxohexanoate, or a pharmaceutically acceptable salt thereof, or (S)-2-((S)-2-acetamido-3-(1H-indol-3-yl)propanamido)-6-diazo-5-oxohexanoic acid, or a pharmaceutically acceptable salt thereof, is administered to the subject three times a week on non-consecutive days.

40-43. (canceled)

44. The method of claim 1, wherein isopropyl (S)-2-((S)-2-acetamido-3-(1H-indol-3-yl)propanamido)-6-diazo-5-oxohexanoate, or a pharmaceutically acceptable salt or solvate thereof, is administered to the subject.

45. (canceled)

46. The method of claim 1, wherein (S)-2-((S)-2-acetamido-3-(1H-indol-3-yl)propanamido)-6-diazo-5-oxohexanoic acid, or a pharmaceutically acceptable salt thereof, is administered to the subject.

47. A method of treating a subject having cancer, the method comprising administering to the subject in need thereof a therapeutically effective amount of 6-diazo-5-oxo-L-norleucine for 5 consecutive days in a row followed by 2 consecutive days in a row wherein 6 diazo-5-oxo-L-norleucine is not administered.

48-55. (canceled)

56. (S)-2-((S)-2-acetamido-3-(1H-indol-3-yl)propanamido)-6-diazo-5-oxohexanoic acid, or a pharmaceutically acceptable salt thereof.

57. A pharmaceutical composition comprising the compound of claim 56, or a pharmaceutically acceptable salt thereof, and a pharmaceutically acceptable excipient.
